(19) United States
(12) Patent Application Publication
(10) Pub. No.: US 2017/0175148 A1
(43) Pub. Date: Jun. 22, 2017

(54) Recombinant Cyanobacterial Cell For Contamination Control In A Cyanobacterial Culture Producing A Chemical Compound Of Interest

(71) Applicant: Algenol Biotech LLC, Fort Myers, FL (US)

(72) Inventors: Dan Kramer, Berlin (DE); Christian Weissert, Berlin (DE); John Coleman, Toronto (CA); Heike Enke, Berlin (DE); Eva Schunder, Berlin (DE); Charles Ryan Budinoff, Fort Myers, FL (US); William Patrick Porubsky, Fort Myers, FL (US); Laura Belicka, Fort Myers, FL (US)

(73) Assignee: Algenol Biotech LLC, Fort Myers, FL (US)

(21) Appl. No.: 15/389,003

(22) Filed: Dec. 22, 2016

Related U.S. Application Data
(60) Provisional application No. 62/271,197, filed on Dec. 22, 2015.

(51) Int. Cl. C12P 7/06; C12N 15/74

(52) U.S. Cl. CPC C12P 7/065; C12Y 401/01001; C12N 15/74

(57) ABSTRACT

A non-naturally occurring cyanobacterial cell for the production of a chemical compound of interest is provided which contains at least one genetic modification that alters expression of at least one phosphate uptake regulating gene encoding a protein involved in regulation of phosphate metabolism, so that cellular uptake and/or intracellular storage of a phosphate compound is increased in comparison to a native form of the cyanobacterial cell. Related methods and uses involving the cyanobacterial cell are also provided.

### CROSS-REFERENCE TO RELATED APPLICATIONS

This application claims the benefit of U.S. Provisional Application No. 62/271,197, filed on Dec. 22, 2015, the disclosure of which is hereby incorporated by reference.

### STATEMENT REGARDING FEDERALLY SPONSORED RESEARCH OR DEVELOPMENT

Not applicable.

### REFERENCE TO SEQUENCE LISTING

This application contains a sequence listing submitted by EFS-Web, thereby satisfying the requirements of 37 C.F.R. §§1.821-1.825. The sequence listing, entitled “P0041_02_US_ST25”, contains 47 sequences. The sequence listing is 136 KB in size, and was created on Dec. 22, 2016.

### FIELD OF INVENTION

The present invention relates to recombinant cyanobacterial cells for the production of a chemical compound of interest. In particular, the present invention relates to genetic modifications that can improve competitiveness of recombinant cyanobacterial cells over heterotrophic contaminants in a cyanobacterial culture.

### BACKGROUND

Cyanobacteria are prokaryotes capable of photoautotrophy. It is known that cyanobacteria can be genetically modified to use light and CO₂ to produce compounds of interest such as biofuels, industrial chemicals, pharmaceuticals, nutrients, carotenoids, and food supplements. One of these compounds is ethanol.

Various cyanobacterial strains have been genetically enhanced to produce compounds of interest. For example, transformation of the cyanobacterial genus Synechocystis has been described in WO 2009/098089 A2 and WO 2011/018116 A1. Transformation of Cyanobacterium sp. is described in WO 2014/100799 A2. These publications are hereby used to describe more fully the state of the art to which this invention pertains.

A remaining challenge in cyanobacterial production of compounds of interest is to achieve commercially viable production targets. Therefore, there is a need for improved cyanobacterial cells which increase the economic efficiency of this technology.

### SUMMARY

This invention provides a recombinant, i.e. non-naturally occurring, cyanobacterial cell for the production of a chemical compound of interest. The cyanobacterial cell can contain, for example, at least one genetic modification that alters the expression of a phosphate uptake regulating gene encoding a protein involved in regulation of phosphate metabolism, so that cellular uptake and/or intracellular storage of a phosphate compound is increased in comparison to a native form of the cyanobacterial cell, in particular when grown under the same conditions. The cyanobacterial cell can additionally contain at least one recombinant production gene encoding an enzyme for the production of the chemical compound of interest.

This invention further provides a method for producing a chemical compound of interest with the above-mentioned recombinant cyanobacterial cell. The method comprises culturing the cyanobacterial cell in a phosphate compound-poor medium containing from ≧0 to 100 μM of the phosphate compound. During culturing, the cyanobacterial cell can express the enzyme, thereby producing the chemical compound of interest.

This invention also provides use of the above-mentioned recombinant cyanobacterial cell in a cyanobacterial culture for reducing or preventing loss of the produced chemical compound of interest caused by heterotrophic microorganisms.

Finally, this invention also provides a method for producing the above-mentioned cyanobacterial cell. The method comprises providing at least one transformable nucleic acid construct for the genetic modification that alters the expression of at least one phosphate uptake regulating gene encoding a protein involved in regulation of phosphate metabolism and at least one transformable nucleic acid construct comprising the at least one recombinant production gene encoding an enzyme for the production of the chemical compound of interest. The transformable nucleic acid constructs can be transformed into a cyanobacterial cell to obtain the recombinant cyanobacterial cell of the present invention.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE SEVERAL VIEWS OF THE DRAWINGS

Embodiments of the invention will be described below with reference to the following figures.

FIG. 1 depicts a vector map (FIG. 1A) with annotations (FIG. 1B) and nucleotide sequence (FIG. 1C, SEQ ID NO: 1) of synthetically produced plasmid construct #1980 TK665\oriVT-orf0666-up_FRT-Porf0221-galK(opt)-PcpcB171-Gm**-ter-FRT_orf0666-down. The plasmid contains a deletion cassette for knockout of the negative phosphate uptake regulator orf0666 in Cyanobacterium sp. ABICyano1 deposited in the American Type Culture Collection (ATCC) under ATCC accession number PTA-13311 by homologous double-crossover integration via orf0666-up and orf0666-down flanking sequences. FRT sites flanking a codon-adapted gentamycin resistance marker driven by the PcpcB promoter are included in the DNA between the orf0666-up and orf0666-down flanking sequences.

FIG. 2 depicts a vector map (FIG. 2A) with annotations (FIG. 2B, SEQ ID NO: 2, SEQ ID NO: 3, and SEQ ID NO: 4) and nucleotide sequence (FIG. 2C, SEQ ID NO: 5) of synthetically produced plasmid construct #1904 pAB1_6.8::PnirA*2-PDC(AB1opt1)-TdsrA-PcpcB-ADH111(AB1opt)-TrbcS. The plasmid contains a variant of the PnirA promoter upstream of a codon improved pdc gene from Zymomonas mobilis and the PcpcB promoter upstream of a codon improved adh gene from Lyngbya sp., termed ADH111.

FIG. 3 depicts a vector map (FIG. 3A) with annotations (FIG. 3B, SEQ ID NO: 6, SEQ ID NO: 7, and SEQ ID NO: 8) and nucleotide sequence (FIG. 3C, SEQ ID NO: 9) of synthetically produced plasmid construct #2042 pAB722\pAB1-6.8::PnirA*2-PDC(AB1opt1)-dsrA-Prbc*(optRBS)-synADH-Toop. The plasmid contains a variant of the PnirA promoter upstream of a codon improved pdc gene from Zymomonas mobilis and a variant of the Prbc promoter with optimised ribosome binding site upstream of a codon improved adh gene from Synechocystis PCC6803, termed SynADH.

FIG. 4 shows a graphical evaluation of the phosphate uptake rate (FIG. 4A) and the cellular phosphate storage level (FIG. 4B, FIG. 4C) of ethanologenic Cyanobacterium sp. PTA 13311 strain AB0670 according to the present invention in comparison to ethanologenic Cyanobacterium sp. PTA 13311 reference strain AB0250, termed “Control” under different conditions.

FIG. 5 shows a graphical evaluation of the time-dependent liquid ethanol content (FIG. 5A) and Alcanivorax content (FIG. 5B) in cultures of strain AB0670 and reference strain AB0250.

FIG. 6 shows a graphical evaluation of the time-dependent development of optical density (FIG. 6A) and ethanol accumulation (FIG. 6B) in semi-continuously operated outdoor photobioreactors with cultures of ethanologenic, phoU (orf 0666) knock-out, Cyanobacterium sp. PTA 13311 strain AB0042 according to the present invention and ethanologenic Cyanobacterium sp. PTA 13311 reference strain AB0382.

FIG. 7 shows amino acid sequences of PstS encoded by AB1_orf3306 (FIG. 7A, SEQ ID NO: 10), PstS2 encoded by AB1_orf1463 (FIG. 7B, SEQ ID NO: 11) and PstS3 encoded by AB1_orf0749 (FIG. 7C, SEQ ID NO: 12), as well as the amino acid sequence of the PhoU homolog encoded by AB1_orf0666 (FIG. 7D, SEQ ID NO: 13).

FIG. 8 shows nucleotide sequences of promoters PrpsL (FIG. 8A, SEQ ID NO: 14), PcpcB (FIG. 8B, SEQ ID NO: 15), PrbcL (FIG. 8C, SEQ ID NO: 16), PpsaA (FIG. 8D, SEQ ID NO: 17), PpsbB (FIG. 8E, SEQ ID NO: 18) and PatpG (FIG. 8F, SEQ ID NO: 19) which are constitutive or at least facultatively constitutive under typical cyanobacterial culturing conditions. FIG. 8G (SEQ ID NO: 20) shows the nucleotide sequence of the promoter PpetE which responds to copper levels.

FIG. 9 shows nucleotide sequences of metal inducible promoters PziaA (FIG. 9A, SEQ ID NO: 21), PaztA (FIG. 9B, SEQ ID NO: 22), PsmtA (FIG. 9C, SEQ ID NO: 23), PcorT (FIG. 9D, SEQ ID NO: 24), PnrsB (FIG. 9E, SEQ ID NO: 25), Porf0316 (FIG. 9F, SEQ ID NO: 26), Porf0221 (FIG. 9G, SEQ ID NO: 27), Porf0223 (FIG. 9H, SEQ ID NO: 28), Porf3126 (FIG. 9I, SEQ ID NO: 29), PmntC (FIG. 9J, SEQ ID NO: 30) and PpetJ (FIG. 9K, SEQ ID NO: 31).

FIG. 10 shows nucleotide sequences of nitrate inducible promoters PnirA (FIG. 10A, (SEQ ID NO: 32), PnrtA (FIG. 10B, (SEQ ID NO: 33) and PnarB (FIG. 10C, (SEQ ID NO: 34).

FIG. 11 shows amino acid sequences of Pdc enzymes from Zymomonas mobilis (FIG. 11A, SEQ ID NO: 35) and Zymobacter palmae (FIG. 11B, SEQ ID NO: 36).

FIG. 12 shows amino acid sequences of Adh enzymes from Lyngbya sp. (FIG. 12A, SEQ ID NO: 37), Microcystis aeruginosa (FIG. 12B, SEQ ID NO: 38), Synechococcus sp. (FIG. 12C, SEQ ID NO: 39) and Synechocystis PCC6803 (FIG. 12D, SEQ ID NO: 40).

FIG. 13 depicts a vector map (cc250) with annotations for a synthetic plasmid configured with a deletion cassette for knockout of the negative phosphate uptake regulator PhoU in Synechococcus PCC 7002.

FIG. 14 depicts a vector map (cc252) with annotations for a synthetic plasmid configured with a deletion cassette for knockout of the negative phosphate uptake regulator PhoU in Anabaena variabilis.

FIG. 15A is a line graph showing the competition dynamics of contaminant bacterial cell counts over time in ethanologenic cyanobacterial cultures having the phoU knockout, pre-loaded with three different levels of phosphoric acid (150 μM/OD₇₅₀, 300 μM/OD₇₅₀, or 450 μM/OD₇₅₀, as indicated. Contamination of the cultures with heterotrophic bacteria (measured as cfu/ml) was determined over time. The cultures with a higher pre-loading with phosphate (prior to inoculation with the contaminants) were able to maintain a lower level of contamination throughout the 17 day run.

FIG. 15B is a line graph showing ethanol production over time, in ethanologenic cyanobacterial cultures with the phoU knockout, pre-loaded with three different levels of phosphoric acid (150 μM/OD₇₅₀, 300 μM/OD₇₅₀, or 450 μM/OD₇₅₀, as indicated.

FIG. 16A is a line graph showing the hourly uptake of a high level of phosphoric acid pre-load, measured as the amount of phosphorus remaining in the culture supernatant over a 68 hour period. The cumulative amount of phosphorus that was fed to the cultures is also shown (dashed line). This graph indicates that a large amount of phosphorus could be taken up by the recombinant cells, particularly when the phosphorus is in the form of phosphoric acid.

FIG. 16B is a line graph showing the intracellular phosphorus (P) concentration of an ethanologenic, phoU knock-out cyanobacterial culture with either no P loading (black) or with a high level of P loading (grey). The graph shows that the cells that were highly phosphorus pre-loaded could take up a large amount of phosphorus, and thus did not need the addition of phosphorus at day 4, 8, 12, 17, and 22.

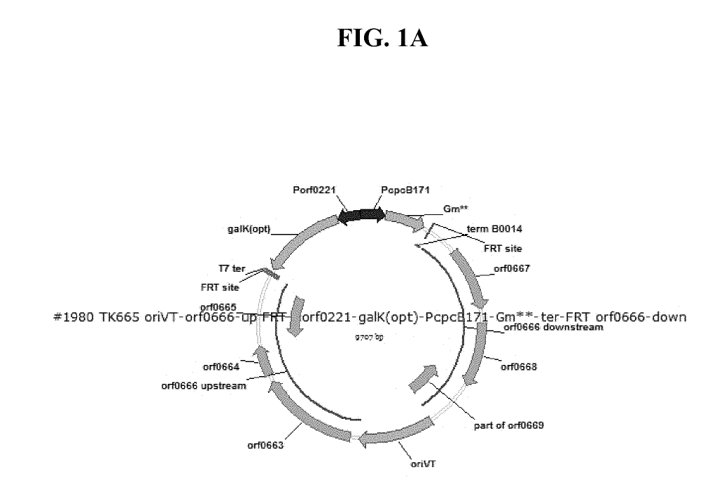

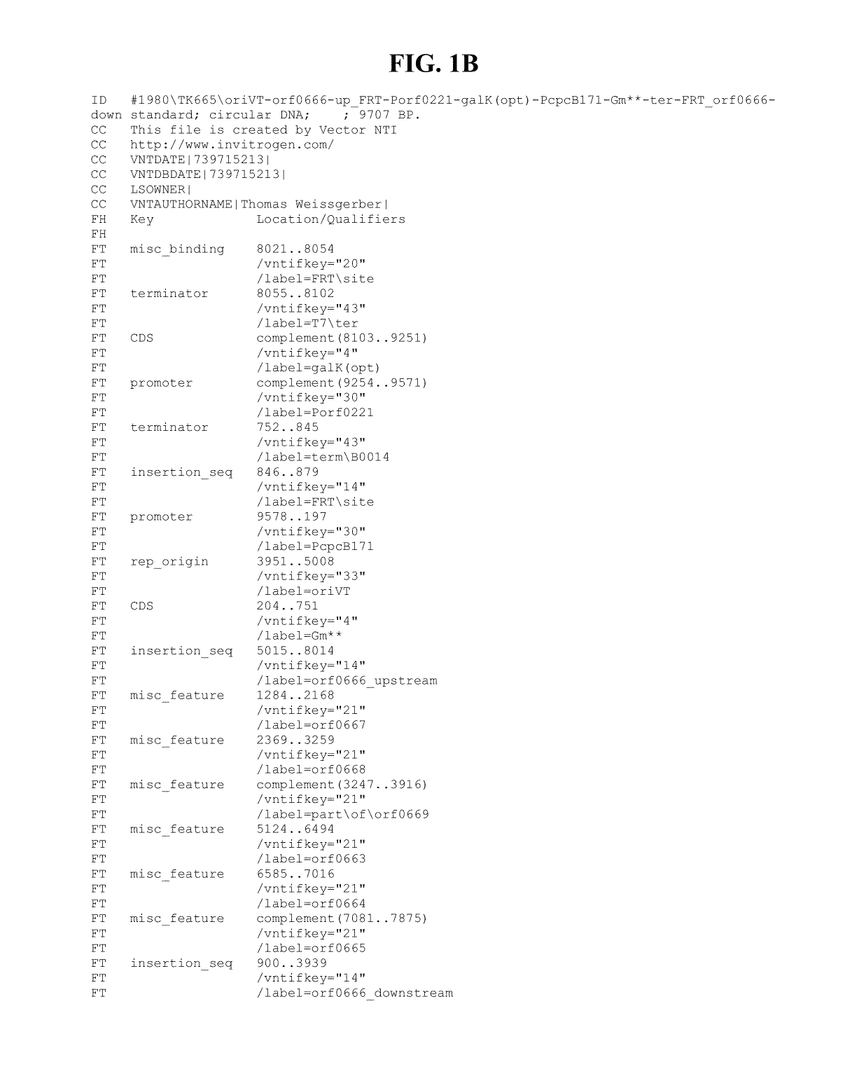

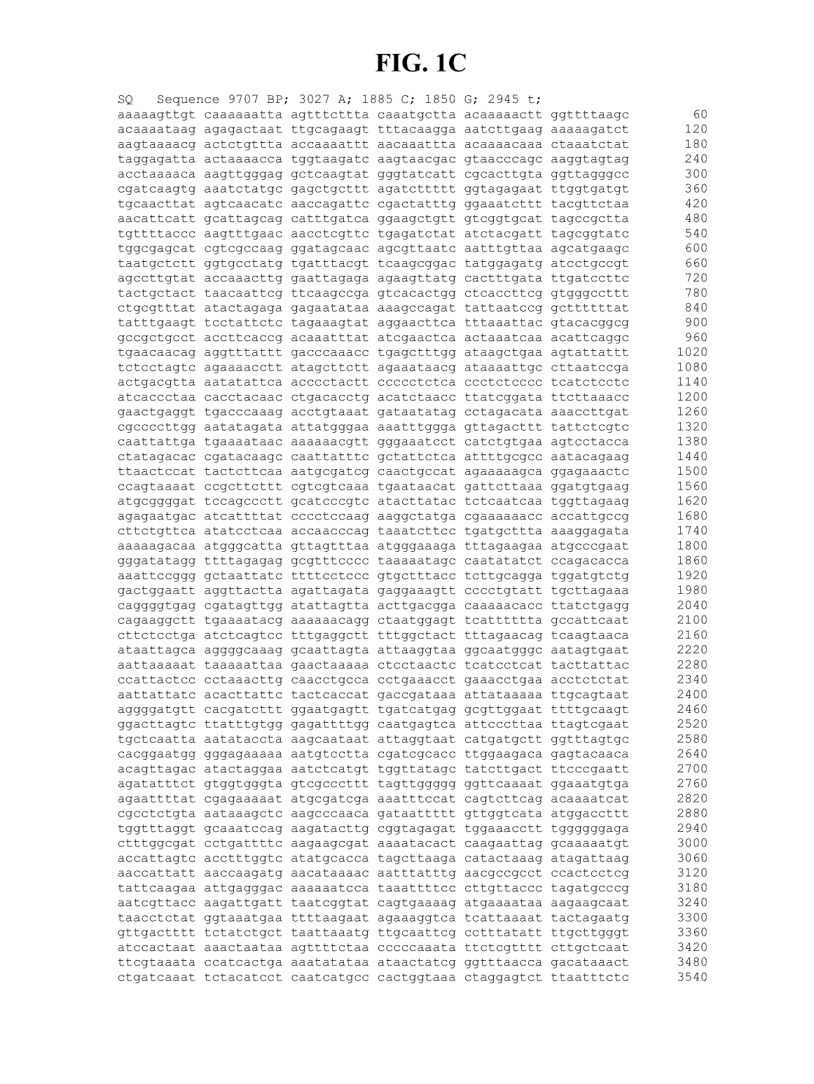

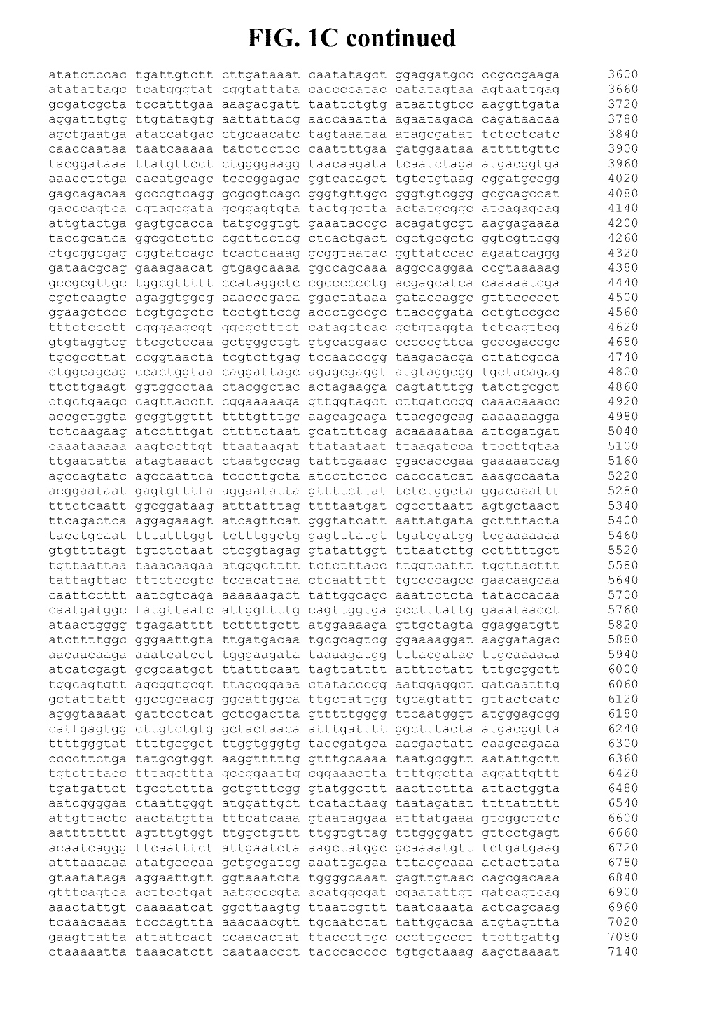

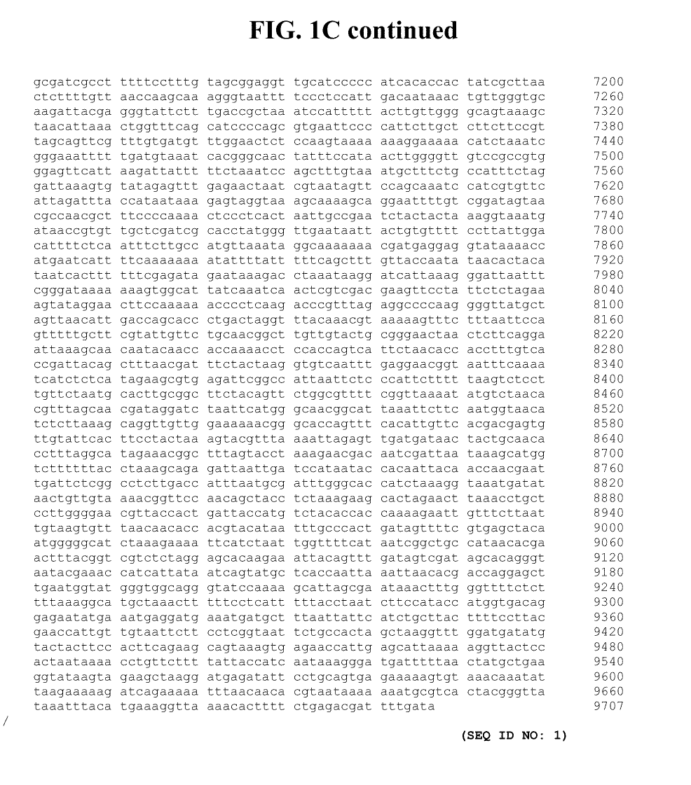

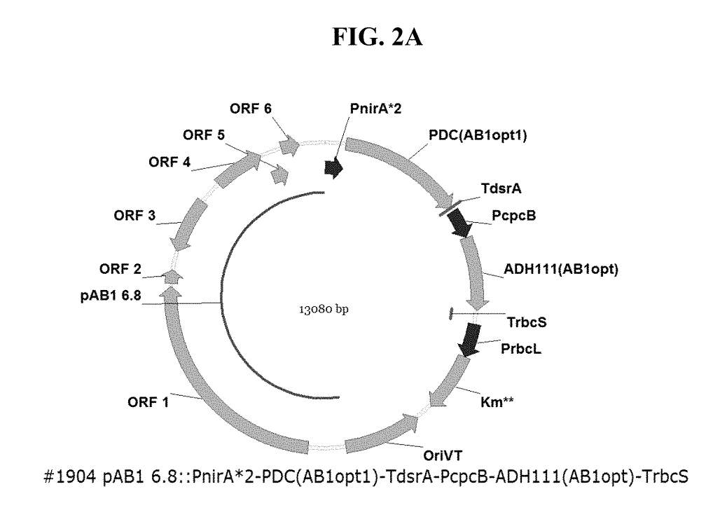

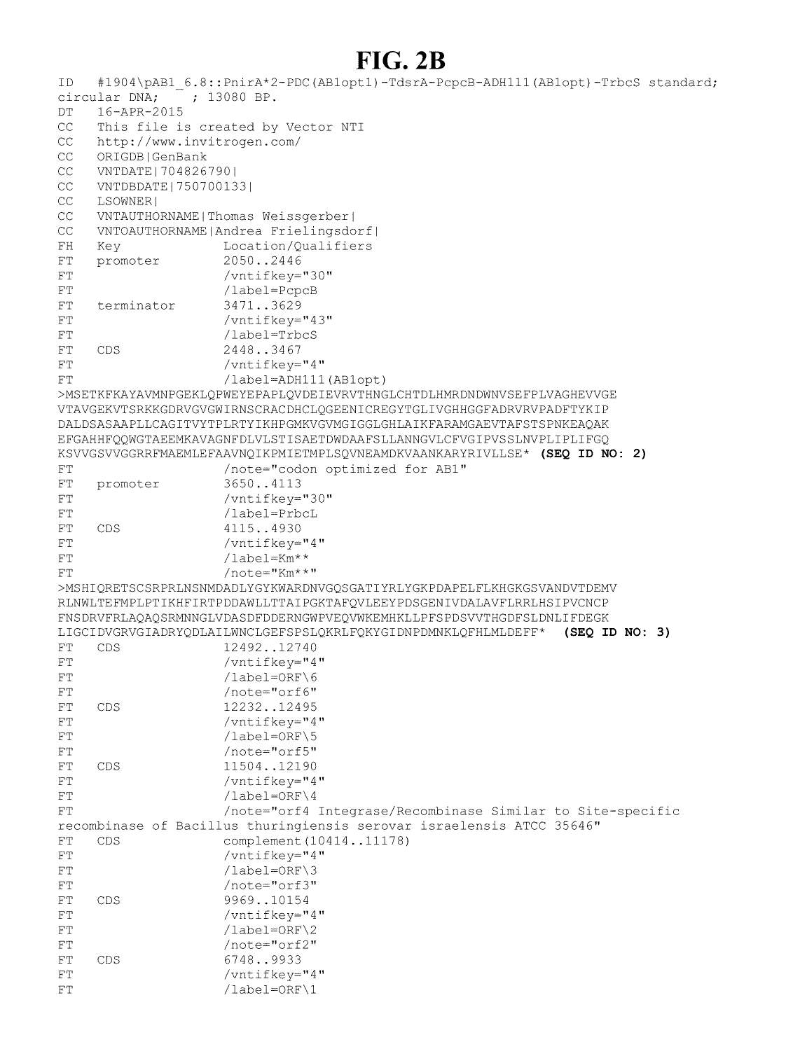

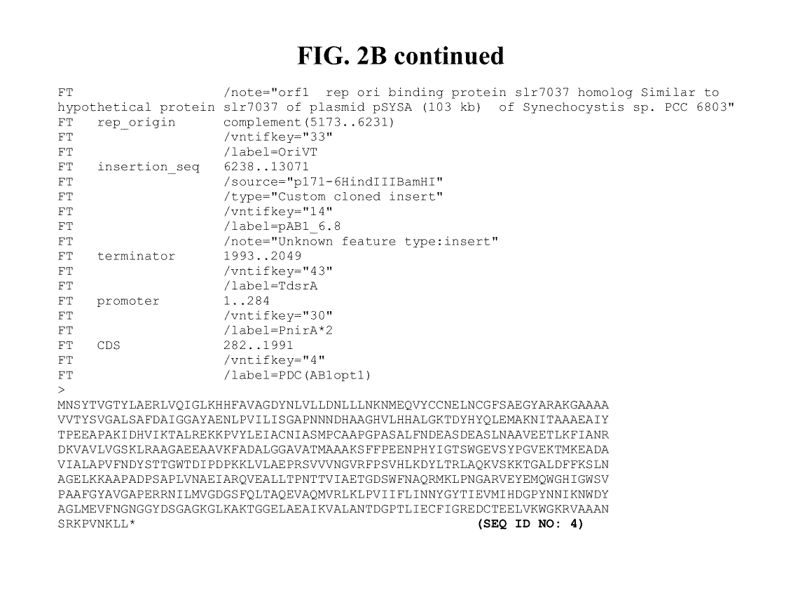

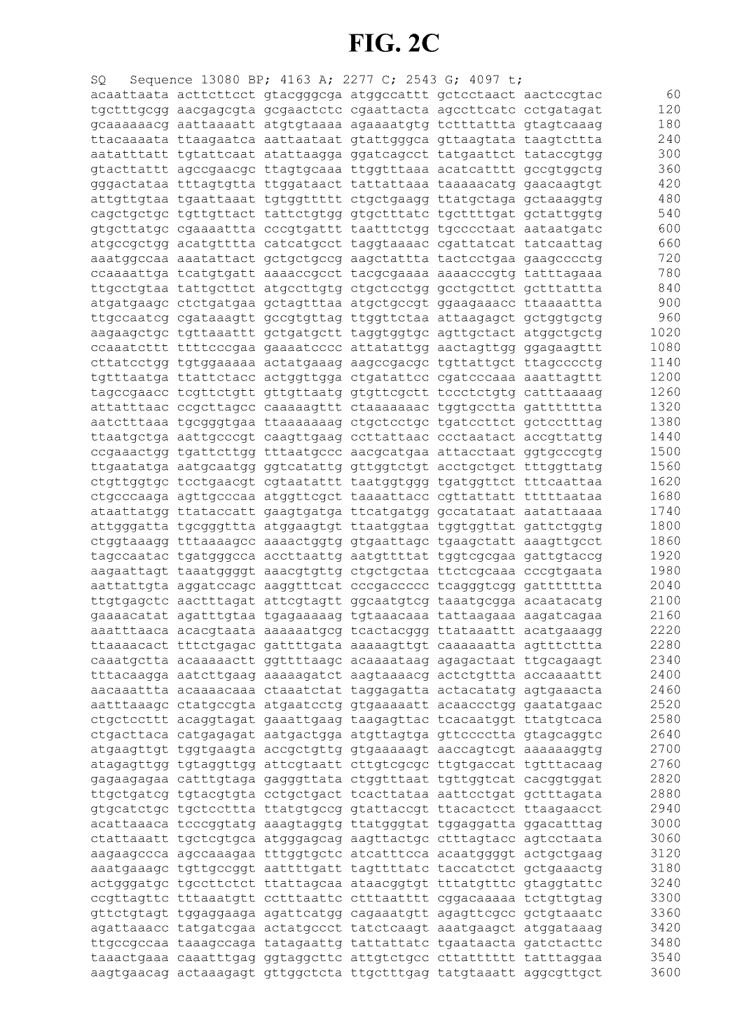

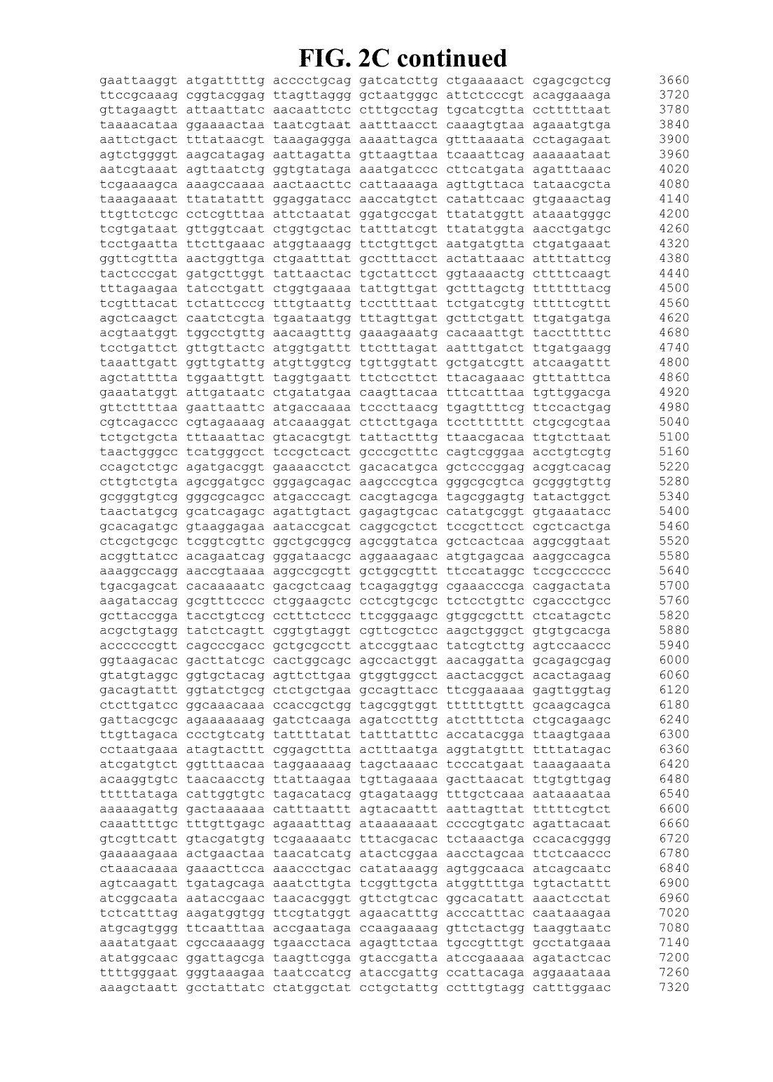

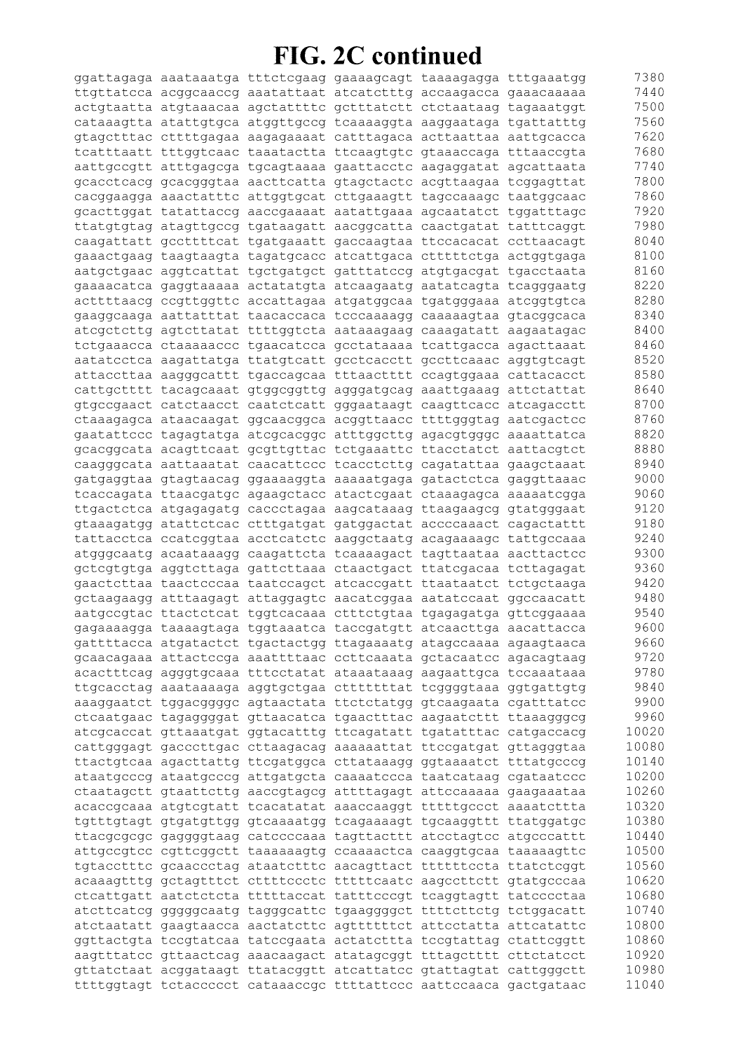

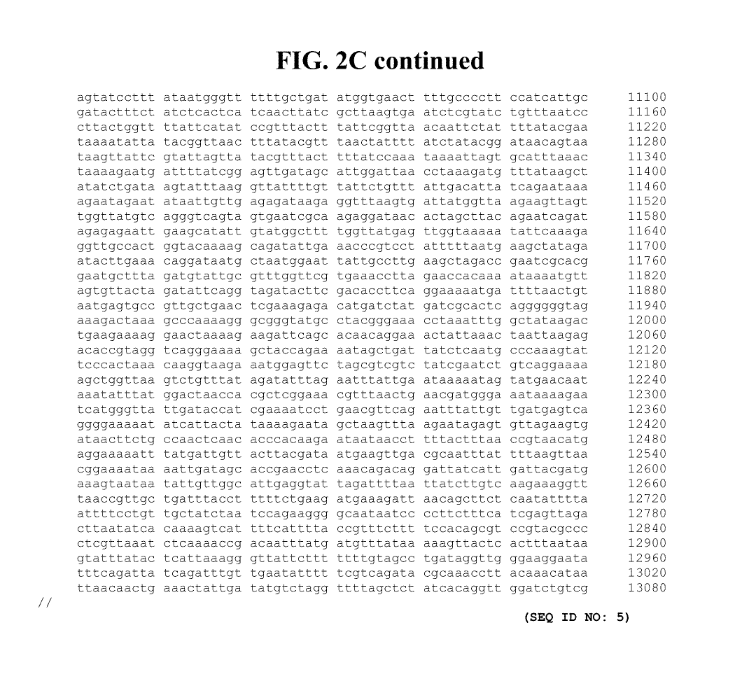

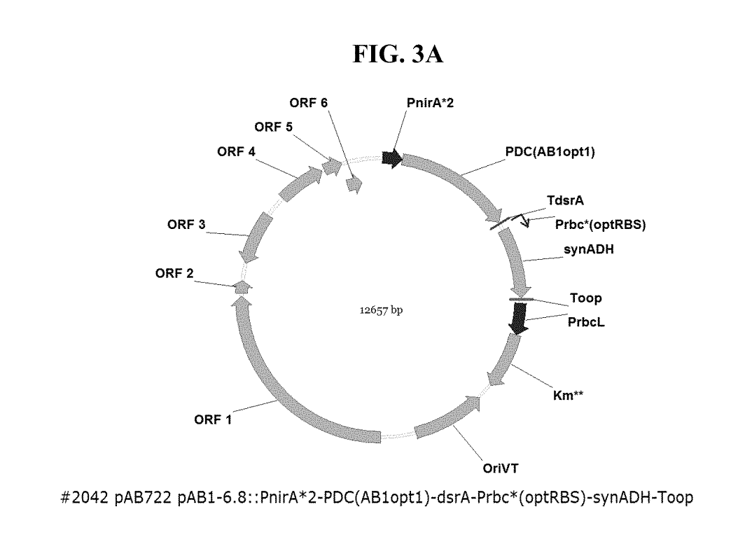

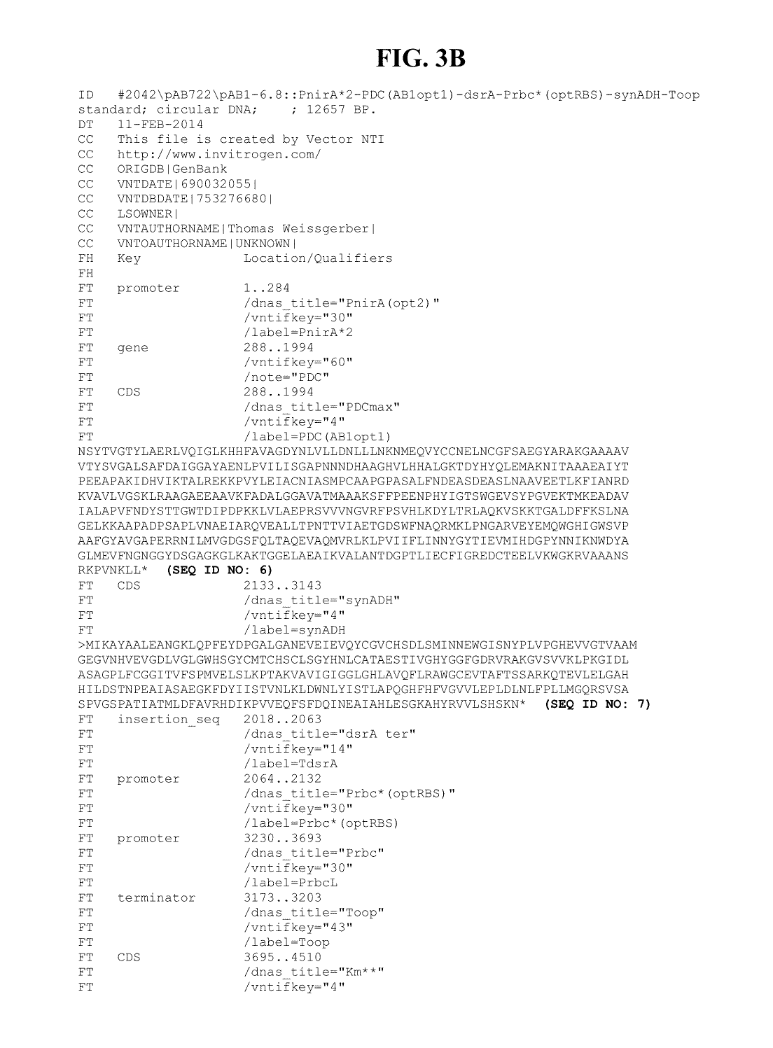

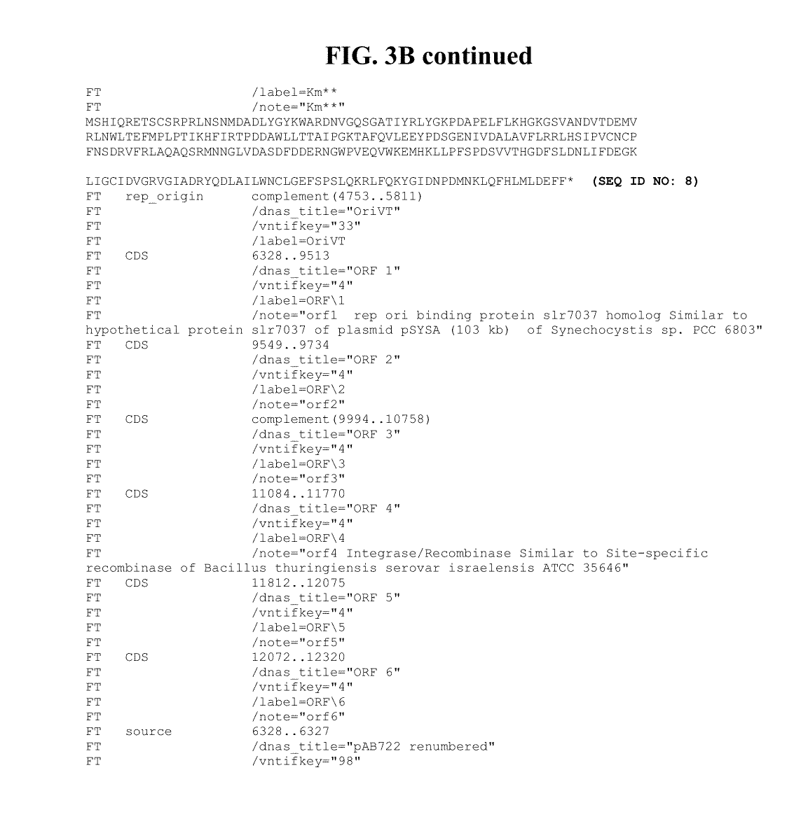

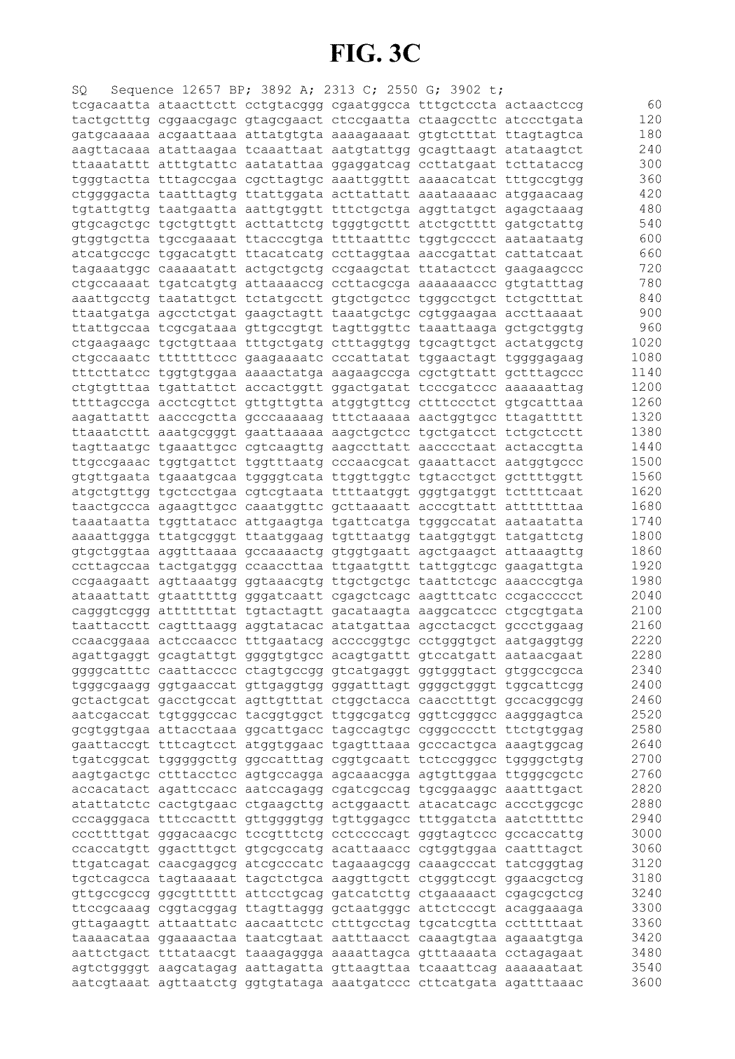

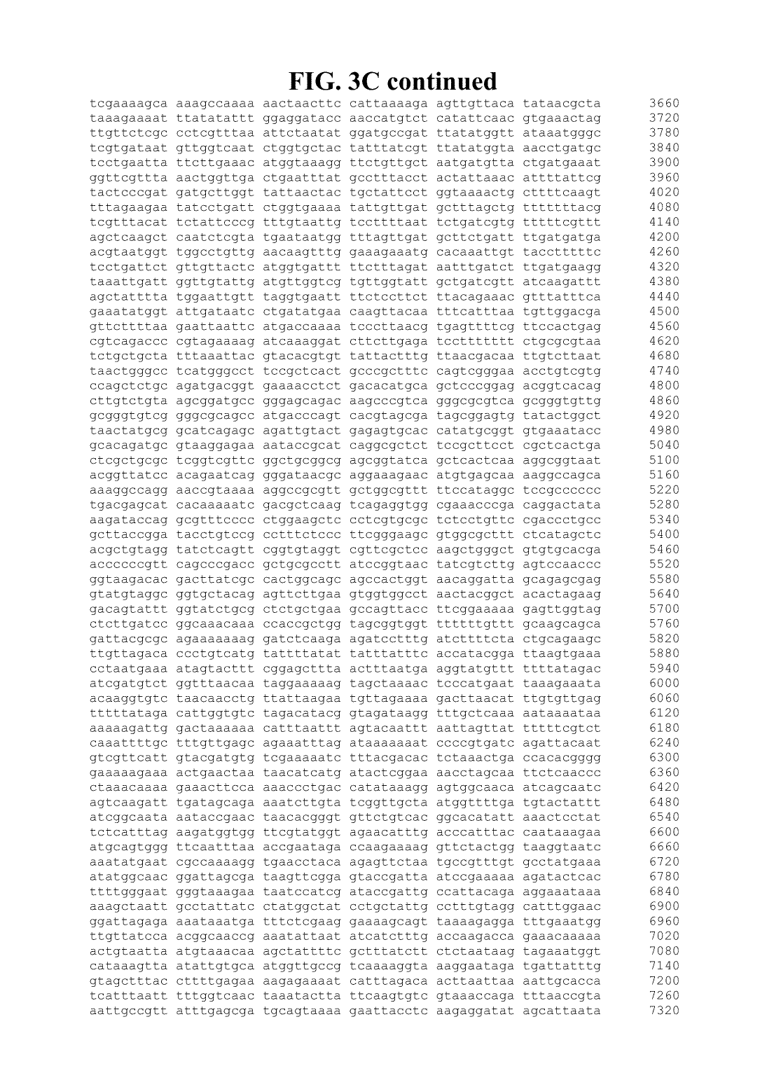

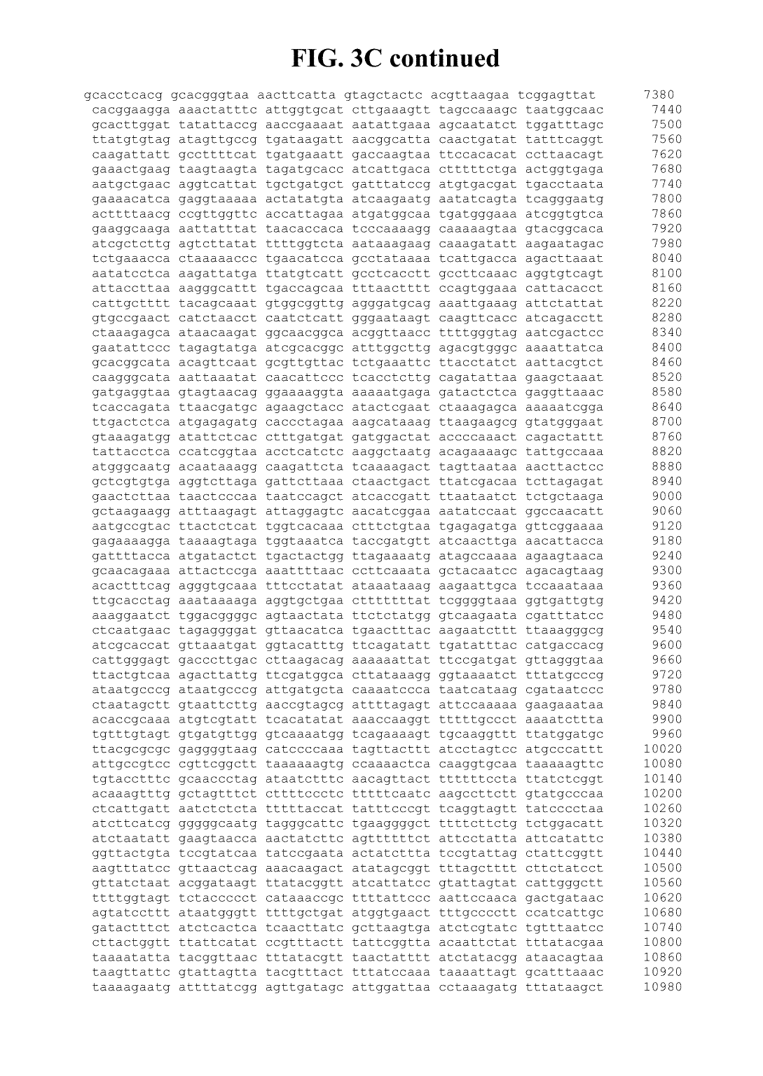

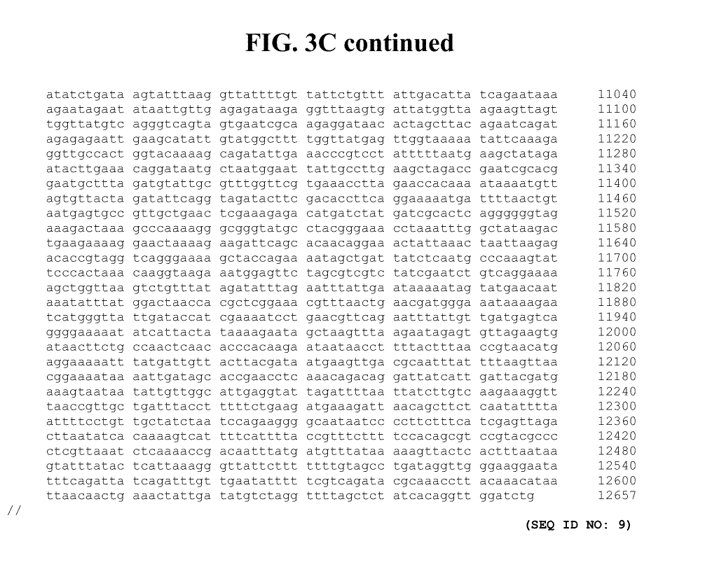

### DETAILED DESCRIPTION

In a first aspect, this invention provides a recombinant cyanobacterial cell for the production of a chemical compound of interest, containing
a) at least one genetic modification that alters an expression of at least one phosphate uptake regulating gene encoding a protein involved in regulation of phosphate metabolism, wherein cellular uptake and/or intracellular storage of a phosphate compound is increased in comparison to a native form of the cyanobacterial cell, in particular when grown under the same conditions, and
b) at least one recombinant production gene encoding an enzyme for the production of the chemical compound of interest.

### DEFINITIONS

Unless defined otherwise, all technical and scientific terms used herein have the meaning commonly understood by a person skilled in the art to which this invention belongs. As used herein, the following terms have the meanings ascribed to them unless specified otherwise.

The term “about” is used herein to mean approximately, in the region of, roughly, or around. When the term “about” is used in conjunction with a numerical value/range, it modifies that value/range by extending the boundaries above and below the numerical value(s) set forth. In general, the term “about” is used herein to modify a numerical value(s) above and below the stated value(s) by a variance of 20%.

The term “Cyanobacterium” refers to a member from the group of photoautotrophic prokaryotic microorganisms which can utilize solar energy and fix carbon dioxide. Cyanobacteria are also referred to as blue-green algae.

The terms “host cell” and “recombinant host cell” are intended to include a cell suitable for metabolic manipulation, e.g., which can incorporate heterologous polynucleotide sequences, e.g., which can be transformed. The term is intended to include progeny of the cell originally transformed. In particular embodiments, the cell is a prokaryotic cell, e.g., a cyanobacterial cell. The term recombinant host cell is intended to include a cell that has already been selected or engineered to have certain desirable properties and to be suitable for further genetic enhancement.

“Competent to express” refers to a host cell that provides a sufficient cellular environment for expression of endogenous and/or exogenous polynucleotides.

The terms “polynucleotide” and “nucleic acid” also refer to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs. It will be understood that, where required by context, when a nucleotide sequence is represented by a DNA sequence (i.e., A, T, G, C), this also includes an RNA sequence (i.e., A, U, G, C) in which “U” replaces “T.”

The nucleic acids of this present invention may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages, charged linkages, alkylators, intercalators, pendent moieties, modified linkages, and chelators. Also included are synthetic molecules that mimic polynucleotides in their ability to bind to a designated sequence via hydrogen bonding and other chemical interactions.

The term “nucleic acid” (also referred to as polynucleotide) is also intended to include nucleic acid molecules having an open reading frame encoding a polypeptide, and can further include non-coding regulatory sequences and introns. In addition, the terms are intended to include one or more genes that map to a functional locus. In addition, the terms are intended to include a specific gene for a selected purpose. The gene can be endogenous to the host cell or can be recombinantly introduced into the host cell.

In one aspect the invention also provides nucleic acids which are at least 60%, 70%, 80% 90%, 95%, 99%, or 99.5% identical to the nucleic acids disclosed herein.

The percentage of identity of two nucleic acid sequences or two amino acid sequences can be determined using the algorithm of Thompson et al. (CLUSTALW, 1994, Nucleic Acids Research 22: 4673-4680). A nucleotide sequence or an amino acid sequence can also be used as a so-called “query sequence” to perform a search against public nucleic acid or protein sequence databases in order, for example, to identify further unknown homologous promoters, which can also be used in embodiments of this invention. In addition, any nucleic acid sequences or protein sequences disclosed in this patent application can also be used as a “query sequence” in order to identify yet unknown sequences in public databases, which can encode for example new enzymes, which could be useful in this invention. Such searches can be performed using the algorithm of Karlin and Altschul (1990, Proceedings of the National Academy of Sciences U.S.A. 87: 2,264 to 2,268), modified as in Karlin and Altschul (1993, Proceedings of the National Academy of Sciences U.S.A. 90: 5,873 to 5,877). Such an algorithm is incorporated in the NBLAST and XBLAST programs of Altschul et al. (1990, Journal of Molecular Biology 215: 403 to 410). Suitable parameters for these database searches with these programs are, for example, a score of 100 and a word length of 12 for BLAST nucleotide searches as performed with the NBLAST program. BLAST protein searches are performed with the XBLAST program with a score of 50 and a word length of 3. Where gaps exist between two sequences, gapped BLAST is utilized as described in Altschul et al. (1997, Nucleic Acids Research, 25: 3,389 to 3,402).

As used herein, the term “genetically modified” refers to any change in the endogenous genome of a wild type cell or to the addition of non-endogenous genetic code to a wild type cell, e.g., the introduction of a heterologous gene. More specifically, such changes are made by the hand of man through the use of recombinant DNA technology or mutagenesis. The changes can involve protein coding sequences or non-protein coding sequences, including regulatory sequences such as promoters or enhancers.

The term “recombinant” refers to polynucleotides synthesized or otherwise manipulated in vitro (“recombinant polynucleotides”) and to methods of using recombinant polynucleotides to produce gene products encoded by those polynucleotides in cells or other biological systems. For example, a cloned polynucleotide may be inserted into a suitable expression vector, such as a bacterial plasmid, and the plasmid can be used to transform a suitable host cell. A host cell that comprises the recombinant polynucleotide is referred to as a “recombinant host cell” or a “recombinant bacterium” or a “recombinant cyanobacterium.” The gene is then expressed in the recombinant host cell to produce, e.g., a “recombinant protein.” A “recombinant production gene” refers to a recombinant polynucleotide that encodes a protein involved in the production of a product of interest in the cyanobacterial cell. A recombinant polynucleotide may serve a non-coding function (e.g., promoter, origin of replication, ribosome-binding site, etc.) as well.

The term “homologous recombination” refers to the process of recombination between two nucleic acid molecules based on nucleic acid sequence similarity. The term embraces both reciprocal and nonreciprocal recombination (also referred to as gene conversion). In addition, the recombination can be the result of equivalent or non-equivalent cross-over events. Equivalent crossing over occurs between two equivalent sequences or chromosome regions, whereas nonequivalent crossing over occurs between identical (or substantially identical) segments of nonequivalent sequences or chromosome regions. Unequal crossing over typically results in gene duplications and deletions. For a description of the enzymes and mechanisms involved in homologous recombination see Court et al., “Genetic engineering using homologous recombination,” Annual Review of Genetics 36:361-388; 2002.

The term “non-homologous or random integration” refers to any process by which DNA is integrated into the genome that does not involve homologous recombination. It appears to be a random process in which incorporation can occur at any of a large number of genomic locations.

The term “expressed endogenously” refers to polynucleotides that are native to the host cell and are naturally expressed in the host cell.

The term “operably linked” refers to a functional relationship between two parts in which the activity of one part (e.g., the ability to regulate transcription) results in an action on the other part (e.g., transcription of the sequence). Thus, a polynucleotide is “operably linked to a promoter” when there is a functional linkage between a polynucleotide expression control sequence (such as a promoter or other transcription regulation sequences) and a second polynucleotide sequence (e.g., a native or a heterologous polynucleotide), where the expression control sequence directs transcription of the polynucleotide.

The term “vector” as used herein is intended to refer to a nucleic acid molecule capable of transporting another nucleic acid to which it has been linked. One type of vector is a “plasmid,” which generally refers to a circular double stranded DNA molecule into which additional DNA segments may be ligated, but also includes linear double-stranded molecules such as those resulting from amplification by the polymerase chain reaction (PCR) or from treatment of a circular plasmid with a restriction enzyme.

Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

A “promoter” is an array of nucleic acid control sequences that direct transcription of an associated polynucleotide, which may be a heterologous or native polynucleotide. A promoter includes nucleic acid sequences near the start site of transcription, such as a polymerase binding site. The promoter also optionally includes distal enhancer or repressor elements which can be located as much as several thousand base pairs from the start site of transcription. The term “promoter” is intended to include a polynucleotide segment that can transcriptionally control a gene of interest, e.g., a pyruvate decarboxylase gene that it does or does not transcriptionally control in nature. In one embodiment, the transcriptional control of a promoter results in an increase in expression of the gene of interest. In an embodiment, a promoter is placed 5′ to the gene of interest. A heterologous promoter can be used to replace the natural promoter, or can be used in addition to the natural promoter. A promoter can be endogenous with regard to the host cell in which it is used or it can be a heterologous polynucleotide sequence introduced into the host cell, e.g., exogenous with regard to the host cell in which it is used. Promoters of the invention may also be inducible, meaning that certain exogenous stimuli (e.g., nutrient starvation, heat shock, mechanical stress, light exposure, etc.) will induce the promoter leading to the transcription of the gene.

The term “recombinant nucleic acid molecule” includes a nucleic acid molecule (e.g., a DNA molecule) that has been altered, modified or engineered such that it differs in nucleotide sequence from the native or natural nucleic acid molecule from which the recombinant nucleic acid molecule was derived (e.g., by addition, deletion or substitution of one or more nucleotides). The recombinant nucleic acid molecule (e.g., a recombinant DNA molecule) also includes an isolated nucleic acid molecule or gene of the present invention.

The term “gene” refers to an assembly of nucleotides that encode a polypeptide, and includes cDNA and genomic DNA nucleic acids. “Gene” also refers to a nucleic acid fragment that expresses a specific protein or polypeptide, including regulatory sequences preceding (5′ non-coding sequences) and following (3′ non-coding sequences) the coding sequence.

The term “endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “fragment” refers to a nucleotide sequence of reduced length relative to the reference nucleic acid and comprising, over the common portion, a nucleotide sequence substantially identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least about 6, 50, 100, 200, 500, 1,000, to about 1,500 or more consecutive nucleotides of a polynucleotide according to the invention.

The term “open reading frame,” abbreviated as “ORF,” refers to a length of nucleic acid sequence, either DNA, cDNA or RNA, that comprises a translation start signal or initiation codon, such as an ATG or AUG, and a termination codon and can be potentially translated into a polypeptide sequence.

The term “upstream” refers to a nucleotide sequence that is located 5′ to reference nucleotide sequence. In particular, upstream nucleotide sequences generally relate to sequences that are located on the 5′ side of a coding sequence or starting point of transcription. For example, most promoters are located upstream of the start site of transcription.

The term “downstream” refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription.

The term “homology” refers to the percent of identity between two polynucleotide or two polypeptide moieties. The correspondence between the sequence from one moiety to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments.

As used herein, “substantially similar” refers to nucleic acid fragments wherein changes in one or more nucleotide bases results in substitution of one or more amino acids, but do not affect the functional properties of the protein encoded by the DNA sequence.

The term “substantially similar” also refers to modifications of the nucleic acid fragments of the instant invention such as deletion or insertion of one or more nucleotide bases that do not substantially affect the functional properties of the resulting transcript.

The term “expression”, as used herein, refers to the transcription and stable accumulation mRNA derived from a nucleic acid or polynucleotide. Expression may also refer to translation of mRNA into a protein or polypeptide.

An “expression cassette” or “construct” refers to a series of polynucleotide elements that permit transcription of a gene in a host cell. Typically, the expression cassette includes a promoter and a heterologous or native polynucleotide sequence that is transcribed. Expression cassettes or constructs may also include, e.g., transcription termination signals, polyadenylation signals, and enhancer elements.

The term “codon” refers to a triplet of nucleotides coding for a single amino acid.

The term “codon-anticodon recognition” refers to the interaction between a codon on an mRNA molecule and the corresponding anticodon on a tRNA molecule.

The term “codon bias” refers to the fact that different organisms use different codon frequencies.

The term “codon optimization” refers to the modification of at least some of the codons present in a heterologous gene sequence from a triplet code that is not generally used in the host organism to a triplet code that is more common in the particular host organism. This can result in a higher expression level of the gene of interest.

The term “transformation” is used herein to mean the insertion of heterologous genetic material into the host cell. Typically, the genetic material is DNA on a plasmid vector, but other means can also be employed. General transformation methods and selectable markers for bacteria and cyanobacteria are known in the art (Wirth, Mol Gen Genet. 216:175-177 (1989); Koksharova, Appl Microbiol Biotechnol 58:123-137 (2002). Additionally, transformation methods and selectable markers for use in bacteria are well known (see, e.g., Sambrook et al, supra).

The term “selectable marker” means an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, spectinomycin, kanamycin, hygromycin, and the like.

A “polypeptide” is a polymeric compound comprised of covalently linked amino acid residues. A “protein” is a polypeptide that performs a structural or functional role in a living cell.

A “heterologous protein” refers to a protein not naturally produced in the cell.

An “isolated polypeptide” or “isolated protein” is a polypeptide or protein that is substantially free of those compounds that are normally associated therewith in its natural state (e.g., other proteins or polypeptides, nucleic acids, carbohydrates, lipids).

The term “fragment” of a polypeptide refers to a polypeptide whose amino acid sequence is shorter than that of the reference polypeptide. Such fragments of a polypeptide according to the invention may have a length of at least about 2, 50, 100, 200, or 300 or more amino acids.

A “variant” of a polypeptide or protein is any analog, fragment, derivative, or mutant which is derived from a polypeptide or protein and which retains at least one biological property of the polypeptide or protein. Different variants of the polypeptide or protein may exist in nature. These variants may be allelic variations characterized by differences in the nucleotide sequences of the structural gene coding for the protein, or may involve differential splicing or post-translational modification. The skilled artisan can produce variants having single or multiple amino acid substitutions, deletions, additions, or replacements.

As used herein, the phrase “increased activity” refers to any genetic modification resulting in increased levels of enzyme function in a host cell. As known to one of ordinary skill in the art, enzyme activity may be increased by increasing the level of transcription, either by modifying promoter function or by increasing gene copy number, increasing translational efficiency of an enzyme messenger RNA, e.g., by modifying ribosomal binding, or by increasing the stability of an enzyme, which increases the half-life of the protein, leading to the presence of more enzyme molecules in the cell. All of these represent non-limiting examples of increasing the activity of an enzyme. (mRNA Processing and Metabolism: Methods and Protocols, Edited by Daniel R. Schoenberg, Humana Press Inc., Totowa, N.J.; 2004; ISBN 1-59259-750-5; Prokaryotic Gene Expression (1999) Baumberg, S., Oxford University Press, ISBN 0199636036; The Biomedical Engineering Handbook (2000) Bronzino, J. D., Springer, ISBN 354066808X).

The terms “pyruvate decarboxylase” and “PDC” refer to an enzyme that catalyzes the decarboxylation of pyruvic acid to acetaldehyde and carbon dioxide. A “pdc gene” refers to the gene encoding an enzyme that catalyzes the decarboxylation of pyruvic acid to acetaldehyde and carbon dioxide. The terms “alcohol dehydrogenase” and “ADH” refer to an enzyme that facilitates the interconversion between alcohols and aldehydes or ketones. An “adh gene” refers to the gene encoding an enzyme that facilitates the interconversion between alcohols and aldehydes or ketones, “pdc/adh” refers to the pdc and adh enzymes collectively. A “pdc/adh cassette” refers to a nucleic acid sequence encoding a pdc enzyme and an adh enzyme.

The term “primer” is an oligonucleotide that hybridizes to a target nucleic acid sequence to create a double stranded nucleic acid region that can serve as an initiation point for DNA synthesis under suitable conditions. Such primers may be used in a polymerase chain reaction.

The term “antisense strand” or “antisense sequence” as used herein refers to the single strand DNA molecule of a genomic DNA that is complementary to the sense strand of a gene. The term “antisense” refers to the use of the expression of this sequence to decrease the expression of a target gene in a cell.

The term “knockdown” refers to a partial suppression of the expression of a target gene. A knockdown can occur, for example, when antisense is used. Some expression may still remain when using antisense, so this term is used rather than “knockout”.

The term “knockout” generally refers to a partial or complete suppression of the expression of at least a portion of a protein encoded by an endogenous DNA sequence in a cell. As used herein, the “knockout” relates to the deletion of a target gene.

The term “knockout construct” refers to a nucleic acid sequence that is designed to decrease or suppress expression of a protein encoded by endogenous DNA sequence in a cell. The knockout construct is inserted into a cell, and integrates with the genomic DNA of the cell in such a position so as to prevent or interrupt transcription of the native DNA sequence. Such insertion usually occurs by homologous recombination. The knockout construct nucleic acid sequence may comprise (1) a full or partial sequence of the gene to be suppressed, (2) a full or partial promoter sequence of the gene to be suppressed, or (3) combinations thereof. Typically, the knockout construct is inserted into a cyanobacterial host cell and is integrated into the cell genomic DNA to delete a target gene, usually by the process of homologous recombination.

The phrases “disruption of the gene” and “gene disruption” refer to the deletion or insertion of a nucleic acid sequence into one region of the native DNA sequence and/or the promoter region of a gene so as to decrease or prevent expression of that gene in the cell as compared to the wild-type or naturally occurring sequence of the gene.

The term “inoculum” refers to a volume of cyanobacterial cells that is used to seed a photobioreactor. The inoculum can be, for example, an amount such as 1%, 10%, 30%, or more of the final volume of the photobioreactor. Several rounds of scale up of the inoculum may be required, as in the case of large-scale photobioreactors. The inoculum can be wild type cyanobacteria, or it can be recombinant cyanobacteria. The inoculum can be obtained from a solid plate, a liquid culture, or it can be obtained from cells that were previously frozen (cryostorage).

The term “plurality” means more than one.

The terms “chemical compound of interest” or “product of interest” refer to a product produced by the modified cyanobacteria. In a preferred embodiment, the product is ethanol. The product can be a biofuel, such as bioethanol or biodiesel. The product can also be biomass. The product can be, for example, a pigment (such as beta carotene, astaxanthin, lutein, or other carotenoid pigments). The product can be phycocyanin or related compounds. The product can be a nutraceutical, a fertilizer component, a biofertilizer, or an animal feed. The product can be a chemical compound such as methanol, isopropanol, butanol, isoprene, 1,2-propanediol, 1,3-propanediol, or another suitable chemical that can be made by modified cyanobacteria.

The term “phosphorus” or “P”, as used herein, refers to the chemical element having the atomic number 15. Phosphorus can be in several forms, including phosphate (as the salt potassium phosphate or sodium phosphate), or, for example, as phosphoric acid. A phosphate compound can, for example, be an inorganic phosphate compound such as H₂PO₄⁻, HPO₄²⁻ or PO₄³⁻. The P compound can be, for example, dipotassium phosphate, tripotassium phosphate, or monopotassium phosphate. The P compound can be, for example, sodium dihydrogen phosphate, sodium hydrogen phosphate, or trisodium phosphate. The P compound can be other phosphate salts.

Production of Compounds from Modified Cyanobacterial Cells with Altered Expression of a Phosphate Uptake Regulating Gene

The economic production of chemical compounds of interest with photosynthetically active microorganisms requires large scale outdoor agricultural facilities. The cultivation of cyanobacteria in a sterile and closed system offers the advantage that water with nutrients can be used as cultivation media, without risking nutrient competition with other organisms. But as soon as contaminants, like bacteria and fungi, enter the system, they rapidly grow and can finally lead to a collapse of production. Table 1 lists a variety of bacterial and fungal contaminants which the present inventors detected in outdoor photobioreactors.

TABLE 1
Contaminants found in outdoor cultivations of cyanobacteria
(250 samples in total). Underlined contaminants are
heterotrophic organisms identified as ethanol consumers.

| | | Average | Maximum |
|---|---|---|---|
| Contaminant | Frequency | Abundance | Abundance |
| Bacteria | | | |
| Alphaproteobacteria | | | |
| Labrenzia | 11% | 1.4 × 10⁶ | 9.5 × 10⁶ |
| Nitritireductor | 23% | 3 × 10⁶ | 3 × 10⁷ |
| Oceanicaulis | 63% | 7 × 10⁶ | 7 × 10⁷ |
| Rhizobium | 20% | 8 × 10⁵ | 5 × 10⁶ |
| Sphingomonadaceae | 21% | 9 × 10⁴ | 8 × 10⁵ |
| Thalassospira | 96% | 2 × 10⁶ | 9 × 10⁷ |
| Gammaproteobacteria | | | |
| Alcanivorax | 72% | 8 × 10⁵ | 1.3 × 10⁷ |
| Alteromonas macleodii | 17% | 3 × 10⁵ | 6 × 10⁶ |
| Halomonas | 66% | 3 × 10⁵ | 4.5 × 10⁶ |
| Marinobacter | 19% | 3 × 10⁵ | 1 × 10⁶ |
| Pseudomonas stutzeri | 10% | 2 × 10⁴ | 4 × 10⁴ |
| Bacteroidetes | 15% | 2 × 10⁵ | 1.6 × 10⁶ |
| Firmiticus | <2% | — | — |
| Actinobacteria | <2% | — | — |
| Fungi | 30% | 2 × 10⁵ | 2 × 10⁶ |

In this connection, an unexpected problem identified by the inventors is a frequent appearance of heterotrophic contaminants such as Alcanivorax that use chemical compounds such as ethanol as energy sources and thereby consume the chemical compounds of interest produced by the cyanobacteria for growth and proliferation. The present inventors further determined that these heterotrophic contaminants can cause a devastating loss of the chemical compound of interest in the cyanobacterial culture. It was thus concluded that solving the problem of improving production yield and economic efficiency of cyanobacterial production of chemical compounds of interest requires suppression of the growth of ethanol consuming contaminants in the cyanobacterial cultures.

The present invention is based on the idea that growth of contaminants can be delayed, reduced or prevented by limiting nutrient availability to contaminants. The vulnerability of contaminants to phosphate starvation was determined as a particularly suitable attack point for effective microbial control, when at the same time a complementary genetically modified cyanobacterial cell with improved competitiveness for phosphate is provided.

As used herein, a protein involved in regulation of phosphate metabolism refers to any protein which directly or indirectly influences cellular uptake and/or intracellular storage of the phosphate compound in the cyanobacterial cell. In general, this can be a positive regulation or a negative regulation. For example, the regulation can affect phosphate transport and/or relate to mechanisms by which the cyanobacterial cell senses and responds to phosphate limitation. Exemplary regulations include transcriptional regulation and/or translational regulation, a signal transduction pathway, post-translational regulation, allosteric regulation, and combinations thereof.

An increased cellular uptake of the phosphate compound can, for example, be determined by comparing the phosphate transfer across the cell membrane between the recombinant cyanobacterial cell and a control cell not harboring the genetic modification, when both are cultured under the same conditions. Increased cellular storage of the phosphate compound can, for example, be determined by comparing the abundance of particulate phosphate, i.e. intracellular phosphate, in the recombinant cyanobacterial cell and a control cell cultured under the same conditions. Suitable methods for determining these parameters are described in Example 5 below.

The improvement of cellular uptake and/or intracellular storage of phosphate in the cyanobacterial cell provides the cells with a competitive growth advantage over concomitant, co-existing microorganisms. In this way, growth of contaminants which negatively influence the economic production of chemical compounds of interest can be significantly reduced.

Preferably, the phosphate uptake regulating gene whose expression is altered is endogenous to the cyanobacterial cell. As used herein, “endogenous” refers to elements such as DNA sequences or proteins that originate from within a native form of the cyanobacterial cell. Conversely, “exogenous” refers to such elements that are present in a cyanobacterial host cell but that originated outside of that host cell. The inventors found that a genetic modification that alters expression of an endogenous phosphate uptake regulating gene provides particular benefits, because it changes regulation of the cell's natural phosphate metabolism. In particular, the inventors avoid unnecessarily stressing the cell by heterologous expression of additional proteins to enhance phosphate uptake.

In a preferred variant, the protein involved in regulation of phosphate metabolism regulates expression and/or activity of at least one phosphate transport protein involved in transfer of the phosphate compound from an external solution into the cyanobacterial cell. In this way, the inventors achieve an enhanced uptake speed and/or affinity for the available phosphate compound.

It is possible that the protein involved in regulation of phosphate metabolism is a positive regulator, i.e. the protein that activates or enhances expression and/or activity of the phosphate transport protein in the cyanobacterial cell in its native form. Alternatively, the protein involved in regulation of phosphate metabolism can be a negative regulator, i.e. a protein that represses expression and/or activity of the phosphate transport protein in the cyanobacterial cell in its native form. Preferably, the protein involved in regulation of phosphate metabolism is a negative regulator. In this way, the inventors change a natural phosphate response mechanism of the cell to mimic a physiological state of permanent phosphate starvation. This allows the decoupling of phosphate uptake from the actual environmental phosphate availability.

For example, it is possible that the genetic modification causes a constitutive expression and/or activity of the at least one phosphate transport protein. In this way, the inventors achieve a continuous (over-)expression of the phosphate transport protein, so that the cyanobacterial cell of the present invention quickly takes up the phosphate compound at any time during cultivation. At the same time, with the negative phosphate regulation disabled, the cyanobacterial cell is also able to take up and store the phosphate compound beyond its metabolic needs. Thus, these characteristics act synergistically in enhancing the competitiveness of the cyanobacterial cell for the phosphate compound over heterotrophic contaminants in the culture.

The at least one phosphate transport protein can form part of a phosphate transport complex that includes additional proteins. Preferably, the at least one phosphate transport protein and/or additional protein is selected from a group consisting of PstS, PstC, PstA, PstB, and combinations thereof. These proteins form the high affinity uptake system PstSCAB which plays a key role in transferring inorganic phosphate into the cyanobacterial cell. The proteins are encoded by the pstSCAB operon which is endogenous to many cyanobacteria such as Cyanobacterium sp. PTA-13311.

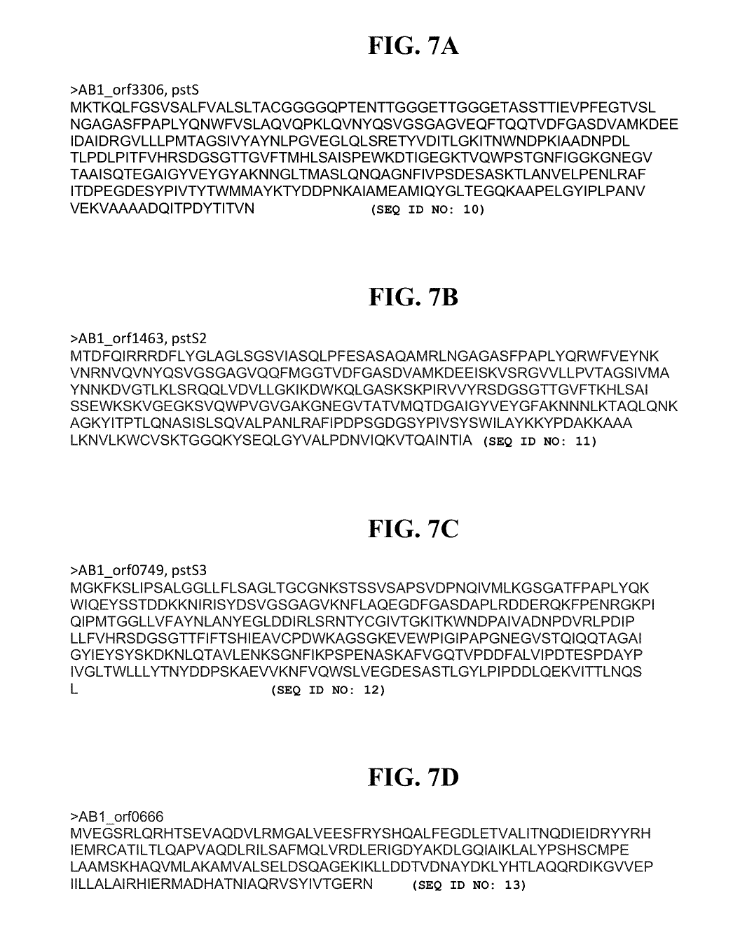

In particular, the at least one phosphate transport protein comprises a phosphate binding protein capable of binding the phosphate compound. More particularly, the phosphate transport protein comprises an amino acid sequence having at least 16 identical residues without gaps to a consensus sequence VNYQSVGSGAGLRQFIXGTVDFAGSDLPL (SEQ ID NO: 41), wherein X denotes any one of the 20 natural amino acids. The phosphate transport protein can also comprise an amino acid sequence having at least 80%, at least 90% or at least 95% sequence identity with any one of the sequences pstS, pstS2 or pstS3 shown in FIG. 7A to 7C. The percentage of identity of two nucleic acid sequences or two amino acid sequences can for instance be determined using the algorithm of Thompson et al. (ClustalW, Nucleic acid Research 22, 1994, 4673-4680). A nucleotide sequence or an amino acid sequence can also be used as a so-called query sequence to perform a nucleic acid or amino acid sequence search against public nucleic acid or protein sequence databases in order to, for example, identify further homologous protein sequences and/or nucleic acid sequences which can also be used in embodiments of this invention. In addition, any nucleic acid sequence or protein sequence disclosed in this patent application can also be used as a query sequence in order to identify yet unknown sequences in public databases, which can encode for example new enzymes which could be useful in this invention. Such searches can be performed using the algorithm of Karlin and Altschul (Proceedings of the National Academy of Sciences, USA, 1990, 87; pages 2264 to 2268), modified as in Karlin and Altschul (Proceedings of the National Academy of Sciences, USA, 1993, 90; pages 5873 to 5877). Such an algorithm is incorporated in the BLASTN and BLASTX programs of Altschul et al. (Journal of Molecular Biology 1990, 215, pages 403 to 410). Suitable parameters for these database searches with these programs are, for example, a score of 100 and a word length of 12 for BLAST nucleotide searches as performed with the BLASTN program. BLAST protein searches are performed with the BLASTX program with a score of 50 and a word length of 3. Where gaps exist between two sequences, the gapped BLAST is utilized as described in Altschul et al. (Nucleic Acid Research, 1997, 25: pages 3389 to 3402).

By up-regulating the expression of these proteins, the inventors accomplished not only an accelerated uptake of the phosphate compound into the cyanobacterial cell, but also lowered the threshold concentration down to which the phosphate compound can still be transferred from the medium into the cyanobacterial cell. In contrast to a conventional cyanobacterium, the cyanobacterial cell of the present invention is thus enabled to rapidly and almost completely deplete the phosphate compound from the medium, thereby leaving insufficient phosphate compound available in the medium for contaminant growth.

The genetic modification that alters expression of the at least one phosphate uptake regulating gene can be any change in the endogenous genome of the native form of the cyanobacterial cell. Such genetic modification can include the addition of endogenous and non-endogenous, exogenous genetic code to the native form of the cyanobacterial cell. More specifically, the genetic modification is made by the hand of man through the use of recombinant DNA technology or mutagenesis. The modification can involve protein coding sequences or non-protein coding sequences in the genome such as regulatory sequences, non-coding RNA, antisense RNA, promoters or enhancers. Aspects of the invention therefore utilize techniques and methods common to the fields of molecular biology, microbiology and cell culture. Useful laboratory references for these types of methodologies are readily available to those skilled in the art. See, for example, “Molecular Cloning: a Laboratory Manual” (third edition), Sambrook, J., et al. (2001) Cold Spring Harbor Laboratory Press; “Current Protocols in Microbiology” (2007), Coico, R. et al. (eds.), John Wily & Sons, Inc.; “The Molecular Biology of Cyanobacteria” (1994), Bryant, D. (ed.), Springer Netherlands; “Handbook of Microalgal Culture; Biotechnology and Applied Phycology” (2003), Richmond, A. (ed.), Blackwell Publishing; and “The Cyanobacteria, Molecular Biology, Genomics and Evolution”, Herrero, A. and Flores, E. (eds.), Caister Academic Press, Norfolk, UK, 2008. These publications are hereby used to describe more fully the state of the art to which this invention pertains.

One possibility is that the genetic modification comprises a heterologous nucleic acid sequence encoding a knockdown component that reduces or eliminates the expression of the phosphate transport regulating protein. As used herein, the term “heterologous” refers to an element such as a gene, part of a gene or protein in a cyanobacterium which does not naturally have this element. For example, a “heterologous nucleic acid sequence” has been inserted into the host organism by recombinant DNA technology. The term “heterologous” also means a DNA sequence which appears endogenously in the cyanobacterium but is additionally present in a non-native form, for instance by forming part of a synthetic plasmid or by artificially controlling expression of the DNA sequence by a promoter which is not naturally controlling the sequence in the cyanobacterium.

The knockdown component can comprise RNA transcribed from the heterologous nucleic acid that is at least partially complementary to mRNA transcribed from the phosphate uptake regulating gene for binding to the mRNA and initiating degradation and/or inhibiting translation of at least part thereof. For example, the heterologous nucleic acid can encode a small RNA (sRNA) or an antisense RNA (asRNA) to silence the expression of the phosphate uptake regulating gene.

The knockdown component can also comprise sgRNA adapted to bind to the phosphate uptake regulating gene and/or dCAS9 for repressing expression of the phosphate uptake regulating gene on the transcriptional level by CRISPRi. Another possibility is that the knockdown component comprises a transcription activator like effector nuclease (TALEN) adapted to bind to the phosphate uptake regulating gene and initiating degradation or inhibiting transcription of at least part thereof.

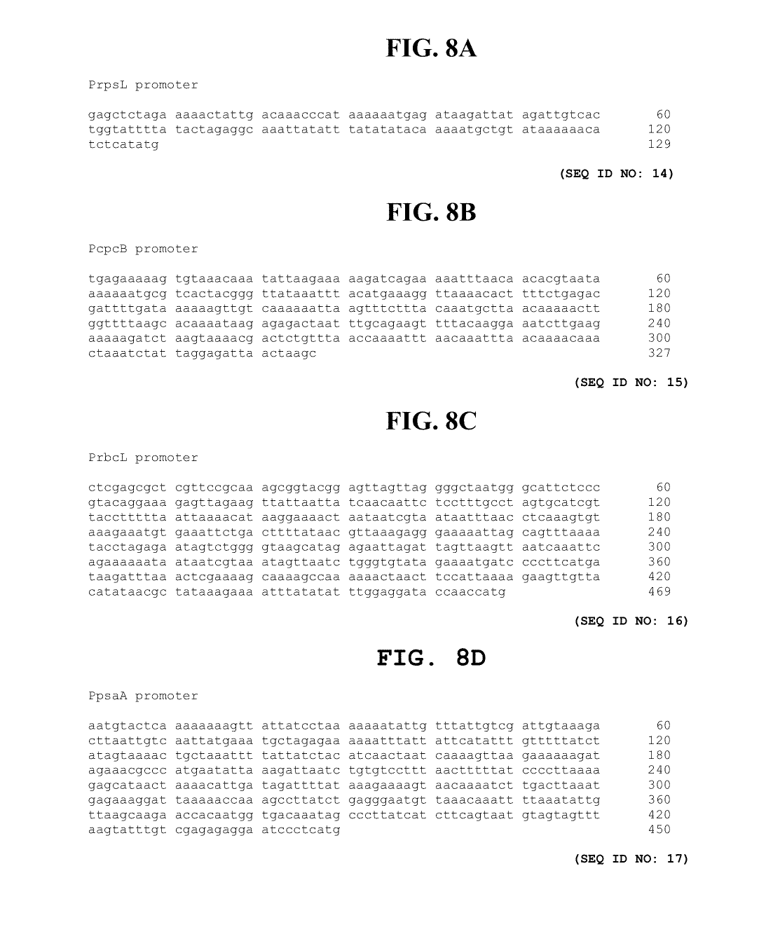

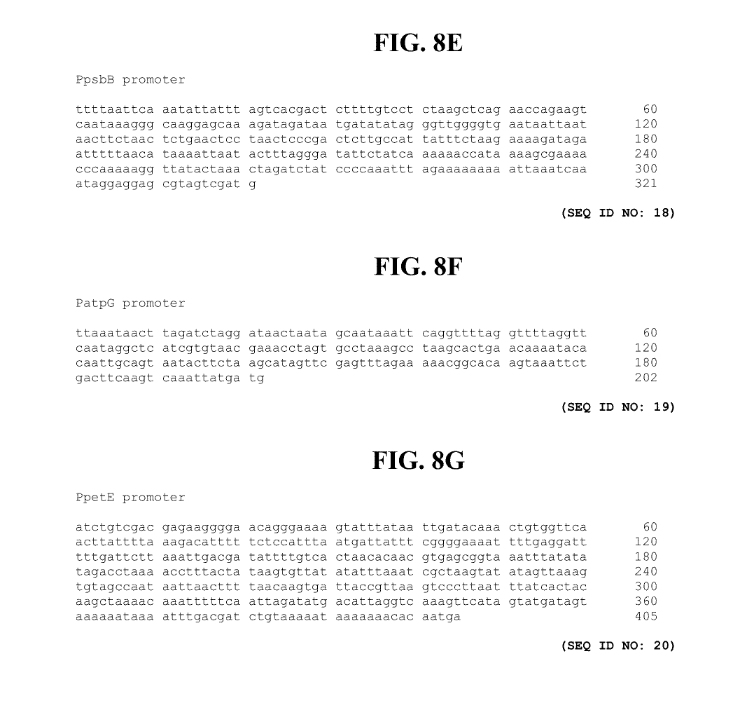

The expression of the knockdown component is preferably controlled by a constitutive promoter or, as the case may be, a promoter that is at least constitutive under typical cyanobacterial culturing conditions. Suitable constitutive promoters for the aspects of the present invention include, but are not limited to, PrpsL, PcpcB, PrbcL, PpsaA, PpsbB, PatpG, and variations thereof. Accordingly, the constitutive promoter can comprise a nucleotide sequence having at least 80% sequence identity, at least 90% sequence identity or at least 95% sequence identity with any one of the nucleotide sequences shown in FIG. 8A-F. Still suitable is the copper-regulated PpetE promoter or a promoter comprising a nucleotide sequence having at least 80% sequence identity, at least 90% sequence identity or at least 95% sequence identity with the nucleotide sequences shown in FIG. 8G.

Alternatively, the genetic modification can comprise at least partial disruption or complete removal of the phosphate uptake regulating gene. In this way, the gene may be translated into a protein which has an altered or reduced function or is non-functional. Preferably, the gene is not translated at all. It is possible that the genome of the cyanobacterial cell harbors more than one copy of the phosphate uptake regulating gene. In such a case, it is further preferred that all copies of the gene comprise the at least partial disruption or, more preferably, have been completely removed in order to deprive the cyanobacterium of the possibility to restore the phosphate uptake regulating gene. Even a restoration by homologous recombination is unlikely, in particular after complete deletion of the gene.

Preferably, the protein involved in regulation of phosphate metabolism comprises PhoU or a homolog or analog thereof. Accordingly, the protein preferably contains a full length match with at least 8 out of 13 identities to the amino acid sequence DLERIGDLAXNIA (SEQ ID NO: 42), wherein X is any one of the 20 natural amino acids, and/or a full length match with at least 9 out of 13 identities to the amino acid sequence LERIGDHATNIAE (SEQ ID NO: 43). The protein can also comprise an amino acid sequence having at least 80%, at least 90% or at least 95% sequence identity with the amino acid sequence encoded by orf0666 shown in FIG. 7D. PhoU is a global phosphate response regulator which is responsible for the repression of the high affinity PstSCAB uptake system in cyanobacteria and also regulates expression of other genes involved in the response to phosphate limitation. With this key player in cyanobacterial phosphate metabolism made to be non-functional, the inventors did not only accomplish a more than threefold faster uptake of the phosphate compound with the cyanobacterial cell of the present invention, but also surprisingly observed an intracellular polyphosphate storage which was more than a doubled in comparison to a conventional cyanobacterial cell.

The phosphate compound can comprise an inorganic phosphate compound, typically an orthophosphate, such as dihydrogen phosphate H₂PO₄⁻, hydrogen phosphate HPO₄²⁻ and/or PO₄³⁻.

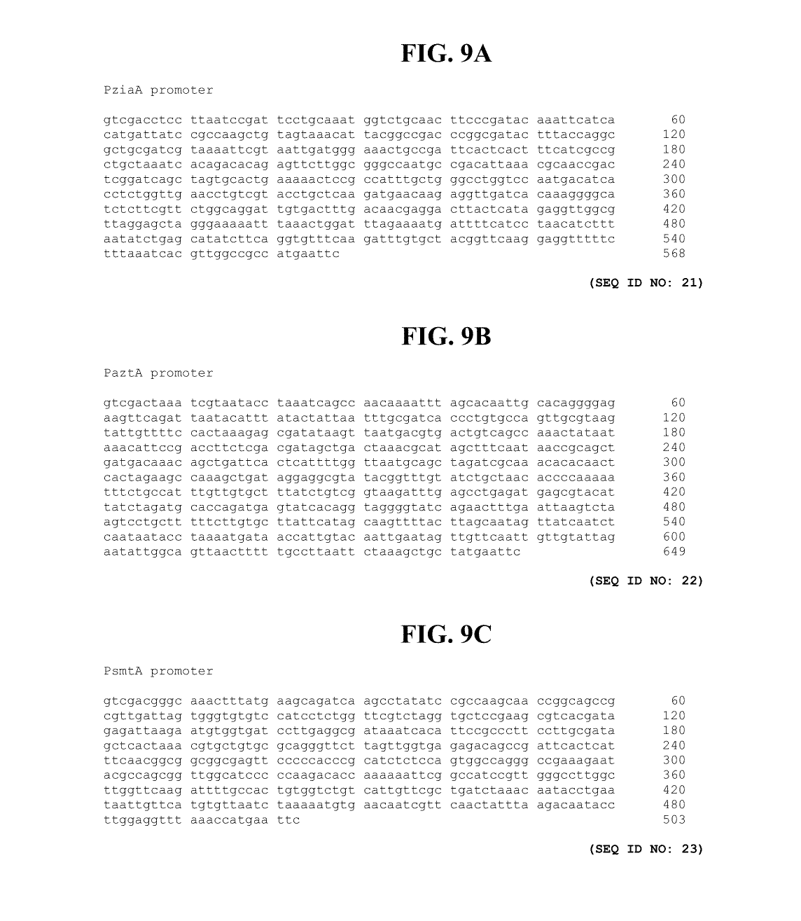

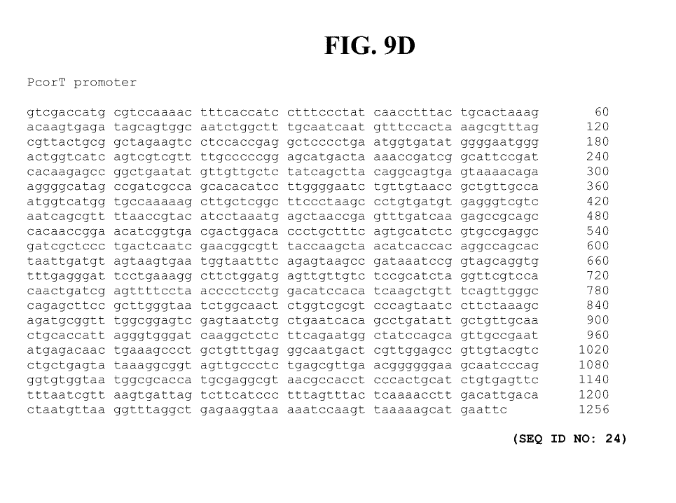

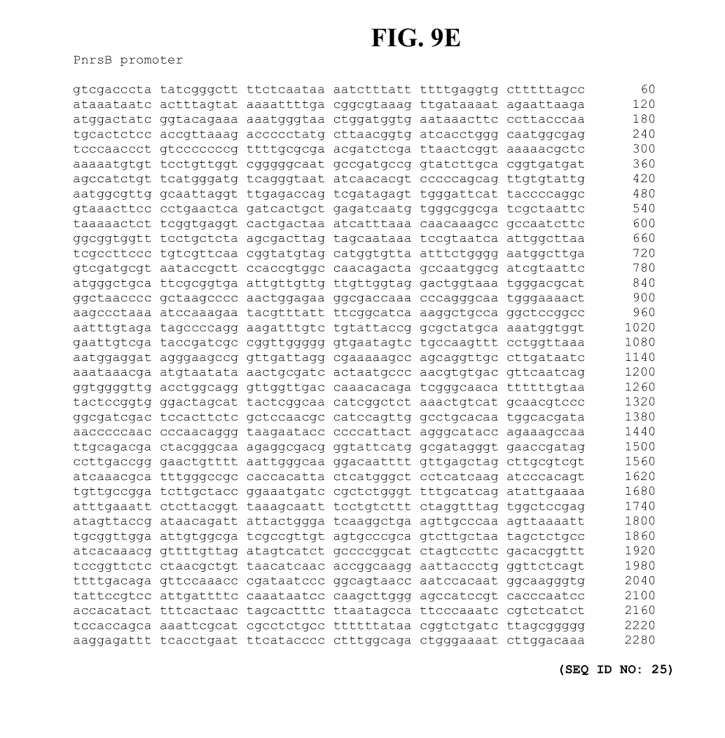

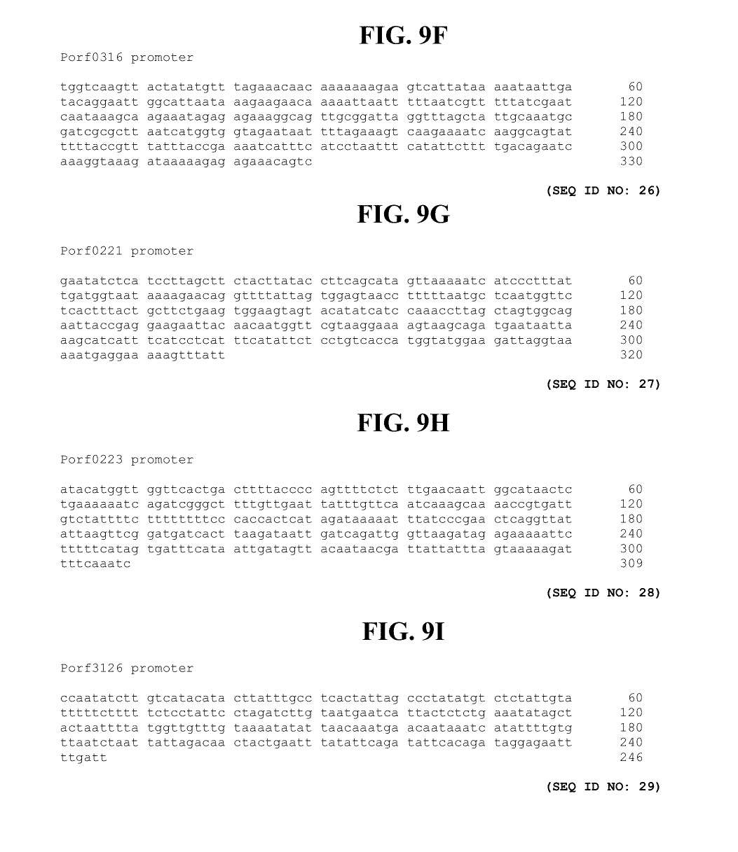

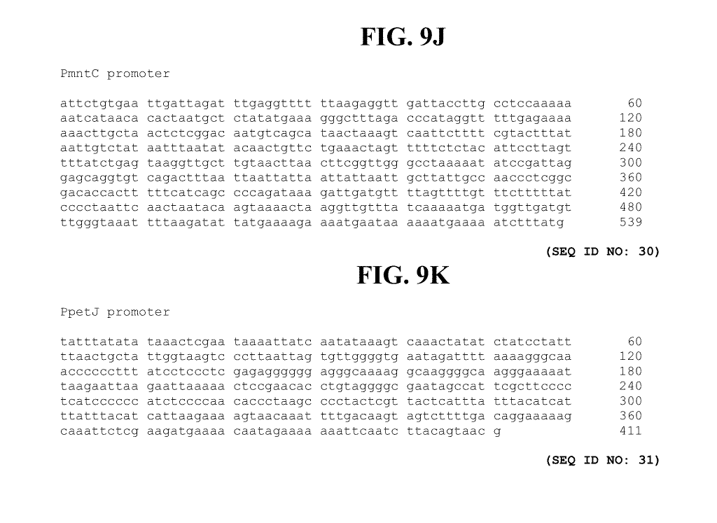

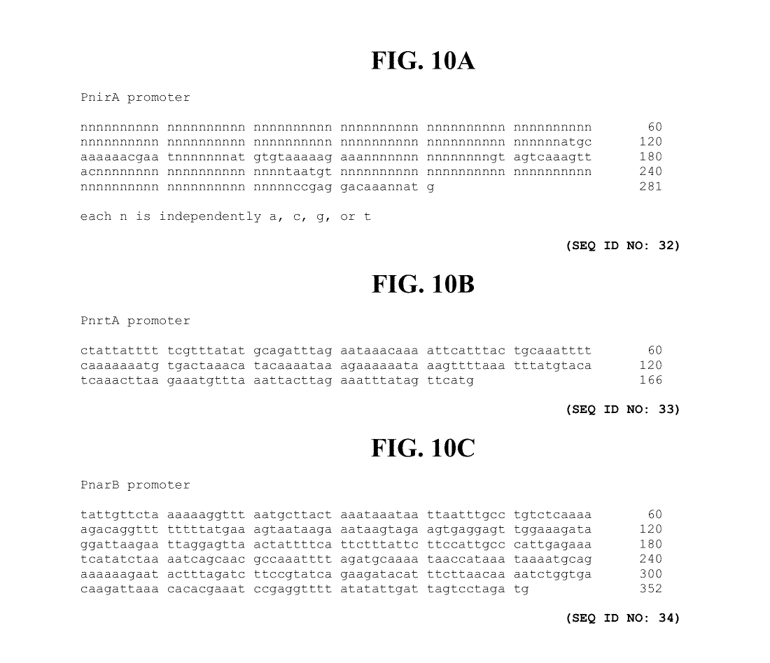

The production gene can be controlled by an inducible promoter or a constitutive promoter. Suitable inducible promoters include, but are not limited to, promoters inducible by a change of metal ion concentration. Preferably, the inducible promoter is selected from a group comprising PziaA, PaztA, PsmtA, PcorT, PnrsB, Porf0316, Porf0221, Porf0223, Porf3126, PmntC, PpetJ, and variations thereof. Accordingly, the inducible promoter can comprise a nucleotide sequence having at least 80% sequence identity, at least 90% sequence identity or at least 95% sequence identity with any one of the nucleotide sequences shown in FIG. 9A-K. Alternatively, the inducible promoter can be nitrate inducible. Preferably, the nitrate inducible promoter is selected from a group consisting of PnirA, PnrtA, PnarB, and variations thereof. Accordingly, the nitrate-inducible promoter can comprise a nucleotide sequence having at least 80% sequence identity, at least 90% sequence identity or at least 95% sequence identity with any one of the nucleotide sequences shown in FIG. 10A-C.

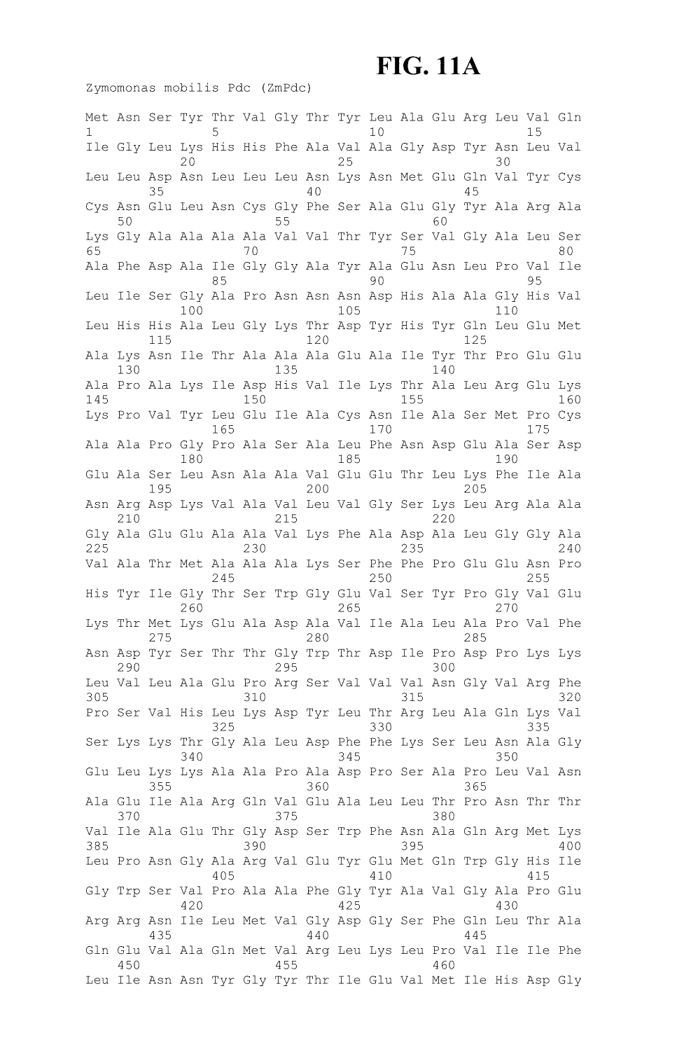

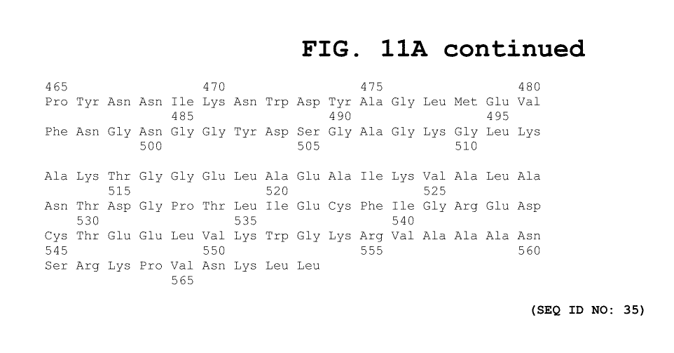

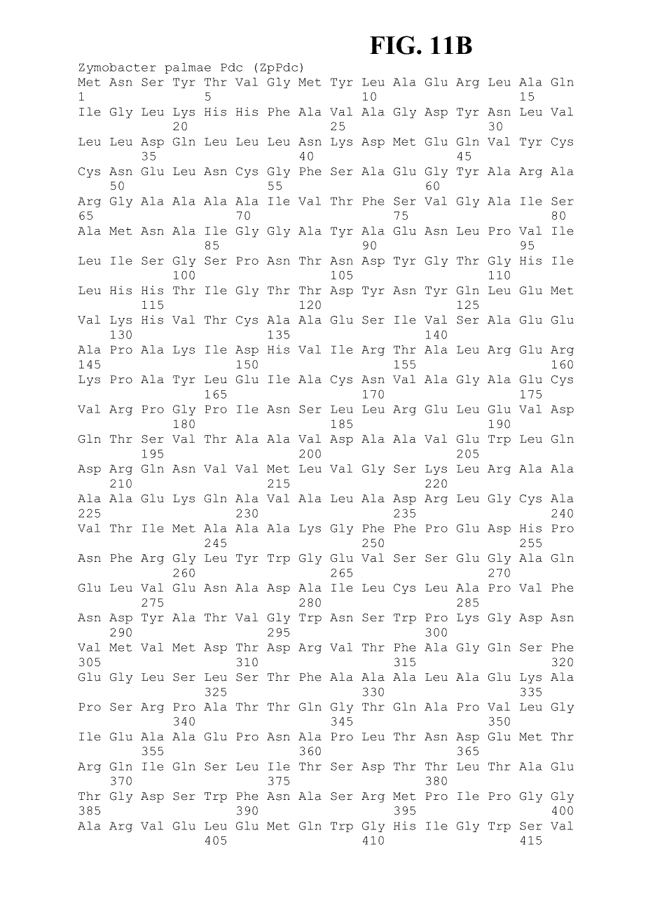

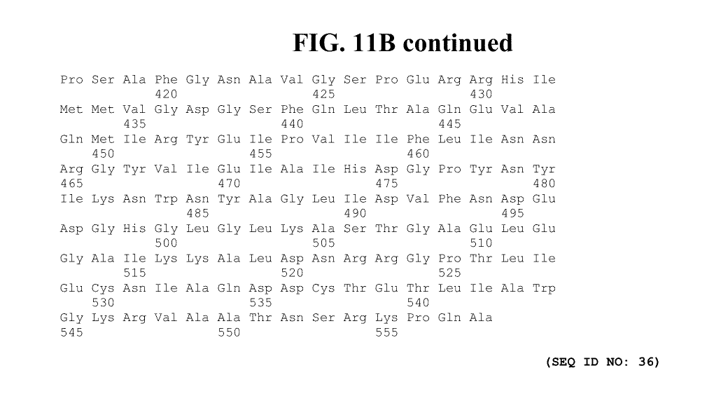

The enzyme for the production of the chemical compound of interest preferably comprises a pyruvate decarboxylase (Pdc). The pyruvate decarboxylase enzyme preferably comprises an amino acid sequence with at least 80% sequence identity, preferably at least 90% sequence identity and most preferably at least 95% sequence identity with the amino acid sequence of ZmPdc (FIG. 11A) or ZpPdc (FIG. 11B). An inducible promoter preferably controls expression of the Pdc.

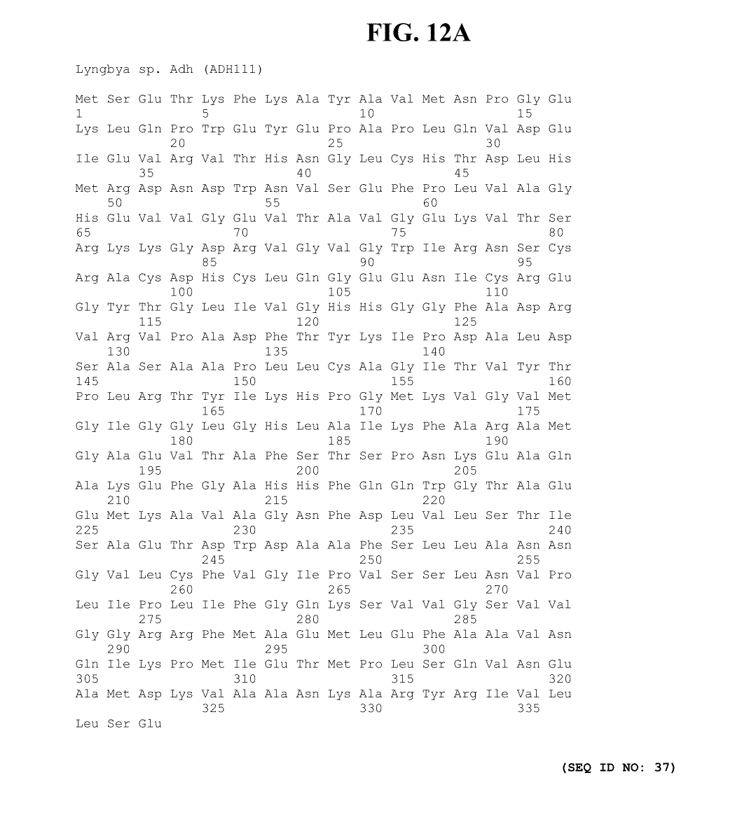

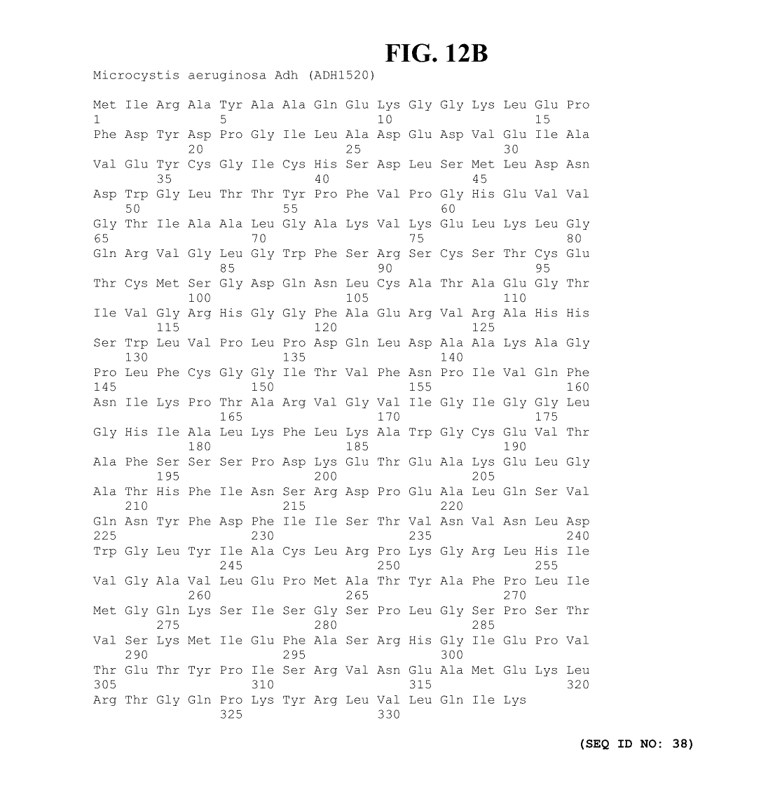

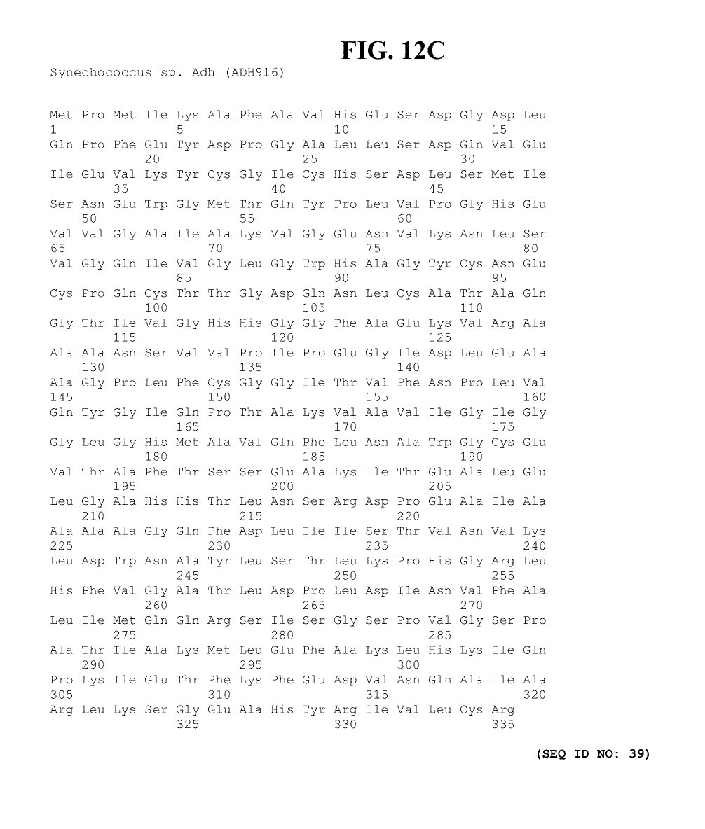

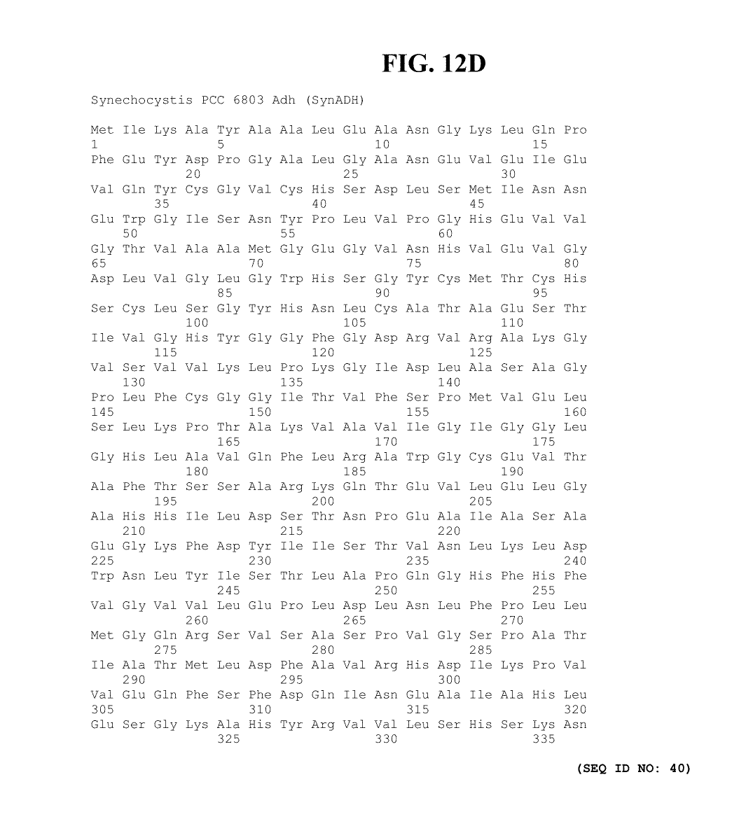

It is also possible that additional recombinant production genes are present, for example a recombinant gene encoding an alcohol dehydrogenase enzyme (Adh). Preferred alcohol dehydrogenase enzymes comprise a nucleotide sequence having at least 80%, at least 90% or at least 95% sequence identity with any one of the amino acid sequence of ADH111, ADH1520, ADH916 or SynAdh shown in FIG. 12A-D. The recombinant gene encoding the alcohol dehydrogenase enzyme is preferably under transcriptional control of a constitutive promoter. Suitable constitutive promoters correspond to those already mentioned above.

It is also possible that both the recombinant genes encoding Pdc and Adh are included in a single operon controlled by a single promoter. An inducible promoter preferably controls expression of the operon including the recombinant genes encoding Pdc and Adh.

Preferably, the chemical compound of interest is ethanol. However, the cyanobacterium can also be metabolically enhanced to produce other compounds of interest, including but not limited to, isopropanol, isoprene, 1,2-propanediol, 1,3-propanediol or n-propanol.

Exemplary products of interest include, but are not limited to, organic carbon compounds, alcohols, fatty acids, oils, carotenoids, proteins, colorants or pigments such as phycocyanin, enzymes, biofuels, biomass, nutraceuticals, beauty products such as lotions, skin products, and hair products, vitamins, pharmaceuticals, and the like.

In an embodiment, the product of interest is a colorant, such as phycocyanin, phycobiliprotein, or a derivative thereof. Other colorants of interest can be, for example, allophycocyanin, phycoerythrin, or phycoerythrocyanin, and the like. The genes for the production of the colorant can be endogenous to the cyanobacterial cell, or can be exogenously derived.

The product of interest can also be a pigment such as a carotenoid. The genes for the production of a carotenoid of interest can be endogenous to the cyanobacterial cell, or can be exogenously derived. Exemplary carotenoids that can be obtained from cyanobacterial cultures include, but are not limited to, lutein, zeaxanthin, beta-carotene, astaxanthin, canthaxanthin, and the like.

In another embodiment, the product of interest is an industrial enzyme. The enzyme can be endogenous to the cell or can be exogenously derived. An exogenously derived gene encoding an enzyme of interest can be inserted into the plasmid vector or integrated into the genome of the cyanobacteria. A culture of the cells is grown, the cells are harvested, and the enzyme of interest is isolated and purified.

The cyanobacterial cell of the present invention can be selected from the group consisting of Synechocystis, Synechococcus, Anabaena, Chroococcidiopsis, Cyanothece, Lyngbya, Phormidium, Nostoc, Spirulina, Arthrospira, Trichodesmium, Leptolyngbya, Plectonema, Myxosarcina, Pleurocapsa, Oscillatoria, Pseudanabaena, Cyanobacterium, Geitlerinema, Euhalothece, Calothrix, and Scytonema. Particularly preferred is Cyanobacterium sp. deposited in the ATCC under the accession number PTA-13311, also termed AB1 herein.

In a second aspect, this invention provides a method for producing a chemical compound of interest with the recombinant cyanobacterial cell described above. The method comprises (A) culturing the cyanobacterial cell in a phosphorus compound-poor medium containing from ≧0 to 100 μM of the phosphorus compound, the cyanobacterial cell expressing the enzyme for the production of the chemical compound of interest, thereby producing the chemical compound of interest.

The present method complements the physiological advantages of the recombinant cyanobacterial cell in suppressing growth of contaminants and preventing heterotrophic consumption of the chemical compound of interest in the culture. The faster phosphate uptake and higher intracellular phosphate storage capability of the recombinant cyanobacterial cell of the present invention allowed the inventors to tightly limit phosphate availability in the culture. For example, the medium can be essentially free of the phosphate compound if the recombinant cyanobacterial cell already contains a high intracellular level of the phosphate compound. It is also possible that the medium contains the phosphate compound in a low concentration that covers just about the cyanobacterial cell's demand for phosphate, for example 0.1 to 100 μM or 1 to 100 μM, the phosphate compound being rapidly depleted from the medium by the recombinant cyanobacterial cell. In either case, the lack of phosphate compound in the medium is very effective in preventing up-growth of contaminants. Surprisingly, the tight phosphate limitation does not negatively influence the cell growth and productivity of the recombinant cyanobacterial cell of the present invention in comparison to a conventional ethanol-producing cyanobacterial cell.

The present method is particularly advantageous when step (A) comprises a non-axenic culturing condition, for instance when at least one heterotrophic microorganism is present in the cyanobacterial culture. The phosphate limitation efficiently suppresses growth of such contaminants and their consumption of the chemical compound, so that typically the loss of the chemical compound of interest caused by the heterotrophic microorganism is maintained below 20% of the total amount of the chemical compound of interest produced during step (A). As used herein, a loss includes heterotrophic consumption of the chemical compound of interest as an energy source, as well as a chemical modification made by a contaminating microorganism on the chemical compound of interest.

In a preferred variant, the method further comprises prior to step (A) the step (A′) culturing the cyanobacterial cell in a phosphate compound-rich medium which contains more than 100 μM of the phosphate compound, so that the cyanobacterial cell takes up the phosphate compound in (A′) before the cultivation is continued in the phosphate compound-poor medium in (A). Due to the enhanced phosphate compound uptake and storage capability of the recombinant cyanobacterial cell, step (A′) results in an efficient preloading of the cyanobacterial cell with the phosphate compound. This preloading with the phosphate compound in (A′) is sufficient to sustain the phosphate requirements of the cyanobacterial cell in the presence of very low concentrations or even the complete absence of the phosphate compound in the medium during the subsequent culturing step (A), when at the same time up-growth of contaminants suffers from phosphate starvation under these conditions.

The phosphate compound rich medium can also contain more than 200 μM, more than 300 μM, more than 500 μM, more than 700 μM, more than 1,000 μM or even more than 2,500 μM of the phosphate compound. Preferred concentrations of the phosphate compound in the phosphate compound-rich medium during (A′) also include at least 35 μM, 100 μM, 200 μM, 250 μM, 500 μM or 1,000 μM phosphate compound per OD₇₅₀ₙₘ of the culture.

It is possible that the concentrations specified above are available in the phosphate compound-rich medium only once and/or only momentarily or for a short time, or are present at multiple times, for example by adding multiple doses of the phosphate compound so that the culture contains the given concentrations. The same applies to the concentrations specified for the phosphate compound-poor medium.

The phosphate compound-rich medium in (A′) and the phosphate compound-poor medium in (A) can be provided as separate media. In this variant, the method typically comprises transferring the recombinant cyanobacterial cell from the phosphate compound-rich medium in (A′) to the phosphate compound-poor medium in (A). Alternatively, the phosphate compound-poor medium of (A) can, at least partially, originate from the phosphate compound-rich medium. For example, the phosphate compound-poor medium can comprise a dilution of the phosphate compound-rich medium. It is also possible that the method step (A′) comprises depletion of the phosphate compound from the phosphate compound-rich medium by the recombinant cyanobacterial cell, thereby resulting in the phosphate compound-poor medium. Then, (A) comprises continuing culturing the recombinant cyanobacterial cell in the so-obtained phosphate compound-poor medium resulting from the depletion.

Owing to its improved phosphate compound uptake and storage capabilities, the cyanobacterial cell of the present invention quickly reaches its maximum phosphate compound loading capacity, so that method step (A′) can be kept surprisingly short. For example, (A′) can comprise culturing the cyanobacterial cell in the phosphate compound-rich medium for 1 to 72 hours, preferably for 12 to 48 hours. This short phosphate loading period is beneficial for the contamination control, since outgrowth of contaminants in the phosphate compound-rich medium is significantly limited in such a short time. If a conventional cyanobacterial cell requiring a longer loading period in (A′) was used, there would be a risk that contaminants could be carried over to the production phase in method step (A).

Another possibility is to maintain cultivation of the cyanobacterial cell in (A′) until the cyanobacterial cell has at least partially depleted the phosphate compound from the phosphate compound-rich medium, for example until at least 50%, 90%, 95%, 99% or 99.9% of the phosphate compound have been depleted from the phosphate compound-rich medium. In this way, carryover of the phosphate compound between method steps (A′) and (A), which may occur for example by transferring and/or diluting the cyanobacterial cell of step (A′) in the phosphate compound-poor medium of step (A), is avoided. In a conventional method, such phosphate carryover may otherwise trigger contaminant growth during the production phase.

In some cases, it can be beneficial to feed the cyanobacterial cells with additional phosphate compound during the production phase.

Accordingly, method step (A) can further comprise at least one addition of the phosphate compound to the phosphate compound-poor medium during culturing of the cyanobacterial cell. For example, such an addition can comprise from 1 to 100 μM of the phosphate compound per culture volume. Preferred concentrations of the phosphate compound addition can range from 10 to 75 μM per culture volume. Since the recombinant cyanobacterial cells of the present invention take up the phosphate compound better and faster than the contaminants, the added phosphate can be immediately taken up by the cyanobacteria and essentially no dissolved phosphate compound is left in the medium to support contaminant growth. It is also possible that method step (A) comprises multiple additions of the phosphate compound in the specified concentrations, each for example in the range of 10 μM to 40 μM per culture volume. In an embodiment, the multiple additions are daily additions.

Nevertheless, the improved uptake and storage capability of the cyanobacterial cell of the present invention allows one to maintain method step (A) for at least two days before the first addition of a phosphate compound is made. In some cases, the first addition of the phosphate compound in method step (A) can be even further delayed. For example, method step (A) can be maintained for at least four, five, seven, ten or fourteen days before the first addition of the phosphate compound to the phosphate compound-poor medium is made.

Preferably, the recombinant production gene encoding the enzyme for the production of the chemical compound of interest is under the transcriptional control of an inducible promoter which can be induced by an exogenous stimulus, and the exogenous stimulus is provided or enhanced in method step (A).

Typically, the cyanobacterium is exposed to light and CO₂ during method steps (A) and (A′).

In a third aspect, the present invention provides use of the cyanobacterial cell described above in a cyanobacterial culture for reducing or preventing loss of the produced chemical compound of interest due to consumption by heterotrophic microorganisms. In particular, growth of the heterotrophic microorganisms is reduced or inhibited by limiting the availability of the phosphate compound in the cyanobacterial culture. Accordingly, the culturing conditions and phosphate compound feeding regimes of the second aspect are also applicable to the third aspect of the present invention.

Finally, in a fourth aspect, this invention provides a method for producing the cyanobacterial cell described above. The method comprises A) providing at least one transformable nucleic acid construct for the genetic modification that alters expression of the at least one phosphate uptake regulating gene encoding the protein involved in regulation of phosphate metabolism and at least one transformable nucleic acid construct comprising the at least one recombinant production gene encoding the enzyme for the production of the chemical compound of interest, and B) transforming the transformable nucleic acid constructs into a cyanobacterial cell.

In the following, certain embodiments of the invention will be explained in more detail with reference to figures and experimental data. The figures and examples are not intended to be limiting with respect to specific details.

### EXAMPLES

### Example 1

### General Cultivation Conditions

Cyanobacterial cells were grown in BG11/BG11⁰ medium or mBG11/mBG11⁰ medium in different scales. The recipe used for the cyanobacterial growth medium BG11 and mBG11 was:
NaNO₃: 1.5 g (NaNO₃ was omitted in media designated “BG11⁰”)
MgSO₄.7H₂O: 0.075 g
CaCl₂.2H₂O: 0.036 g
Citric acid: 0.006 g
Ferric ammonium citrate: 0.006 g
EDTA (disodium salt): 0.001 g
NaCO₃: 0.02 g

Unless differently specified hereinafter, the standard medium further contained 0.04 g K₂HPO₄.
Trace metal mix A5: 1.0 ml
For artificial seawater mBG11, the following further salts were added:
   NaCl: 25.84 g
   MgSO₄.7H₂O: 6.36 g
   MgCl₂.6H₂O: 5.06 g
   KCl: 0.62 g
CaCl₂.2H₂O: 1.36 g
Distilled water: ad 1.0 L

Herein, the recipe for the trace metal mix A5 was:
H₃BO₃: 2.86 g
MnCl₂.4H₂O: 1.81 g
*ZnSO₄.7H₂O: 0.222 g
NaMoO₄.2H₂O: 0.39 g
*CuSO₄.5H₂O: 0.079 g
*Co(NO₃)2.6H₂O: 49.4 mg
Distilled water: ad 1.0 L

The asterisk (*) denotes those metal supplements that can be either temporarily omitted or used in reduced amounts if these metals are also used as inductor for corresponding metal-inducible promoters in the recombinant cyanobacterial strain.

### Example 2

### Construction of Ethanologenic Plasmid Vectors

The plasmid vectors used in this work are presented in the sections “Brief description of the several views of the drawings”. Denominations of genes are presented in a three letter lower case name which can be followed by a capitalized letter or number if more than one related gene exists. The respective protein encoded by that gene is denominated by the same name with the first letter capitalized.

Denominations for promoter sequences, which control the transcription of a certain gene in their natural environment are given by a capitalized letter “P” followed by the gene name according to the above described nomenclature, for example “PnirA” for the promoter controlling the transcription of the nirA gene. Truncated versions of the promoters including only a small portion of the native promoters upstream of the transcription start point, such as the region ranging from −35 to the transcription start can be used. Furthermore, introducing nucleotide changes in the untranslated region into the promoter sequence, e.g. into the TATA box, the operator sequence and/or the ribosomal binding site (RBS) can be used to tailor or optimize the promoter for a certain purpose. Such optimized promoters are identified by “*”, which may be followed by a number if more than one optimized version exists.

Denominations for enzyme names can be given in a two or three letter code indicating the origin of the enzyme, followed by the above mentioned three letter code for the enzyme itself, such as SynAdh (Zn²⁺ dependent alcohol dehydrogenase from Synechocystis PCC6803), ZmPdc (pyruvate decarboxylase from Zymomonas mobilis). Alternatively, the enzyme can be identified by the three letter code for the enzyme itself, optionally followed by a numerical identifier and/or a parenthesized reference to cases where the gene encoding the enzyme is a particular codon-optimized variant, e.g. PDC(AB1opt1) for a pyruvate decarboxylase variant or ADH111(AB1opt) for an alcohol dehydrogenase variant codon-optimized for expression in AB1.

The term “terminator” refers to a nucleic acid sequence, which is able to terminate the transcription of an mRNA. The terminators can exert their function in various ways including, but not limited to forming a hairpin structure in the mRNA transcript, which disrupts the mRNA-DNA RNA polymerase complex during transcription or via forming a recognition site for a transcription termination factor. Non-limiting examples are the dsrA terminator from E. coli, the oop terminator or the rho terminator.

All plasmid vectors used herein were synthetically produced by custom synthesis. It is well known to a person of ordinary skill in the art that large plasmids can be produced using techniques such as the ones described in the U.S. Pat. No. 6,472,184 B1 titled “Method for producing nucleic acid polymers” and U.S. Pat. No. 5,750,380 titled “DNA polymerase mediated synthesis of double stranded nucleic acid molecules”.

The plasmid vectors #1904 and #2042 are synthetic derivatives of an endogenous 6.8 kB extrachromosomal plasmid of Cyanobacterium sp. PTA-13311.

### Example 3

### Transformation of Cyanobacterium sp. PTA-13311

The Cyanobacterium sp. PTA-13311 has a significant layer of extracellular polymeric substances (EPS) outside the cell. The following method was used to decrease the EPS layer prior to conjugation. The method involves several steps: treatment of cells with N-acetylcysteine (NAC); washing steps that utilize NaCl; a treatment with lysozyme and subsequent washing. Firstly, 200 ml of an exponentially growing culture (0.5<OD₇₅₀ₙₘ<1) was incubated with N-acetylcysteine (NAC) for 2 days at 16° C. at 0.1 mg/ml final concentration without shaking. Afterwards, the culture was pelleted at 4400 rpm and washed with 0.9% NaCl containing 8 mM EDTA. The cell pellet was resuspended in 0.5 M sucrose and incubated for 60 minutes at room temperature (RT) with slow shaking at 85 rpm. Then, cells were centrifuged and resuspended in 40 ml of a solution containing 50 mM Tris pH 8.0, 10 mM EDTA pH 8.0, 4% sucrose, and 20-40 μg/ml lysozyme. After incubation at RT for 10-15 minutes, cells were centrifuged and washed three times using different washing solutions, namely i) with 30 mM Tris containing 4% sucrose and 1 mM EDTA, ii) with 100 mM Tris containing 2% sucrose and iii) with BG11 medium. All centrifugation steps before lysozyme treatment were performed at 4400 rpm for 10 min at 10° C., all centrifugations after the lysozyme treatment were performed at 2400 rpm for 5 minutes at 4° C.

Next, the cells were resuspended in 400 μl BG11 culture medium containing Tris/sucrose buffer and used for gene transfer via conjugation. Triparental mating was performed as follows. E. coli strain J53 bearing a conjugative RP4 plasmid and E. coli strain HB101 bearing the plasmid cargo to be introduced into Cyanobacterium sp. PTA-13311 and the pRL528 helper plasmid for in vivo methylation were used. E. coli strains were grown in LB broth supplemented with the appropriate antibiotics overnight at 37° C. with shaking at 100 rpm. An aliquot of 3-5 ml of each culture was centrifuged, washed twice with LB medium and resuspended in 200 μl LB medium. Subsequently, the E. coli strains were mixed, centrifuged and resuspended in 100 μl BG11 medium. A 100 μl aliquot of the resuspended cyanobacterial cells and the E. coli cultures was mixed and applied onto a membrane filter (Millipore GVWP, 0.22 μm pore size) placed on the surface of solid BG11 medium supplemented with 5% LB. Petri dishes were incubated under dim light of 5 μmol photons m⁻² s⁻¹ for two days. Cells were then resuspended in fresh BG11 medium and plated onto selective medium containing 10 and 15 μg/ml kanamycin, respectively. The following selection conditions were used: light intensity approximately 20-40 μmol photons m⁻² sec⁻¹ at a temperature of approximately 28° C. Successful transformants, also termed hybrids hereinafter, were visible after approximately 10-14 days. The transformant colonies were then plated on BG11 medium containing 15 μg/ml kanamycin and then stepwise transferred to higher kanamycin concentrations up to 60 μg/ml kanamycin to aid in the selection process.

### Example 4

### Generation of Ethanologenic orf0666 Deletion Strains and Reference Strains

The orf0666 gene encodes a homolog of the PhoU repressor in Cyanobacterium sp. PTA-13331. For knockout of the orf0666 gene, the Cyanobacterium sp. PTA-13311 host cells were transformed with an integrative suicide plasmid, which integrates into the genome of the host cell via homologous double-crossover recombination into the locus of an endogenous gene, leading to inactivation of this gene. In order to delete the orf0666 gene in Cyanobacterium sp. PTA-13311, the suicide plasmid #1980 was constructed. The construct harbors flanking regions about 3 kb in size homologous to the upstream and downstream region of orf0666. A codon-adapted version of the gentamycin resistance marker is placed in between the flanking regions driven by PcpcB and terminated by the bi-directional transcriptional terminator TB0014. The orf0666 deletion took place by homologous double-crossover integration into the corresponding regions of the orf0666 gene. Initially successful transformants, characterized by gentamycin resistance of the cells, were passed several times on BG11 medium supplemented with 2 mM ammonia and 2 mM urea and increasing gentamycin concentrations (10-200 μg/ml) until complete segregation of host cells with the orf0666 gene deletion was achieved.

Afterwards, ethanologenic strains AB0670 and AB0042 according to the present invention were generated by additionally transforming plasmid vectors #1904 (AB0670) or #2042 (AB0042) into the completely segregated Cyanobacterium sp. PTA-13311 delta orf0666 (deletion) strain.

As a reference, the ethanologenic strain AB0250 was generated by transforming the plasmid vector #1904 into a Cyanobacterium sp. PTA-13311 host strain without deletion of the orf0666 gene. Likewise, the reference strain AB0382 is capable of making ethanol and has an intact orf0666 gene.

### Example 5

### Determination of Cellular Uptake and Intracellular Storage of Inorganic Phosphate

The cellular phosphate uptake rate can be determined by time-dependent measurement of dissolved phosphate in the culture medium. The intracellular phosphate storage level can be determined by measuring the particulate phosphate in the cyanobacterial cell.

Phosphate can for example be determined with the BioVision “Phosphate Colorimetric Determination Assay Kit” (BioVision Inc., Milpitas, Calif., USA).

Preparation: 2 mL of cyanobacterial culture was centrifuged at 13,000×g for 10 min. 1.5 mL of the supernatant was transferred to a fresh 2 mL Eppendorf tube (SafeLock) and stored at −20° C. or used directly for measurement of dissolved phosphate in the medium, see “Phosphate measurement” below. The cell pellets were used for determination of the particulate phosphate (cellular phosphate), which additionally involved the following pre-treatment and digestion steps.

Pre-treatment: The cell pellets were washed with 2 mL phosphate-free growth medium. Afterwards, the cells were centrifuged at 13,000×g for 10 min and resuspended in 2 mL deionized water. The OD₇₅₀ₙₘ was adjusted with deionized water to about 0.2. Then, 1 mL of the OD-adjusted sample was transferred into a fresh SafeLock tube for the digestion process which converts particulate polyphosphate in orthophosphate.

Digestion: 80 μL persulfate reagent (500 mg potassium peroxodisulfate and 0.5 mL of 3 M sulfuric acid in 10 mL nano pure water) was added to the sample and digestion was maintained for at least 16 hours at 90° C. After digestion, the samples were cooled to room temperature and 120 μL of ascorbic acid (100 mM) was added directly to 1 mL of sample.

Phosphate measurement: 200 μL of the sample was transferred to a microwell plate and mixed thoroughly with 30 μL phosphate reagent provided in the BioVision kit. An incubation period of 20 min to max. 30 min at room temperature was maintained. Finally, absorption was measured at 650 nm. If the absorption exceeded the calibration range, the sample was diluted and retested.

The absorption values were compared to calibration curves. For particulate phosphate in cells, a calibration curve was prepared with 5 μM-60 μM phosphate standards in deionized water. The standards were taken through the digestion steps and the phosphate measurement was carried out as described above. For determination of dissolved phosphate in medium, standards of phosphate from 5 μM-60 μM were prepared in the appropriate medium and the phosphate measurement was carried out as described above.

### Example 6

### Phosphate Uptake and Storage of Strains AB0670 and AB0250 Under Different Conditions

Strain AB0670 according to the present invention and reference strain AB0250 were scaled-up in ASW-(artificial seawater)-based marine BG11 (mBG11) medium with 4 mM urea and 5 mM TES pH 7.3. The pre-cultures were used to inoculate main cultures in 0.5 L bottles with phosphate-free mBG11 medium at an OD₇₅₀ₙₘ of 0.5. Cultivation was maintained at 350 and 1% CO₂ with a flow rate of 200 mL/min. Phosphate was added to the main cultures in a concentration of 1,000 μM phosphate per OD₇₅₀ₙₘ from a 230 mM K₂HPO₄ stock solution. One set of cultures was fed directly after inoculation, the second set after one day of phosphate starvation and the third set after two days of starvation in order to obtain different phosphate pre-loading levels in the cells.

On day three, all cultures were fed again with 1,000 μM phosphate per OD₇₅₀ₙₘ to investigate the phosphate uptake for the different phosphate pre-loaded states of the cells. For the phosphate uptake measurements, 1.5 mL samples were taken from the cultures 0, 15, 30, 45 and 60 min after the addition of the phosphate on day three. The samples were centrifuged at 4° C. for 10 min with 13,000×g in a benchtop centrifuge. 1 mL of the supernatant was used for determination of dissolved phosphate as described in Example 5 above. The phosphate uptake rate was then determined by linear regression analysis of the time-dependent phosphate depletion in the medium.

For determination of the intracellular phosphate storage ability of the strains, 2 mL of the cultures were taken immediately before the phosphate addition on day three and 24 h later. The intracellular phosphate levels were again determined as described in Example 5 above.

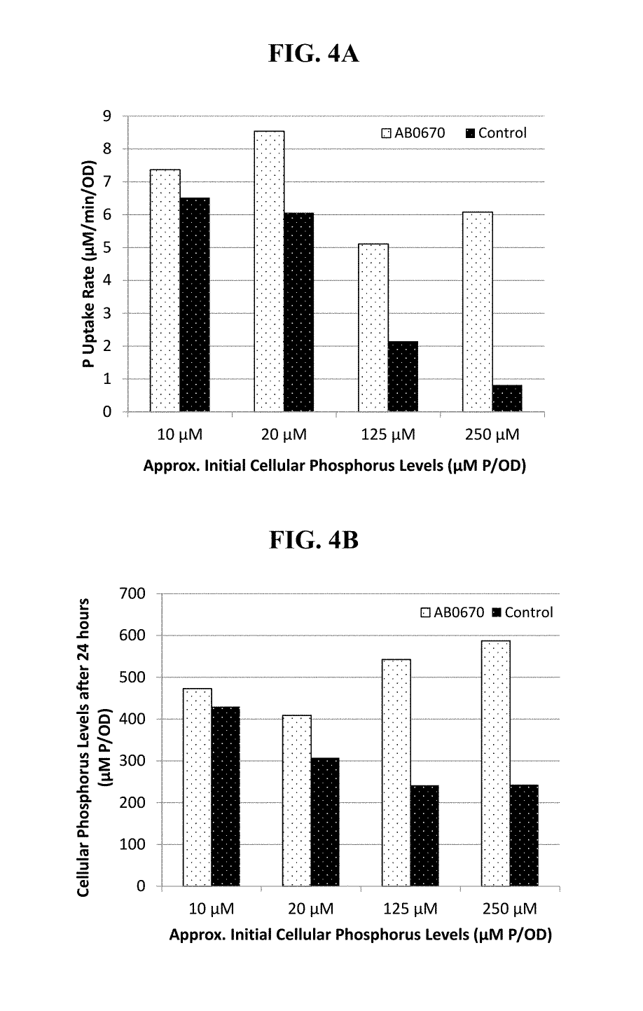

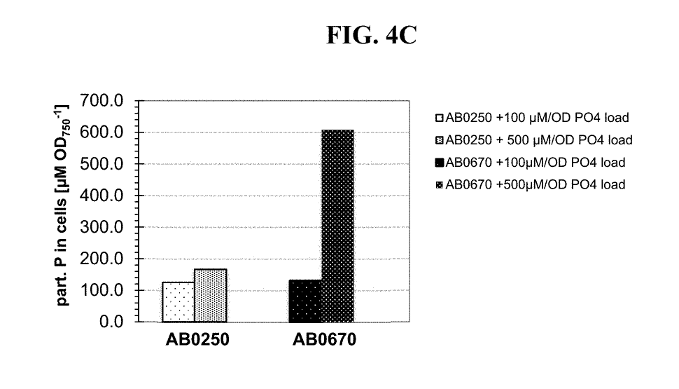

FIG. 4A shows the phosphate uptake rate (in μM per min and OD₇₅₀ₙₘ) of strain AB0670 of the present invention and reference strain AB0250, termed “Control”, in relation to the initial intracellular phosphate level in the culture medium. The results show that the strain AB0670 generally took up phosphate faster than the reference strain. The highest uptake rates of about 7.3 and 8.5 μM per min and OD₇₅₀ₙₘ were observed when the lower intracellular levels of 20 μM and 10 μM phosphate were present. Strikingly, however, AB0670 continued to take up phosphate from the medium even at high intracellular phosphate levels of 125 μM and 250 with only slightly decreased uptake rates of about 5 and 6 μM per min and OD₇₅₀ₙₘ. The difference in the phosphate uptake rate between AB0670 and AB0250 was most prominent at the high intracellular phosphate levels, where a more than 6-fold faster uptake was observed.

FIG. 4B shows the corresponding cellular level of phosphate in μM per OD₇₅₀ₙₘ of strains AB0670 and AB0250 after 24 hours of cultivation. The results show that the strain AB0670 generally stored more phosphate than the reference strain. While the reference strain tended to accumulate more intracellular phosphate under phosphate starvation conditions and less intracellular phosphate when high medium concentrations of phosphate were present, strain AB0670 exhibited essentially consistent high phosphate storage levels between about 470 and 690 μM phosphate per OD₇₅₀ₙₘ. This property is particularly beneficial with regard to an efficient and reliable phosphate loading of the cyanobacterial cell during scale-up of the culture.

FIG. 4C shows the results of intracellular phosphate storage of strain AB0670 and reference strain AB0250. The strains were scaled-up in mBG11⁰ medium pH 8 supplemented with 4 mM urea, 5 mM TES, 0.064 μM CuSO₄ until an OD₇₅₀ₙₘ of 3-4 was reached. Afterwards, the cells were loaded with phosphate by adding either 100 μM phosphate per OD₇₅₀ₙₘ or 500 phosphate per OD₇₅₀ₙₘ to the medium and maintaining cultivation for another 24 hours. Under the high phosphate-loading condition with 500 μM phosphate per OD₇₅₀ₙₘ, strain AB0670 exhibited an about 3.75-fold higher phosphate loading capacity than the reference strain. Notably, the medium of the AB0670 culture did not contain any detectable amount of phosphate after the loading, which shows that the strain was able to take up the available phosphate completely. In this way, carryover of residual phosphate which could trigger contaminant growth during the production phase is effectively avoided. Conversely, significant concentrations of phosphate remained in the medium of the AB0250 culture.

In conclusion, the strain AB0670 according to the present invention is characterized by a faster and more efficient cellular phosphate uptake and higher intracellular phosphate storage compared to the reference strain AB0250.

### Example 7

### Ethanol Production and Contaminant Growth in Cultures of Strains AB0670 and AB0250

Pre-cultures of recombinant strain AB0670 and reference strain AB0250 were scaled-up and loaded with 500 μM phosphate per OD₇₅₀ₙₘ as described in Example 6 above. Afterwards, each strain was used to inoculate a main culture of 0.5 L in ASW-BG11 medium supplemented with 5 mM TES pH 7.3 and 4 mM urea but lacking phosphorus. The inoculation density corresponded to an OD₇₅₀ₙₘ of 0.2. CO₂ was supplied to the cultures by bubbling with 1% CO₂ in a flow rate of 50 mL per minute. The cultures were illuminated with 350 μE from one side for a period of 12 hours per day.

The main cultures were spiked with Alcanivorax as a representative example of a heterotrophic, ethanol consuming contaminant. Before spiking, Alcanivorax strain 76_12 was cultured for five days in ASW-BG11 medium lacking phosphate supplemented with 0.5 vol % ethanol. The main cultures were spiked with 100 CFU Alcanivorax 76_12 per mL at the start of the cultivation. On day 15, the cultures were fed with phosphate in the form of 0.1 M K-PBS buffer pH 7.0 (61.5% of 0.1 M K₂HPO₄ and 38.5% of 0.1 M KH₂PO₄) at a concentration of 30 μM per OD₇₅₀ₙₘ.

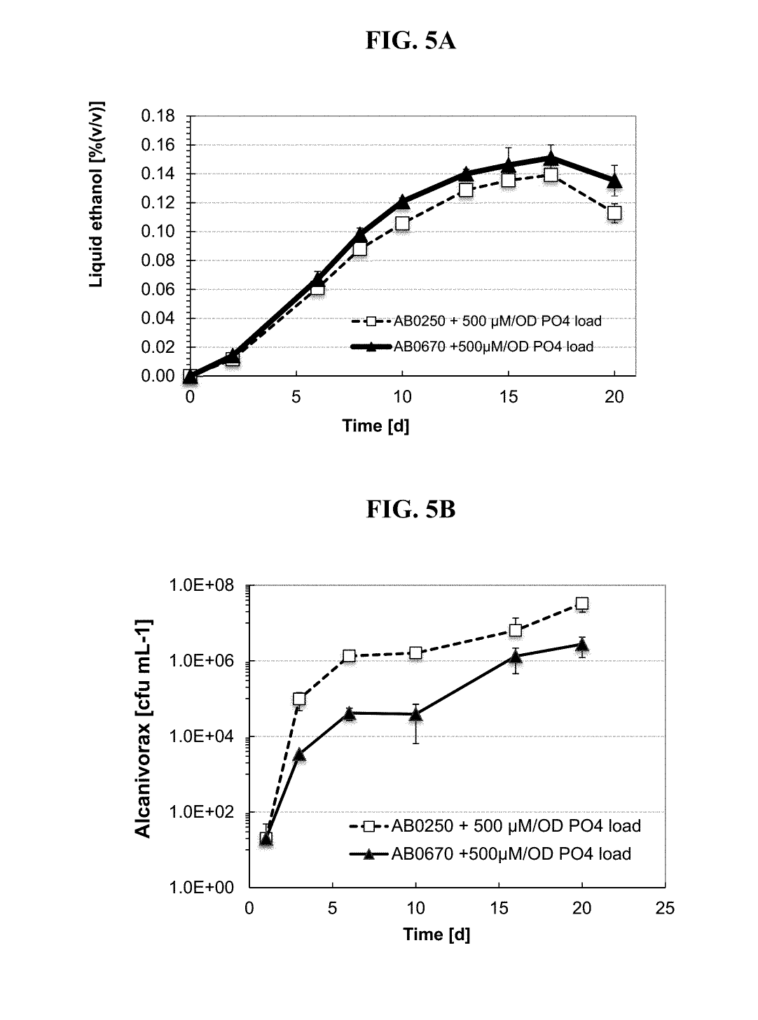

FIG. 5A shows the ethanol production with the AB0670 culture and the AB0250 culture over a period of 20 days. Ethanol content in the medium was measured by gas chromatography. For this purpose, 0.5 mL of the culture was transferred into a 20 mL GC vial and ethanol content of the sample was measured in the gas phase of the vial after heating the sample to 45° C. with shaking. Ethanol content was determined by comparison of the GC results with a calibration curve prepared from standards with predetermined ethanol concentrations in medium. Data points represent arithmetic means and standard deviations of two independent samples. More ethanol was present in the AB0670 culture than in the AB0250 reference culture at any time during the cultivation.

FIG. 5B shows the concomitant growth of Alcanivorax in CFU per mL in the cultures over the monitored 20 days. Between day 3 and day 20 of the cultivation, the abundance of Alcanivorax was on average reduced by about 90% in the AB0670 cultures in comparison to the AB0250 cultures.

In conclusion, an improved contamination control and improved ethanol yield is achieved with strain AB0670 of the present invention in comparison to the reference strain AB0250.

### Example 8

### Ethanol Production and Contaminant Growth in Outdoor Cultivation of Strains AB0042 and AB0382

The ethanologenic phoU (orf0666) knock-out strain AB0042 according to the present invention and the ethanologenic reference strain AB0382 without deletion of orf0666 were cultured in outdoor photobioreactors. The reactors were operated semi-continuously in three batches of about 23 cultivation days per batch. The second batch was inoculated directly with a 4.5-fold dilution of the first batch, the third batch was inoculated directly with a 8.0-fold dilution of the second batch. A single clean-in-place treatment (CIP) was conducted prior to the first batch of AB0042 and AB0382. An additional control cultivation of reference strain AB0382 however included subsequent CIP treatments between the batches, wherein each batch was freshly inoculated.

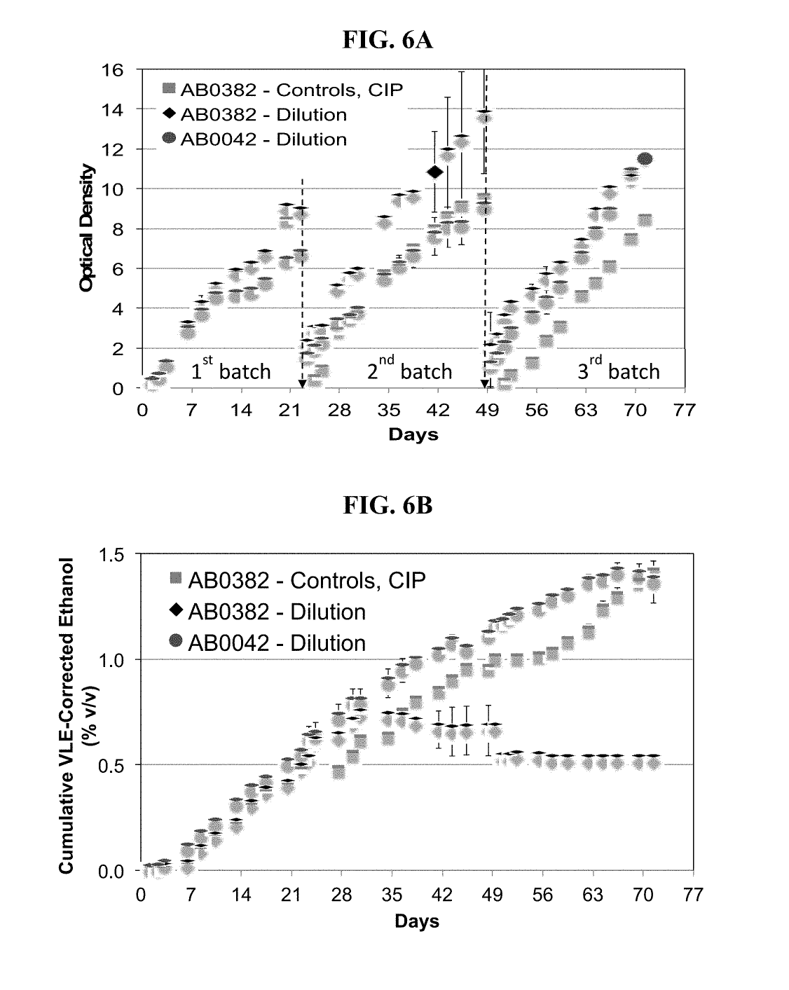

FIG. 6A shows the cyanobacterial growth in the reactors during the three batches as optical density at 750 nm over cultivation days. All strains showed essentially consistent growth in all three batches. The biomass produced by AB0042 was slightly lower than that produced by AB0382 during the first and second batch.

FIG. 6B shows that AB0042 exhibited a consistent ethanol production through 65 days from a single CIP (“AB0042-Dilution”). In contrast, the production with AB0382 from a single CIP (“AB0382-Dilution”) lost ethanol to contamination as of the middle of the second batch and throughout the third batch. Ethanol loss in the culture of reference strain AB0382 was only avoided when contaminant growth was reduced by additional CIP treatments in between batches (“AB0382-Controls, CIP”). In total, about 12% more ethanol was produced with AB0042 compared to the AB0382 control with additional CIP treatments through 63 days and over 100% more compared to the AB0382 cultivation without additional CIP.

These results demonstrate that a continuous ethanol production is possible with the delta orf0666 recombinant cyanobacterium and the complementary cultivation methods according to the present invention, since contaminant growth and associated product loss is efficiently reduced. In addition to an improved ethanol yield, the economic efficiency of the cyanobacterial ethanol production is increased since less reactor downtime and labor is needed for CIP treatments.

### Example 9

### Preparation of Integrative Suicide Plasmids for Transforming Other Cyanobacterial Strains with the phoU Knock-Out (KO) Construct

In a prophetic example, a cyanobacterial strain of interest is chosen for the production of a compound of commercial interest. Next, a PhoU homolog is identified in the cyanobacterial strain. The DNA sequence of the phoU homolog gene is determined. The DNA sequence of the 2K upstream and 2K downstream regions is also determined. An integrative suicide plasmid is prepared from the sequence information. In this instance, the term “suicide plasmid” refers to a plasmid with no ability to replicate in the targeted cyanobacterial host cell. A host cell of the strain of interest is transformed with the suicide plasmid, which integrates into the locus of an endogenous gene of the host cell genome via homologous double-crossover recombination, leading to inactivation of the phoU gene. An antibiotic resistance gene is placed in between the flanking regions, and is driven by a chosen promoter and terminated by a bi-directional transcriptional terminator. Successful transformants containing the phoU knock-out are characterized by resistance to the antibiotic, and are passed several times on a suitable cyanobacterial growth medium supplemented with the antibiotic until complete segregation of the host cell with the phoU knock-out deletion is achieved (that is, no copies of the native phoU gene remain). The strain is then tested multiple times to ensure that the mutation is present in all of the chromosomal copies. Additional genetic manipulation is performed as needed so that the cell is able to produce a compound of interest.

By use of this method, the modified strain can take up phosphorus in the medium at a much faster rate when grown over an extended range of external phosphorus levels in the medium than a similar but unmodified strain. The modified strain can also store more phosphorus in the cell as a polyphosphate moiety. Further, the new modified strain can more easily out-compete non-photosynthetic bacteria or other organisms for the available phosphorus in the medium. Using this method, a compound of interest can be produced at higher levels, with less contamination of the cyanobacterial culture, than would be present in a control cell culture not having the phoU gene deletion.

### Example 10

### Knock-Out of phoU Gene in Synechococcus PCC 7002

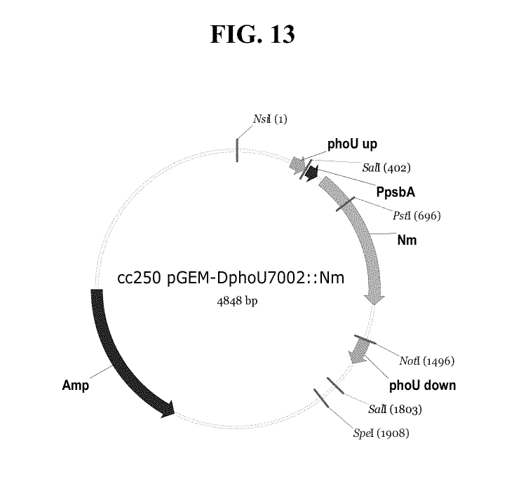

The gene encoding the PhoU homolog from Synechococcus PCC 7002 was identified (SEQ ID NO: 44). A suicide plasmid construct was designed having the gene sequence, along with a 5′ and 3′ flanking region, as well as a neomycin/kanamycin resistance gene. A plasmid map is shown in FIG. 13. The sequence for the complete plasmid is SEQ ID NO: 45. A plasmid having this sequence is transformed to a Synechococcus PCC 7002 host cell. Transformation is confirmed, and increasing amounts of kanamycin are added through several passes to result in a host cell that no longer contains the active, native phoU gene. Once the knock-out of the phoU gene is confirmed, the strain is scaled-up and the culture is tested for its ability to take up P at a much faster rate than a similar host cell that does not have the phoU knock out.

By use of this method, Synechococcus PCC 7002 cells are capable of faster uptake of available P in the medium.

### Example 11

### Knock-Out of phoU Gene in Anabaena variabilis ATCC 29413

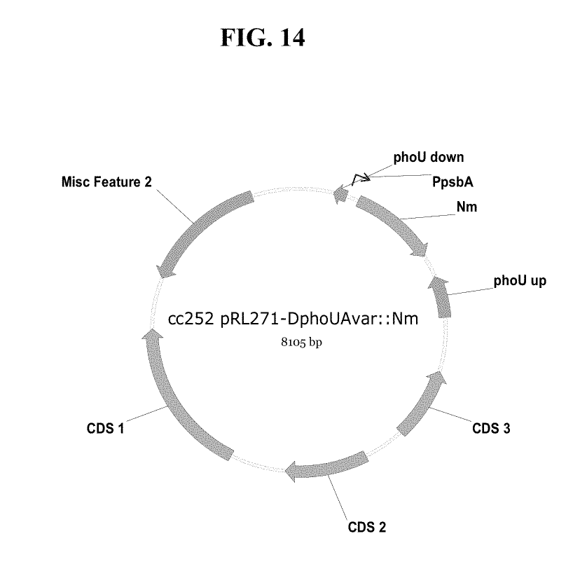

The gene encoding the PhoU homolog from Anabaena variabilis ATCC 29413 was identified (SEQ ID NO: 46). A suicide plasmid construct was designed having the gene, along with a 5′ and 3′ flanking region, as well as a neomycin resistance gene. A plasmid map is shown in FIG. 14. The sequence for the complete plasmid is SEQ ID NO: 47. A plasmid having this sequence is cloned in E. coli and transformed to an Anabaena variabilis ATCC 29413 host cell. Transformation is confirmed, and increasing amounts of neomycin are added through several passes to result in a host cell that no longer contains the native phoU gene. Once the absence of the phoU gene is confirmed, the strain is scaled-up and the culture is tested for its ability to take up P at a much faster rate than a similar host cell that does not have the phoU knock out.

By use of this method, Anabaena variabilis ATCC 29413 cells are capable of faster uptake of available P in the medium, as well as the ability to store more P inside of the cell. The modified cells are better able to compete for available P in the culture medium. Less contamination of the culture occurs.

### Example 12

### Pre-Loading the Cyanobacterial Cells with Phosphoric Acid as the P Source

To confirm that cyanobacterial cells containing the phoU knock-out can be pre-loaded with even higher amounts of phosphorus using phosphoric acid, a 7 liter vertical photobioreactor was filled with sterilized artificial seawater (“ASW”)-based marine BG11 (mBG11) medium (nitrate free and phosphate-free) with 5 mM urea. The culture medium was loaded with high amounts of phosphorus in the form of phosphoric acid in varying amounts to duplicate photobioreactors:

1) 600 μM, added at one timepoint (1×), (final load of 150 μM/OD₇₅₀ₙₘ)

2) 1,200 μM, added at one timepoint (1×), (final load of 350 μM/OD₇₅₀ₙₘ)

3) 900 μM, added at two timepoints (2×), (final load of >500 μM/OD₇₅₀ₙₘ)

The final cellular P load was determined based on a final OD of −3.0 at the time of harvest. The pH was adjusted manually. The cells grew to an OD₇₅₀ₙₘ of about 3.0 in 2-3 days. In some samples the pH briefly went below 7.0 (the dip ranged from about pH 5.5-7.0), at the time of phosphoric acid addition but readjusted to a pH of about 8.0 by the next day. It was necessary to add phosphoric acid by multiple additions when adding greater than 1200 μM total to the culture so that the pH would remain in a suitable range for cell survival.

The cells took up and stored all of the phosphorus by 48 hours. Notably, even the culture with a very high load (2×900 μM) was able to take up all of the P and showed very high levels of phosphorus inside the cell at the end of the run, averaging about 500 μM/OD₇₅₀ₙₘ. These cells were then used as inoculum for outdoor production photobioreactors.

The results demonstrated that very large amounts of P can be taken up and stored in the cyanobacterial cells when it is provided to the cells in the form of phosphoric acid, with no deleterious effect on cell growth or ethanol production during the ethanol production phase after the phosphorus pre-load. Additionally, unlike the commonly used potassium phosphate, phosphoric acid does not form a white precipitate when it is administered to the culture at high levels. Phosphoric acid is also less expensive than other commonly used phosphorus sources, such as potassium phosphate. Further, the feedstocks can better resist biofouling because of the lower pH.

### Example 13

### Competition Dynamics of P-loaded cells

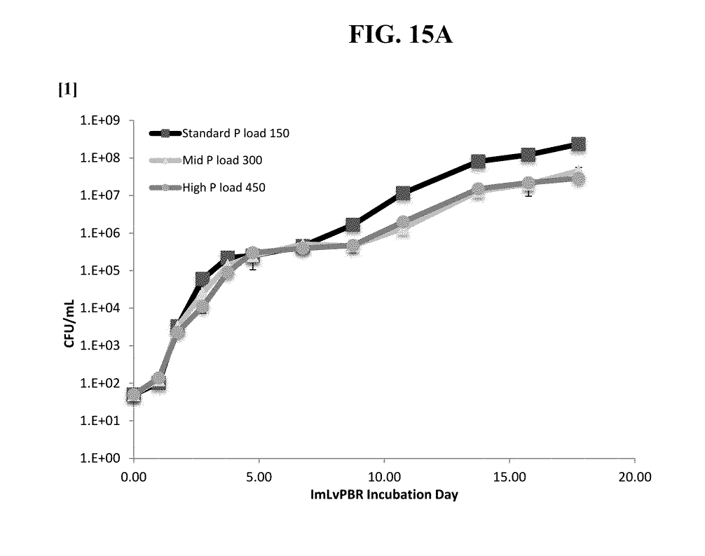

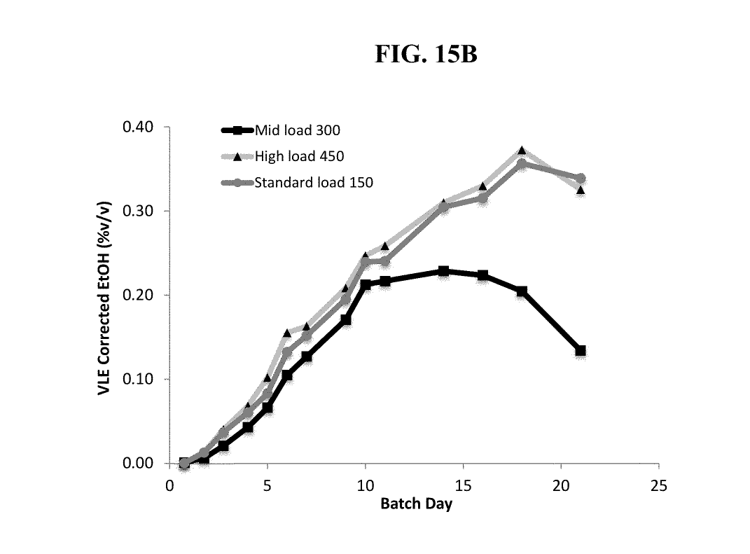

To better understand the competition dynamics with other organisms once the P-loaded cells are inoculated to the final production culture, a sample of the above cells (strain AB0042) was inoculated into 350 ml indoor photobioreactors with 1×BG-11 (P-free), with delayed induction (of ethanologenesis) and periodic additions of 35 μM phosphoric acid based on cellular need. The culture was grown with 350 μE light with a photoperiod of 12 hours on/12 hours off. To simulate outdoor contamination, a purified aliquot of the contaminant bacterium Alcanivorax sp. was added at a starting density of 50 cells/ml. The CO₂ supply was ramped from 0.25% to 5%, over the course of 28 days. The cells were grown for four weeks. After the four-week growth period, the added Alcanivorax population was enumerated and ethanol levels were measured. The higher P-loaded cultures had about 50% less Alcanivorax cells/ml, and had a delay in ethanol consumption by the Alcanivorax test organism. (FIG. 15A).

Cyanobacterial cells having a disruption of the phosphate uptake regulation gene phoU were capable of storing a large amount of phosphorus in the cell in the form of phosphate storage bodies. Thus, this method can be utilized to grow cyanobacterial cells with less contamination than reference control cells, because when the cultures are in the final production phase (and more likely to become contaminated), the cyanobacterial cells have already been pre-loaded with high amounts of P. The cells were able to grow for a longer period of time during the production phase without the need to add additional P, and were also able to outcompete the contaminants for the very limited amount of phosphorus that may be present in the surrounding medium. The contaminants, in contrast, had limited access to P until the first P feeding was performed. The delay in the need for P feeding that is due to the high pre-load of P can keep the ethanologenic culture performing satisfactorily for a longer period. In this experiment, the ethanol accumulation for the first 2 weeks of the culture that had been previously loaded with a large amount of P was similar to axenic runs of similar strains (FIG. 15B).

When the product is a compound (such as ethanol) that can be consumed by contaminating bacteria, the use of this method allows for lower bacterial contamination (or a delay in bacterial contamination) and lower consumption of the compound, resulting in a higher accumulation of the compound of interest at the end of the run (as shown in FIG. 15B).

### Example 14

### Pre-Loading with Large Amounts of P (as Phosphoric Acid) Allows for Longer Term Culture Growth and Product Production

Method: An ethanologenic, phoU knockout strain of cyanobacteria was cultured in a 7 L outdoor vertical photobioreactor to pre-load enough P (as phosphoric acid) so that the culture would be able to run for a 30 day batch without an additional phosphorus feed. The scale-up photobioreactors were inoculated at an OD₇₅₀ₙₘ of 0.2 using culture medium having BG-11 nutrients (nitrate free and phosphate free, amended with 5 mM urea; termed “BG-11⁰”). Phosphoric acid was added to the culture for a targeted final load of 1,500 μM P/OD₇₅₀ₙₘ. This was achieved by addition of 4,500 μM phosphoric acid over the course of 2 days using slow addition, adding increasing levels of phosphoric acid every 1-2 hrs during the photoperiod. After three days, a high P load of 1,500 μM P/OD₇₅₀ₙₘ was obtained.

The cells were then inoculated at an OD₇₅₀ₙₘ of 0.2 to indoor 350 mL vertical photobioreactors (BG-11 nutrients, phosphate free, amended with 0.75 mM urea) to determine whether the culture could grow for 30 days without the need for additional P. A control photobioreactor was cultured with a similar ethanologenic cyanobacterial cell strain, except that it did not have the phoU knock-out. The control culture was fed with 35 μM P (as phosphoric acid) periodically (every few days, as noted by the stars at the bottom of the graph) under standard cultivation conditions.

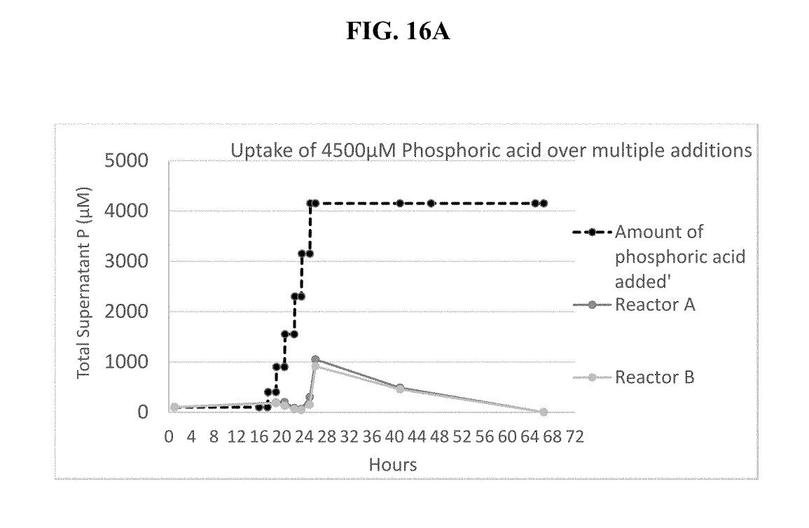

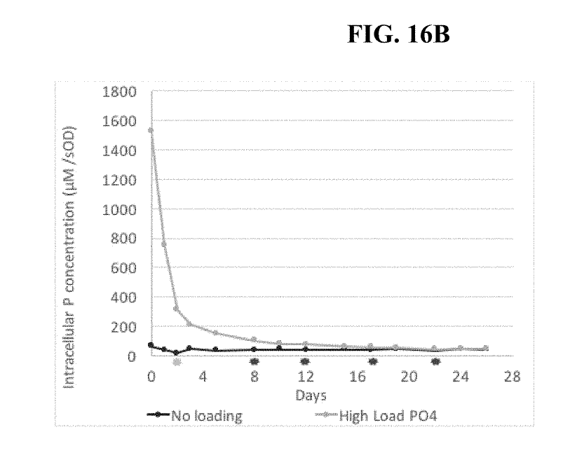

Results: (See FIG. 16A and FIG. 16B). The phosphorus loaded phoU knock-out culture had similar cell growth as the control strain, and produced an equivalent amount of ethanol as the control culture. The phoU knock-out culture also had enough stored intracellular phosphorus to last the entire 30 day batch run without needing additional phosphorus feeding. Thus, the phosphorus was successfully loaded into the PhoU knock-out cell during the scale-up phase, and the culture could then be grown without phosphorus for the outdoor, production phase. This is useful for the production phase of the compound in outdoor photobioreactors, because the photobioreactors are less likely to become contaminated when phosphorus is not present in the culture medium. The phoU knockout strain was capable of being loaded with a much higher amount of phosphorus per cell than a similar strain that does not have the phoU knockout.

FIG. 16A shows the phosphoric acid uptake rate, as well as the total amount of phosphorus supplied to the culture. The total amount of phosphorus added, as well as the amount of P remaining in the medium, was measured over time. This graph also shows that phosphorus (as phosphoric acid) can be taken into the cell very quickly, and to very high levels.

The intracellular storage of phosphorus during the phosphorus-preloading phase was also measured (FIG. 16B). The black line is the standard control condition, which had to be fed five times with phosphoric acid (indicated by star) over the course of 30 days.

### Example 15

### Production of Isoprene

The cyanobacterial strain Cyanobacterium sp. PTA-13331 is transformed with a construct to knock out its endogenous phoU gene, essentially following the method described in Example 4. Once the transformation is complete and the strain is fully segregated for the phoU knock-out, the strain is further modified by the transformation with a plasmid containing a gene encoding isoprene synthase (“ispS”: EC 4.2.3.27), essentially following the methods described in U.S. Patent Application Publication No. 20150232884, which is incorporated by reference herein in its entirety. The strain is scaled up under axenic conditions, with phosphoric acid pre-loading to approximately 500 μM P per OD₇₅₀. The culture is then inoculated to outdoor bioreactors filled with mBG11 medium without phosphate, and the culture remains for at least 45 days without needing additional phosphorus additions. After 45 days, isoprene is harvested from the culture medium.

By use of this method, there is less contamination of the culture, and the modified cyanobacterial cells can grow and produce isoprene for a longer period of time in an outdoor production environment, with less detrimental effects from the presence of contaminating organisms.

### Example 16

### Production of Biomass

Synechococcus PCC 7002 is transformed with a construct to knock out its phoU homolog gene, essentially following Example 9 and Example 10. Once the transformation is complete and the strain is fully segregated for the phoU knock-out, the strain is scaled up under axenic conditions with an elevated amount of phosphate (750 μM P per OD₇₅₀), in order to pre-load the culture with phosphate while it is still under axenic conditions. The culture is then inoculated to outdoor photobioreactors filled with BG11 medium without phosphate. The culture grows for 2 weeks; then biomass is harvested, leaving 5% of the culture in the photobioreactors. The photobioreactors are re-filled with BG11 medium without phosphate, and biomass is again harvested after 2 weeks. A small amount of P can be added as needed at intervals throughout the culture run. The modified cyanobacterial cells are able to take up the added P faster than the contaminants can. This process of growth and harvest is continued until the culture becomes substantially contaminated.

By use of this method, the modified cells can take up larger amounts of P while in an axenic scale-up phase, and can then grow well in the production phase in the presence of minimal P in the medium. Fewer contaminating organisms can grow in the P-limited medium, while the modified cyanobacterial culture can continue growing and producing biomass for a longer amount of time. This results in lower production costs for biomass production.

Example embodiments have been described herein. As may be noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments, but should be defined only in accordance with features and claims supported by the present disclosure and their equivalents. Moreover, embodiments of the subject disclosure may include formulations, compounds, methods, systems, and devices which may further include any and all elements/features from any other disclosed formulations, compounds, methods, systems, and devices, including the manufacture and use thereof. In other words, features from one and/or another disclosed embodiment may be interchangeable with features from other disclosed embodiments, which, in turn, correspond to yet other embodiments. One or more features/elements of disclosed embodiments may be removed and still result in patentable subject matter (and thus, resulting in yet more embodiments of the subject disclosure). Furthermore, some embodiments of the present disclosure may be distinguishable from the prior art by specifically lacking one and/or another feature, functionality, ingredient or structure, which is included in the prior art (i.e., claims directed to such embodiments may include “negative limitations” or “negative provisos”).

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A recombinant cyanobacterial cell for the production of a chemical compound of interest, the recombinant cyanobacterial cell comprising:
a) at least one genetic modification that alters expression of at least one endogenous phosphate uptake regulating gene encoding a protein involved in regulation of phosphate metabolism, wherein cellular uptake and/or intracellular storage of a phosphate compound is increased in comparison to a native form of the cyanobacterial cell; and
b) at least one recombinant production gene encoding an enzyme for the production of the chemical compound of interest;
wherein the protein involved in regulation of phosphate metabolism regulates expression and/or activity of at least one phosphate transport protein involved in transfer of the phosphate compound from an external solution into the cyanobacterial cell.

2. The recombinant cyanobacterial cell of claim 1, wherein the protein involved in regulation of phosphate metabolism represses expression and/or activity of the at least one phosphate transport protein in the cyanobacterial cell in its native form.

3. The recombinant cyanobacterial cell of claim 1, wherein the genetic modification causes constitutive expression and/or activity of the at least one phosphate transport protein.

4. The recombinant cyanobacterial cell of claim 1, further comprising a phosphate transport complex that comprises the at least one phosphate transport protein and additional proteins.

5. The recombinant cyanobacterial cell of claim 1, wherein the at least one phosphate transport protein and/or additional protein is selected from the group consisting of PstS, PstC, PstA, PstB, and combinations thereof.

6. The recombinant cyanobacterial cell of claim 1, wherein the at least one phosphate transport protein comprises a phosphate-binding protein capable of binding the phosphate compound.

7. The recombinant cyanobacterial cell of claim 1, wherein the at least one phosphate transport protein comprises an amino acid sequence having at least 16 identical residues without gaps to a consensus sequence VNYQSVGSGAGLRQFIXGTVDFAGSDLPL (SEQ ID NO: 41), wherein X is any one of the 20 natural amino acids.

8. The recombinant cyanobacterial cell of claim 1, wherein the genetic modification comprises a heterologous nucleic acid sequence encoding a knockdown component that reduces or eliminates the expression of the phosphate transport-regulating protein, wherein the knockdown component comprises RNA transcribed from the heterologous nucleic acid sequence that is at least partially complementary to mRNA transcribed from the phosphate uptake regulating gene.

9. The recombinant cyanobacterial cell of claim 1, wherein the genetic modification comprises at least partial disruption or complete removal of the phosphate uptake regulating gene.

10. The recombinant cyanobacterial cell of claim 1, wherein the protein involved in regulation of phosphate metabolism contains a full length match with at least 8 out of 13 identities to an amino acid sequence DLERIGDLAXNIA (SEQ ID NO: 42), wherein X is any one of the 20 natural amino acid, and/or a full length match with at least 9 out of 13 identities to an amino acid sequence LERIGDHATNIAE (SEQ ID NO: 43).

11. The recombinant cyanobacterial cell of claim 1, wherein the phosphate compound comprises an inorganic phosphate compound comprising an orthophosphate selected from the group consisting of H₂PO₄⁻, HPO₄²⁻ and/or PO₄³⁻.

12. The recombinant cyanobacterial cell of claim 1, wherein the enzyme for the production of the chemical compound of interest comprises a pyruvate decarboxylase, further wherein the chemical compound of interest is ethanol.

13. A method for producing a chemical compound of interest with a recombinant cyanobacterial cell, comprising:
(A) culturing the recombinant cyanobacterial cell of claim 1 in a phosphate compound-poor medium containing from about 0 μM to about 100 μM of the phosphate compound, the cyanobacterial cell expressing the enzyme, thereby producing the chemical compound of interest.

14. The method of claim 13, wherein step (A) comprises a non-axenic culturing condition with heterotrophic microorganisms present, and during step (A), the loss of the chemical compound of interest caused by the heterotrophic microorganisms is maintained below about 20% of the total amount of the chemical compound of interest produced.

15. The method of claim 14, further comprising, prior to step (A),
(A′) culturing the cyanobacterial cell in a phosphate compound-rich medium containing more than about 100 μM of the phosphate compound, the cyanobacterial cell taking up the phosphate compound.

16. The method of claim 15, wherein the phosphate compound-rich medium in step (A′) and the phosphate compound-poor medium in step (A) are provided as separate media.

17. The method of claim 15, wherein step (A′) comprises depletion of the phosphate compound from the phosphate compound-rich medium in an amount of time from about 1 to about 72 hours by the recombinant cyanobacterial cell, thereby resulting in the phosphate compound-poor medium, and step (A) comprises continuing culturing the recombinant cyanobacterial cell in the phosphate compound-poor medium of step (A′) resulting from the depletion.

18. The method of claim 13, wherein step (A) further comprises at least one addition of the phosphate compound to the phosphate compound-poor medium during culturing of the cyanobacterial cell, in an amount from about 1 μM to about 100 μM of the phosphate compound based on the final concentration of the phosphate compound in the cyanobacterial culture.

19. A method for reducing or preventing loss of a chemical compound of interest produced in cyanobacteria due to consumption by heterotrophic microorganisms, comprising growing a cell of claim 1 in a culture to produce the compound of interest, wherein the growth of the heterotrophic microorganisms is reduced or inhibited by limiting availability of the phosphate compound in the cyanobacterial culture.

20. A method for producing the cyanobacterial cell of claim 1, comprising:
a) providing at least one transformable nucleic acid construct for the genetic modification that alters expression of the at least one phosphate uptake regulating gene encoding a protein involved in regulation of phosphate metabolism and at least one transformable nucleic acid construct comprising the at least one recombinant production gene encoding an enzyme for the production of the chemical compound of interest; and
b) transforming the transformable nucleic acid constructs into a cyanobacterial cell;
wherein the transformed cyanobacterial cell has a higher rate of phosphate uptake and/or storage than an otherwise identical cyanobacterial cell that does not have the genetic modification that alters expression of the at least one phosphate uptake regulating gene.


SEQUENCE LISTING (47 sequences)

SEQ ID NO: 1 - DNA, 9707 bp, Artificial sequence
  Synthetic plasmid sequence with -orf666 (PhoU) deletion cassette
aaaaagttgtcaaaaaattaagtttctttacaaatgcttaacaaaaacttggttttaagcacaaaataagagagactaatttgcagaagttttacaaggaaatcttgaagaaaaagatctaagtaaaacgactctgtttaaccaaaatttaacaaatttaacaaaacaaactaaatctattaggagattaactaaaaccatggtaagatcaagtaacgacgtaacccagcaaggtagtagacctaaaacaaagttgggaggctcaagtatgggtatcattcgcacttgtaggttagggcccgatcaagtgaaatctatgcgagctgctttagatctttttggtagagaatttggtgatgttgcaacttatagtcaacatcaaccagattccgactatttgggaaatcttttacgttctaaaacattcattgcattagcagcatttgatcaggaagctgttgtcggtgcattagccgcttatgttttacccaagtttgaacaacctcgttctgagatctatatctacgatttagcggtatctggcgagcatcgtcgccaagggatagcaacagcgttaatcaatttgttaaagcatgaagctaatgctcttggtgcctatgtgatttacgttcaagcggactatggagatgatcctgccgtagccttgtataccaaacttggaattagagaagaagttatgcactttgatattgatccttctactgctacttaacaattcgttcaagccgagtcacactggctcaccttcggtgggcctttctgcgtttatatactagagagagaatataaaaagccagattattaatccggcttttttattatttgaagttcctattctctagaaagtataggaacttcatttaaattacgtacacggcggccgctgcctaccttcaccgacaaatttatatcgaactcaactaaatcaaacattcaggctgaacaacagaggtttatttgacccaaacctgagctttggataagctgaaagtattattttctcctagtcagaaaaccttatagcttcttagaaataacgataaaattgccttaatccgaactgacgttaaatatattcaacccctacttccccctctcaccctctcccctcatctcctcatcaccctaacacctacaacctgacacctgacatctaaccttatcggatattcttaaaccgaactgaggttgacccaaagacctgtaaatgataatatagcctagacataaaaccttgatcgccccttggaatatagataattatgggaaaaatttgggagttagacttttattctcgtccaattattgatgaaaataacaaaaaacgttgggaaatcctcatctgtgaaagtcctaccactatagacaccgatacaagccaattatttcgctattctcaattttgcgccaatacagaagttaactccattactcttcaaaatgcgatcgcaactgccatagaaaaagcaggagaaactcccagtaaaatccgcttctttcgtcgtcaaatgaataacatgattcttaaaggatgtgaagatgcggggattccagcccttgcatcccgtcatacttatactctcaatcaatggttagaagagagaatgacatcattttatcccctccaagaaggctatgacgaaaaaaccaccattgccgcttctgttcaatatcctcaaaccaacccagtaaatcttcctgatgctttaaaaggagataaaaaagacaaatgggcattagttagtttaaatgggaaagatttagaagaaatgcccgaatgggatataggttttagagaggcgtttcccctaaaaatagccaatatatctccagacaccaaaattccggggctaattatcttttcctcccgtgctttacctcttgcaggatggatgtctggactggaattaggttacttaagattagatagaggaaagttcccctgtatttgcttagaaacaggggtgagcgatagttggatattagttaacttgacggacaaaaacaccttatctgaggcagaaggctttgaaaatacgaaaaaacaggctaatggagttcattttttagccattcaatcttctcctgaatctcagtcctttgaggctttttggctacttttagaacagtcaagtaacaataattagcaaggggcaaaggcaattagtaattaaggtaaggcaatgggcaatagtgaataattaaaaattaaaaattaagaactaaaaactcctaactctcatcctcattacttattacccattactcccctaaacttgcaacctgccacctgaaacctgaaacctgaaacctctctataattattatcacacttattctactcaccatgaccgataaaattataaaaattgcagtaataggggatgttcacgatctttggaatgagtttgatcatgaggcgttggaatttttgcaagtggacttagtcttatttgtgggagattttggcaatgagtcaattcccttaattagtcgaattgctcaattaaatatacctaaagcaataatattaggtaatcatgatgcttggtttagtgccacggaatgggggagaaaaaaatgtccttacgatcgcaccttggaagacagagtacaacaacagttagacatactaggaaaatctcatgttggttatagctatcttgactttcccgaattagatatttctgtggtgggtagtcgcccttttagttgggggggttcaaaatggaaatgtgaagaattttatcgagaaaaatatgcgatcgaaaatttccatcagtcttcagacaaaatcatcgcctctgtaaataaagctcaagcccaacagataatttttgttggtcataatggaccttttggtttaggtgcaaatccagaagatacttgcggtagagattggaaacctttggggggagactttggcgatcctgattttcaagaagcgataaaatacactcaagaattaggcaaaaatgtaccattagtcacctttggtcatatgcaccatagcttaagacatactaaagatagattaagaaccattattaaccaagatgaacataaaacaatttatttgaacgccgcctccactcctcgtattcaagaaattgagggacaaaaaatccataaattttcccttgttaccctagatgcccgaatcgttaccaagattgatttaatcggtatcagtgaaaagatgaaaataaaagaagcaattaacctctatggtaaatgaattttaagaatagaaaggtcatcattaaaattactagaatggttgactttttctatctgcttaattaaatgttgcaattcgcctttatattttgcttgggtatccactaataaactaataaagttttctaacccccaaatattctcgttttcttgctcaatttcgtaaataccatcactgaaaatatataaataactatcgggtttaaccagacataaactctgatcaaattctacatcctcaatcatgcccactggtaaactaggagtctttaatttctcatatctccactgattgtcttcttgataaatcaatatagctggaggatgccccgccgaagaatatattagctcatgggtatcggtattatacaccccataccatatagtaaagtaattgaggcgatcgctatccatttgaaaaagacgatttaattctgtgataattgtccaaggttgataaggatttgtgttgtatagtgaattattacgaaccaaattaagaatagacacagataacaaagctgaatgaataccatgacctgcaacatctagtaaataaatagcgatattctcctcatccaaccaataataatcaaaaatatctcctcccaattttgaagatggaataaatttttgttctacggataaattatgttcctctggggaaggtaacaagatatcaatctagaatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctaatgcattttcagacaaaaataaattcgatgatcaaataaaaaaagtccttgtttaataagatttataataatttaagatccattccttgtaattgaatattaatagtaaactctaatgccagtatttgaaacggacaccgaagaaaaatcagagccagtatcagccaattcatcccttgctaatccttctcccacccatcataaagccaataacggaataatgagtgttttaaggaatattagttttcttattctctggctaggacaaattttttctcaattggcggataagatttatttagttttaatgatcgccttaattagtgctaactttcagactcaaggagaaagtatcagttcatgggtatcattaattatgatagcttttactatacctgcaattttatttggttctttggctggagtttatgttgatcgatggtcgaaaaaaagtgttttagttgtctctaatctcggtagaggtatattggttttaatcttgcctttttgcttgttaattaataaacaagaaatgggctttttctctttaccttggtcattttggttacttttattagttactttctccgtctccacattaactcaattttttgccccagccgaacaagcaacaattcctttaatcgtcagaaaaaaagacttattggcagcaaattctctatataccacaacaatgatggctatgttaatcattggttttgcagttggtgagcctttattggaaataacctataactggggtgagaatttttcttttgcttatggaaaagagttgctagtaggaggatgttatcttttggcgggaattgtattgatgacaatgcgcagtcgggaaaaggataaggatagacaacaacaagaaaatcatccttgggaagatataaaagatggtttacgatacttgcaaaaaaatcatcgagtgcgcaatgctttatttcaattagttattttattttctatttttgcggctttggcagtgttagcggtgcgtttagcggaaactatacccggaatggaggctgatcaatttggctatttattggccgcaacgggcattggcattgctattggtgcagtatttgttactcatcagggtaaaatgattcctcatgctcgacttagtttttggggttcaatgggtatgggagcggcattgagtggcttgtctgtggctactaacaatttgattttggctttactaatgacggttattttgggtatttttgcggctttggtgggtgtaccgatgcaaacgactattcaagcagaaaccccttctgatatgcgtggtaaggtttttggtttgcaaaataatgcggttaatattgctttgtctttacctttagctttagccggaattgcggaaacttattttggcttaaggattgttttgatgattcttgcctctttagctgtttcgggtatggctttaacttctttaattactggtaaatcggggaactaattgggtatggattgcttcatactaagtaatagatatttttatttttattgttactcaactatgttatttcatcaaagtaataggaaatttatgaaagtcggctctcaattttttttagtttgtggtttggctgtttttggtgttagtttggggattgttcctgagtacaatcagggttcaatttctattgaatctaaagctatggcgcaaaatgtttctgatgaagatttaaaaaaatatgcccaagctgcgatcgaaattgagaatttacgcaaaactacttatagtaatatagaaggaattgttggtaaatctatggggcaaatgagttgtaaccagcgacaaagtttcagtcaacttcctgataatgcccgtaacatggcgatcgaatattgtgatcagtcagaaactattgtcaaaaatcatggcttaagtgttaatcgttttaatcaaataactcagcaagtcaaacaaaatcccagtttaaaacaacgtttgcaatctattattggacaaatgtagtttagaagttattaattattcactccaacactatttacccttgccccttgccctttcttgattgctaaaaattataaacatcttcaataacccttacccacccctgtgctaaagaagctaaaatgcgatcgcctttttcctttgtagcggaggttgcatcccccatcacaccactatcgcttaactcttttgttaaccaagcaaagggtaattttccctccattgacaataaactgttgggtgcaagattacgagggtattctttgaccgctaaatccatttttacttgttggggcagtaaagctaacattaaactggtttcagcatccccagcgtgaattccccattcttgctcttcttccgttagcagttcgtttgtgatgtttggaactctccaagtaaaaaaaggaaaaacatctaaatcgggaaatttttgatgtaaatcacgggcaactatttccataacttggggttgtccgccgtgggagttcattaagattatttttctaaatccagctttgtaaatgctttctgccatttctaggattaaagtgtatagagtttgagaactaatcgtaatagttccagcaaatccatcgtgttcattagatttaccataataaagagtaggtaaagcaaaagcaggaattttgtcggatagtaacgccaacgctttccccaaaactccctcactaattgccgaatctactactaaaggtaaatgataaccgtgttgctcgatcgcacctatgggttgaataattactgtgttttccttattggacattttctcaatttcttgccatgttaaataggcaaaaaaacgatgaggaggtataaaaccatgaatcattttcaaaaaaaatattttatttttcagctttgttaccaatataacactacataatcacttttttcgagatagaataaagacctaaataaggatcattaaagggattaatttcgggataaaaaaagtggcattatcaaatcaactcgtcgacgaagttcctattctctagaaagtataggaacttccaaaaaacccctcaagacccgtttagaggccccaaggggttatgctagttaacattgaccagcaccctgactaggtttacaaacgtaaaaagtttctttaattccagtttttgcttcgtattgttctgcaacggcttgttgtactgcgggaactaactcttcaggaattaaagcaacaatacaaccaccaaaacctccaccagtcattctaacaccacctttgtcaccgattacagctttaacgatttctactaaggtgtcaatttgaggaacggtaatttcaaaatcatctctcatagaagcgtgagattcggccattaattctcccattctttttaagtctccttgttctaatgcacttgcggcttctacagttctggcgttttcggttaaaatatgtctaacacgtttagcaacgataggatctaattcatgggcaacggcattaaattcttcaatggtaacatctcttaaagcaggttgttggaaaaaacgggcaccagtttcacattgttcacgacgagtgttgtattcacttcctactaaagtacgtttaaaattagagttgatgataactactgcaacacctttaggcatagaaacggctttagtacctaaagaacgacaatcgattaataaagcatggtcttttttacctaaagcagagattaattgatccataataccacaattacaaccaacgaattgattctcggcctcttgaccatttaatgcgatttgggcaccatctaaaggtaaatgatataactgttgtaaaacggttccaacagctacctctaaagaagcactagaacttaaacctgctccttggggaacgttaccactgattaccatgtctacaccaccaaaagaattgtttcttaattgtaagtgtttaacaacaccacgtacataatttgcccactgatagttttcgtgagctacaatgggggcatctaaagaaaattcatctaattggttttcataatcggctgccataacacgaactttacggtcgtctctaggagcacaagaaattacagtttgatagtcgatagcacagggtaatacgaaaccatcattataatcagtatgctcaccaattaaattaacacgaccaggagcttgaatggtatgggtggcagggtatccaaaagcattagcgaataaactttgggttttctcttttaaaggcatgctaaactttttcctcatttttacctaatcttccataccatggtgacaggagaatatgaaatgaggatgaaatgatgctttaattattcatctgcttacttttccttacgaaccattgttgtaattcttcctcggtaattctgccactagctaaggtttggatgatatgtactacttccacttcagaagcagtaaagtgagaaccattgagcattaaaaaggttactccactaataaaacctgttcttttattaccatcaataaagggatgatttttaactatgctgaaggtataagtagaagctaaggatgagatattcctgcagtgagaaaaagtgtaaacaaatattaagaaaaagatcagaaaaatttaacaacacgtaataaaaaaatgcgtcactacgggttataaatttacatgaaaggttaaaacacttttctgagacgattttgata

SEQ ID NO: 2 - protein, 339 aa, Lyngbya sp.
MSETKFKAYAVMNPGEKLQPWEYEPAPLQVDEIEVRVTHNGLCHTDLHMRDNDWNVSEFPLVAGHEVVGEVTAVGEKVTSRKKGDRVGVGWIRNSCRACDHCLQGEENICREGYTGLIVGHHGGFADRVRVPADFTYKIPDALDSASAAPLLCAGITVYTPLRTYIKHPGMKVGVMGIGGLGHLAIKFARAMGAEVTAFSTSPNKEAQAKEFGAHHFQQWGTAEEMKAVAGNFDLVLSTISAETDWDAAFSLLANNGVLCFVGIPVSSLNVPLIPLIFGQKSVVGSVVGGRRFMAEMLEFAAVNQIKPMIETMPLSQVNEAMDKVAANKARYRIVLLSE

SEQ ID NO: 3 - protein, 271 aa, Artificial sequence
  Enzyme conferring kanamycin resistance- selectable marker on cloning vector
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 4 - protein, 569 aa, Zymomonas mobilis
MNSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGVRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 5 - DNA, 13080 bp, Artificial sequence
  Synthetic ethanologenic plasmid containing PnirA-pdc and PcpcB-adh
acaattaataacttcttcctgtacgggcgaatggccatttgctcctaactaactccgtactgctttgcggaacgagcgtagcgaactctccgaattactaagccttcatccctgatagatgcaaaaaacgaattaaaattatgtgtaaaaagaaaatgtgtctttatttagtagtcaaagttacaaaatattaagaatcaaattaataatgtattgggcagttaagtatataagtctttaaatatttatttgtattcaatatattaaggaggatcagccttatgaattcttataccgtgggtacttatttagccgaacgcttagtgcaaattggtttaaaacatcattttgccgtggctggggactataatttagtgttattggataacttattattaaataaaaacatggaacaagtgtattgttgtaatgaattaaattgtggtttttctgctgaaggttatgctagagctaaaggtgcagctgctgctgttgttacttattctgtgggtgctttatctgcttttgatgctattggtggtgcttatgccgaaaatttacccgtgattttaatttctggtgcccctaataataatgatcatgccgctggacatgttttacatcatgccttaggtaaaaccgattatcattatcaattagaaatggccaaaaatattactgctgctgccgaagctatttatactcctgaagaagcccctgccaaaattgatcatgtgattaaaaccgccttacgcgaaaaaaaacccgtgtatttagaaattgcctgtaatattgcttctatgccttgtgctgctcctgggcctgcttctgctttatttaatgatgaagcctctgatgaagctagtttaaatgctgccgtggaagaaaccttaaaatttattgccaatcgcgataaagttgccgtgttagttggttctaaattaagagctgctggtgctgaagaagctgctgttaaatttgctgatgctttaggtggtgcagttgctactatggctgctgccaaatctttttttcccgaagaaaatccccattatattggaactagttggggagaagtttcttatcctggtgtggaaaaaactatgaaagaagccgacgctgttattgctttagcccctgtgtttaatgattattctaccactggttggactgatattcccgatcccaaaaaattagttttagccgaacctcgttctgttgttgttaatggtgttcgctttccctctgtgcatttaaaagattatttaacccgcttagcccaaaaagtttctaaaaaaactggtgccttagatttttttaaatctttaaatgcgggtgaattaaaaaaagctgctcctgctgatccttctgctcctttagttaatgctgaaattgcccgtcaagttgaagccttattaacccctaatactaccgttattgccgaaactggtgattcttggtttaatgcccaacgcatgaaattacctaatggtgcccgtgttgaatatgaaatgcaatggggtcatattggttggtctgtacctgctgcttttggttatgctgttggtgctcctgaacgtcgtaatattttaatggtgggtgatggttcttttcaattaactgcccaagaagttgcccaaatggttcgcttaaaattacccgttattatttttttaataaataattatggttataccattgaagtgatgattcatgatgggccatataataatattaaaaattgggattatgcgggtttaatggaagtgtttaatggtaatggtggttatgattctggtgctggtaaaggtttaaaagccaaaactggtggtgaattagctgaagctattaaagttgccttagccaatactgatgggccaaccttaattgaatgttttattggtcgcgaagattgtaccgaagaattagttaaatggggtaaacgtgttgctgctgctaattctcgcaaacccgtgaataaattattgtaaggatccagcaaggtttcatcccgaccccctcagggtcgggatttttttattgtgagctcaactttagatattcgtagttggcaatgtcgtaaatgcggaacaatacatggaaaacatatagatttgtaatgagaaaaagtgtaaacaaatattaagaaaaagatcagaaaaatttaacaacacgtaataaaaaaatgcgtcactacgggttataaatttacatgaaaggttaaaacacttttctgagacgattttgataaaaaagttgtcaaaaaattaagtttctttacaaatgcttaacaaaaacttggttttaagcacaaaataagagagactaatttgcagaagttttacaaggaaatcttgaagaaaaagatctaagtaaaacgactctgtttaaccaaaatttaacaaatttaacaaaacaaactaaatctattaggagattaactacatatgagtgaaactaaatttaaagcctatgccgtaatgaatcctggtgaaaaattacaaccctgggaatatgaacctgctcctttacaggtagatgaaattgaagtaagagttactcacaatggtttatgtcacactgacttacacatgagagataatgactggaatgttagtgagttccccttagtagcaggtcatgaagttgttggtgaagtaaccgctgttggtgaaaaagtaaccagtcgtaaaaaaggtgatagagttggtgtaggttggattcgtaattcttgtcgcgcttgtgaccattgtttacaaggagaagagaacatttgtagagagggttatactggtttaattgttggtcatcacggtggatttgctgatcgtgtacgtgtacctgctgacttcacttataaaattcctgatgctttagatagtgcatctgctgctcctttattatgtgccggtattaccgtttacactcctttaagaacctacattaaacatcccggtatgaaagtaggtgttatgggtattggaggattaggacatttagctattaaatttgctcgtgcaatgggagcagaagttactgcctttagtaccagtcctaataaagaagcccaagccaaagaatttggtgctcatcatttccaacaatggggtactgctgaagaaatgaaagctgttgccggtaattttgatttagttttatctaccatctctgctgaaactgactgggatgctgccttctctttattagcaaataacggtgttttatgtttcgtaggtattcccgttagttctttaaatgttcctttaattcctttaattttcggacaaaaatctgttgtaggttctgtagttggaggaagaagattcatggcagaaatgttagagttcgccgctgtaaatcagattaaacctatgatcgaaactatgcccttatctcaagtaaatgaagctatggataaagttgccgccaataaagccagatatagaattgtattattatctgaataactagatctacttctaaactgaaacaaatttgagggtaggcttcattgtctgcccttatttttttatttaggaaaagtgaacagactaaagagtgttggctctattgctttgagtatgtaaattaggcgttgctgaattaaggtatgatttttgacccctgcaggatcatcttgctgaaaaactcgagcgctcgttccgcaaagcggtacggagttagttaggggctaatgggcattctcccgtacaggaaagagttagaagttattaattatcaacaattctcctttgcctagtgcatcgttacctttttaattaaaacataaggaaaactaataatcgtaataatttaacctcaaagtgtaaagaaatgtgaaattctgacttttataacgttaaagagggaaaaattagcagtttaaaatacctagagaatagtctggggtaagcatagagaattagattagttaagttaatcaaattcagaaaaaataataatcgtaaatagttaatctgggtgtatagaaaatgatccccttcatgataagatttaaactcgaaaagcaaaagccaaaaaactaacttccattaaaagaagttgttacatataacgctataaagaaaatttatatatttggaggataccaaccatgtctcatattcaacgtgaaactagttgttctcgccctcgtttaaattctaatatggatgccgatttatatggttataaatgggctcgtgataatgttggtcaatctggtgctactatttatcgtttatatggtaaacctgatgctcctgaattattcttgaaacatggtaaaggttctgttgctaatgatgttactgatgaaatggttcgtttaaactggttgactgaatttatgcctttacctactattaaacattttattcgtactcccgatgatgcttggttattaactactgctattcctggtaaaactgcttttcaagttttagaagaatatcctgattctggtgaaaatattgttgatgctttagctgtttttttacgtcgtttacattctattcccgtttgtaattgtccttttaattctgatcgtgtttttcgtttagctcaagctcaatctcgtatgaataatggtttagttgatgcttctgattttgatgatgaacgtaatggttggcctgttgaacaagtttggaaagaaatgcacaaattgttacctttttctcctgattctgttgttactcatggtgatttttctttagataatttgatctttgatgaaggtaaattgattggttgtattgatgttggtcgtgttggtattgctgatcgttatcaagatttagctattttatggaattgtttaggtgaattttctccttctttacagaaacgtttatttcagaaatatggtattgataatcctgatatgaacaagttacaatttcatttaatgttggacgagttcttttaagaattaattcatgaccaaaatcccttaacgtgagttttcgttccactgagcgtcagaccccgtagaaaagatcaaaggatcttcttgagatcctttttttctgcgcgtaatctgctgctatttaaattacgtacacgtgttattactttgttaacgacaattgtcttaattaactgggcctcatgggccttccgctcactgcccgctttccagtcgggaaacctgtcgtgccagctctgcagatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgcagaagcttgttagacaccctgtcatgtattttatattatttatttcaccatacggattaagtgaaacctaatgaaaatagtactttcggagctttaactttaatgaaggtatgtttttttatagacatcgatgtctggtttaacaataggaaaaagtagctaaaactcccatgaattaaagaaataacaaggtgtctaacaacctgttattaagaatgttagaaaagacttaacatttgtgttgagtttttatagacattggtgtctagacatacggtagataaggtttgctcaaaaataaaataaaaaaagattggactaaaaaacatttaatttagtacaatttaattagttattttttcgtctcaaattttgctttgttgagcagaaatttagataaaaaaatccccgtgatcagattacaatgtcgttcattgtacgatgtgtcgaaaaatctttacgacactctaaactgaccacacgggggaaaaagaaaactgaactaataacatcatgatactcggaaaacctagcaattctcaacccctaaacaaaagaaacttccaaaaccctgaccatataaaggagtggcaacaatcagcaatcagtcaagatttgatagcagaaaatcttgtatcggttgctaatggttttgatgtactatttatcggcaataaataccgaactaacacgggtgttctgtcacggcacatattaaactcctattctcatttagaagatggtggttcgtatggtagaacatttgacccatttaccaataaagaaatgcagtgggttcaatttaaaccgaatagaccaagaaaaggttctactggtaaggtaatcaaatatgaatcgccaaaaggtgaacctacaagagttctaatgccgtttgtgcctatgaaaatatggcaacggattagcgataagttcggagtaccgattaatccgaaaaaagatactcacttttgggaatgggtaaagaataatccatcgataccgattgccattacagaaggaaataaaaaagctaattgcctattatcctatggctatcctgctattgcctttgtaggcatttggaacggattagagaaaataaatgatttctcgaaggaaaagcagttaaaagaggatttgaaatggttgttatccaacggcaaccgaaatattaatatcatctttgaccaagaccagaaacaaaaaactgtaattaatgtaaacaaagctattttcgctttatcttctctaataagtagaaatggtcataaagttaatattgtgcaatggttgccgtcaaaaggtaaaggaatagatgattatttggtagctttaccttttgagaaaagagaaaatcatttagacaacttaattaaaattgcaccatcatttaatttttggtcaactaaatacttattcaagtgtcgtaaaccagatttaaccgtaaattgccgttatttgagcgatgcagtaaaagaattacctcaagaggatatagcattaatagcacctcacggcacgggtaaaacttcattagtagctactcacgttaagaatcggagttatcacggaaggaaaactatttcattggtgcatcttgaaagtttagccaaagctaatggcaacgcacttggattatattaccgaaccgaaaataatattgaaaagcaatatcttggatttagcttatgtgtagatagttgccgtgataagattaacggcattacaactgatattatttcaggtcaagattattgccttttcattgatgaaattgaccaagtaattccacacatccttaacagtgaaactgaagtaagtaagtatagatgcaccatcattgacactttttctgaactggtgagaaatgctgaacaggtcattattgctgatgctgatttatccgatgtgacgattgacctaatagaaaacatcagaggtaaaaaactatatgtaatcaagaatgaatatcagtatcagggaatgacttttaacgccgttggttcaccattagaaatgatggcaatgatgggaaaatcggtgtcagaaggcaagaaattatttattaacaccacatcccaaaaggcaaaaagtaagtacggcacaatcgctcttgagtcttatatttttggtctaaataaagaagcaaagatattaagaatagactctgaaaccactaaaaaccctgaacatccagcctataaaatcattgaccaagacttaaataatatcctcaaagattatgattatgtcattgcctcaccttgccttcaaacaggtgtcagtattaccttaaaagggcattttgaccagcaatttaacttttccagtggaaacattacacctcattgctttttacagcaaatgtggcggttgagggatgcagaaattgaaagattctattatgtgccgaactcatctaacctcaatctcattgggaataagtcaagttcaccatcagaccttctaaagagcaataacaagatggcaacggcaacggttaaccttttgggtagaatcgactccgaatattccctagagtatgaatcgcacggcatttggcttgagacgtgggcaaaattatcagcacggcataacagttcaatgcgttgttactctgaaattcttacctatctaattacgtctcaagggcataaattaaatatcaacattccctcacctcttgcagatattaagaagctaaatgatgaggtaagtagtaacagggaaaaggtaaaaaatgagagatactctcagaggttaaactcaccagatattaacgatgcagaagctaccatactcgaatctaaagagcaaaaaatcggattgactctcaatgagagatgcaccctagaaaagcataaagttaagaagcggtatgggaatgtaaagatggatattctcacctttgatgatgatggactataccccaaactcagactattttattacctcaccatcggtaaacctcatctcaaggctaatgacagaaaagctattgccaaaatgggcaatgacaataaaggcaagattctatcaaaagacttagttaataaaacttactccgctcgtgtgaaggtcttagagattcttaaactaactgactttatcgacaatcttagagatgaactcttaataactcccaataatccagctatcaccgattttaataatcttctgctaagagctaagaaggatttaagagtattaggagtcaacatcggaaaatatccaatggccaacattaatgccgtacttactctcattggtcacaaactttctgtaatgagagatgagttcggaaaagagaaaaggataaaagtagatggtaaatcataccgatgttatcaacttgaaacattaccagattttaccaatgatactcttgactactggttagaaaatgatagccaaaaagaagtaacagcaacagaaaattactccgaaaattttaacccttcaaatagctacaatccagacagtaagacactttcagagggtgcaaatttcctatatataaataaagaagaattgcatccaaataaattgcacctagaaataaaagaaggtgctgaactttttttattcggggtaaaggtgattgtgaaaggaatcttggacggggcagtaactatattctctatgggtcaagaatacgatttatccctcaatgaactagaggggatgttaacatcatgaactttacaagaatctttttaaagggcgatcgcaccatgttaaatgatggtacatttgttcagatatttgatatttaccatgaccacgcattgggagtgacccttgaccttaagacagaaaaaattatttccgatgatgttagggtaattactgtcaaagacttattgttcgatggcacttataaaggggtaaaatcttttatgcccgataatgcccgataatgcccgattgatgctacaaaatcccataatcataagcgataatcccctaatagcttgtaattcttgaaccgtagcgattttagagtattccaaaaagaagaaataaacaccgcaaaatgtcgtatttcacatatataaaccaaggttttttgccctaaaatctttatgtttgtagtgtgatgttgggtcaaaatggtcagaaaagttgcaaggtttttatggatgcttacgcgcgcgaggggtaagcatccccaaatagttactttatcctagtccatgcccatttattgccgtcccgttcggctttaaaaaagtgccaaaactcacaaggtgcaataaaaagttctgtacctttcgcaaccctagataatctttcaacagttactttttttcctattatctcggtacaaagtttggctagtttctcttttccctctttttcaatcaagccttcttgtatgcccaactcattgattaatctctctatttttaccattatttcccgttcaggtagtttatcccctaaatcttcatcggggggcaatgtagggcattctgaaggggctttttcttctgtctggacattatctaatattgaagtaaccaaactatcttcagttttttctattcctattaattcatattcggttactgtatccgtatcaatatccgaataactatctttatccgtattagctattcggttaagtttatccgttaactcagaaacaagactatatagcggttttagcttttcttctatcctgttatctaatacggataagtttatacggttatcattatccgtattagtatcattgggcttttttggtagttctaccccctcataaaccgcttttattcccaattccaacagactgataacagtatcctttataatgggttttttgctgatatggtgaacttttgccccttccatcattgcgatactttctatctcactcatcaacttatcgcttaagtgaatctcgtatctgtttaatcccttactggttttattcatatccgtttactttattcggttaacaattctattttatacgaataaaatattatacggttaactttatacgtttaactattttatctatacggataacagtaataagttattcgtattagttatacgtttacttttatccaaataaaattagtgcatttaaactaaaagaatgattttatcggagttgatagcattggattaacctaaagatgtttataagctatatctgataagtatttaaggttattttgttattctgtttattgacattatcagaataaaagaatagaatataattgttgagagataagaggtttaagtgattatggttaagaagttagttggttatgtcagggtcagtagtgaatcgcaagaggataacactagcttacagaatcagatagagagaattgaagcatattgtatggcttttggttatgagttggtaaaaatattcaaagaggttgccactggtacaaaagcagatattgaaacccgtcctatttttaatgaagctatagaatacttgaaacaggataatgctaatggaattattgccttgaagctagaccgaatcgcacggaatgctttagatgtattgcgtttggttcgtgaaaccttagaaccacaaaataaaatgttagtgttactagatattcaggtagatacttcgacaccttcaggaaaaatgattttaactgtaatgagtgccgttgctgaactcgaaagagacatgatctatgatcgcactcaggggggtagaaagactaaagcccaaaagggcgggtatgcctacgggaaacctaaatttggctataagactgaagaaaaggaactaaaagaagattcagcacaacaggaaactattaaactaattaagagacaccgtaggtcagggaaaagctaccagaaaatagctgattatctcaatgcccaaagtattcccactaaacaaggtaagaaatggagttctagcgtcgtctatcgaatctgtcaggaaaaagctggttaagtctgtttatagatatttagaatttattgaataaaaatagtatgaacaataaatatttatggactaaccacgctcggaaacgtttaactgaacgatgggaaataaaagaatcatgggttattgataccatcgaaaatcctgaacgttcagaatttattgttgatgagtcaggggaaaaatatcattactataaaagaatagctaagtttaagaatagagtgttagaagtgataacttctgccaactcaacacccacaagaataataaccttttactttaaccgtaacatgaggaaaaatttatgattgttacttacgataatgaagttgacgcaatttattttaagttaacggaaaataaaattgatagcaccgaacctcaaacagacaggattatcattgattacgatgaaagtaataatattgttggcattgaggtattagattttaattatcttgtcaagaaaggtttaaccgttgctgatttacctttttctgaagatgaaagattaacagcttctcaatattttaattttcctgttgctatctaatccagaaggggcaataatccccttctttcatcgagttagacttaatatcacaaaagtcattttcattttaccgtttcttttccacagcgtccgtacgcccctcgttaaatctcaaaaccgacaatttatgatgtttataaaaagttactcactttaataagtatttatactcattaaagggttattctttttttgtagcctgataggttgggaaggaatatttcagattatcagatttgttgaatatttttcgtcagatacgcaaaccttacaaacataattaacaactgaaactattgatatgtctaggttttagctctatcacaggttggatctgtcg

SEQ ID NO: 6 - protein, 568 aa, Zymomonas mobilis
NSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGVRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 7 - protein, 336 aa, Synechocystis sp. PCC 6803
MIKAYAALEANGKLQPFEYDPGALGANEVEIEVQYCGVCHSDLSMINNEWGISNYPLVPGHEVVGTVAAMGEGVNHVEVGDLVGLGWHSGYCMTCHSCLSGYHNLCATAESTIVGHYGGFGDRVRAKGVSVVKLPKGIDLASAGPLFCGGITVFSPMVELSLKPTAKVAVIGIGGLGHLAVQFLRAWGCEVTAFTSSARKQTEVLELGAHHILDSTNPEAIASAEGKFDYIISTVNLKLDWNLYISTLAPQGHFHFVGVVLEPLDLNLFPLLMGQRSVSASPVGSPATIATMLDFAVRHDIKPVVEQFSFDQINEAIAHLESGKAHYRVVLSHSKN

SEQ ID NO: 8 - protein, 271 aa, Artificial sequence; identical to SEQ ID NO: 3
  Enzyme conferring kanamycin resistance_ selectable marker on cloning vector

SEQ ID NO: 9 - DNA, 12657 bp, Artificial sequence
  Synthetically produced plasmid construct with pdc and adh genes
tcgacaattaataacttcttcctgtacgggcgaatggccatttgctcctaactaactccgtactgctttgcggaacgagcgtagcgaactctccgaattactaagccttcatccctgatagatgcaaaaaacgaattaaaattatgtgtaaaaagaaaatgtgtctttatttagtagtcaaagttacaaaatattaagaatcaaattaataatgtattgggcagttaagtatataagtctttaaatatttatttgtattcaatatattaaggaggatcagccttatgaattcttataccgtgggtacttatttagccgaacgcttagtgcaaattggtttaaaacatcattttgccgtggctggggactataatttagtgttattggataacttattattaaataaaaacatggaacaagtgtattgttgtaatgaattaaattgtggtttttctgctgaaggttatgctagagctaaaggtgcagctgctgctgttgttacttattctgtgggtgctttatctgcttttgatgctattggtggtgcttatgccgaaaatttacccgtgattttaatttctggtgcccctaataataatgatcatgccgctggacatgttttacatcatgccttaggtaaaaccgattatcattatcaattagaaatggccaaaaatattactgctgctgccgaagctatttatactcctgaagaagcccctgccaaaattgatcatgtgattaaaaccgccttacgcgaaaaaaaacccgtgtatttagaaattgcctgtaatattgcttctatgccttgtgctgctcctgggcctgcttctgctttatttaatgatgaagcctctgatgaagctagtttaaatgctgccgtggaagaaaccttaaaatttattgccaatcgcgataaagttgccgtgttagttggttctaaattaagagctgctggtgctgaagaagctgctgttaaatttgctgatgctttaggtggtgcagttgctactatggctgctgccaaatctttttttcccgaagaaaatccccattatattggaactagttggggagaagtttcttatcctggtgtggaaaaaactatgaaagaagccgacgctgttattgctttagcccctgtgtttaatgattattctaccactggttggactgatattcccgatcccaaaaaattagttttagccgaacctcgttctgttgttgttaatggtgttcgctttccctctgtgcatttaaaagattatttaacccgcttagcccaaaaagtttctaaaaaaactggtgccttagatttttttaaatctttaaatgcgggtgaattaaaaaaagctgctcctgctgatccttctgctcctttagttaatgctgaaattgcccgtcaagttgaagccttattaacccctaatactaccgttattgccgaaactggtgattcttggtttaatgcccaacgcatgaaattacctaatggtgcccgtgttgaatatgaaatgcaatggggtcatattggttggtctgtacctgctgcttttggttatgctgttggtgctcctgaacgtcgtaatattttaatggtgggtgatggttcttttcaattaactgcccaagaagttgcccaaatggttcgcttaaaattacccgttattatttttttaataaataattatggttataccattgaagtgatgattcatgatgggccatataataatattaaaaattgggattatgcgggtttaatggaagtgtttaatggtaatggtggttatgattctggtgctggtaaaggtttaaaagccaaaactggtggtgaattagctgaagctattaaagttgccttagccaatactgatgggccaaccttaattgaatgttttattggtcgcgaagattgtaccgaagaattagttaaatggggtaaacgtgttgctgctgctaattctcgcaaacccgtgaataaattattgtaatttttggggatcaattcgagctcagcaagtttcatcccgaccccctcagggtcgggatttttttattgtactagttgacataagtaaaggcatcccctgcgtgatataattaccttcagtttaaggaggtatacacatatgattaaagcctacgctgccctggaagccaacggaaaactccaaccctttgaatacgaccccggtgccctgggtgctaatgaggtggagattgaggtgcagtattgtggggtgtgccacagtgatttgtccatgattaataacgaatggggcatttccaattaccccctagtgccgggtcatgaggtggtgggtactgtggccgccatgggcgaaggggtgaaccatgttgaggtgggggatttagtggggctgggttggcattcgggctactgcatgacctgccatagttgtttatctggctaccacaacctttgtgccacggcggaatcgaccattgtgggccactacggtggctttggcgatcgggttcgggccaagggagtcagcgtggtgaaattacctaaaggcattgacctagccagtgccgggccccttttctgtggaggaattaccgttttcagtcctatggtggaactgagtttaaagcccactgcaaaagtggcagtgatcggcattgggggcttgggccatttagcggtgcaatttctccgggcctggggctgtgaagtgactgcctttacctccagtgccaggaagcaaacggaagtgttggaattgggcgctcaccacatactagattccaccaatccagaggcgatcgccagtgcggaaggcaaatttgactatattatctccactgtgaacctgaagcttgactggaacttatacatcagcaccctggcgccccagggacatttccactttgttggggtggtgttggagcctttggatctaaatctttttccccttttgatgggacaacgctccgtttctgcctccccagtgggtagtcccgccaccattgccaccatgttggactttgctgtgcgccatgacattaaacccgtggtggaacaatttagctttgatcagatcaacgaggcgatcgcccatctagaaagcggcaaagcccattatcgggtagtgctcagccatagtaaaaattagctctgcaaaggttgcttctgggtccgtggaacgctcggttgccgccgggcgttttttattcctgcaggatcatcttgctgaaaaactcgagcgctcgttccgcaaagcggtacggagttagttaggggctaatgggcattctcccgtacaggaaagagttagaagttattaattatcaacaattctcctttgcctagtgcatcgttacctttttaattaaaacataaggaaaactaataatcgtaataatttaacctcaaagtgtaaagaaatgtgaaattctgacttttataacgttaaagagggaaaaattagcagtttaaaatacctagagaatagtctggggtaagcatagagaattagattagttaagttaatcaaattcagaaaaaataataatcgtaaatagttaatctgggtgtatagaaaatgatccccttcatgataagatttaaactcgaaaagcaaaagccaaaaaactaacttccattaaaagaagttgttacatataacgctataaagaaaatttatatatttggaggataccaaccatgtctcatattcaacgtgaaactagttgttctcgccctcgtttaaattctaatatggatgccgatttatatggttataaatgggctcgtgataatgttggtcaatctggtgctactatttatcgtttatatggtaaacctgatgctcctgaattattcttgaaacatggtaaaggttctgttgctaatgatgttactgatgaaatggttcgtttaaactggttgactgaatttatgcctttacctactattaaacattttattcgtactcccgatgatgcttggttattaactactgctattcctggtaaaactgcttttcaagttttagaagaatatcctgattctggtgaaaatattgttgatgctttagctgtttttttacgtcgtttacattctattcccgtttgtaattgtccttttaattctgatcgtgtttttcgtttagctcaagctcaatctcgtatgaataatggtttagttgatgcttctgattttgatgatgaacgtaatggttggcctgttgaacaagtttggaaagaaatgcacaaattgttacctttttctcctgattctgttgttactcatggtgatttttctttagataatttgatctttgatgaaggtaaattgattggttgtattgatgttggtcgtgttggtattgctgatcgttatcaagatttagctattttatggaattgtttaggtgaattttctccttctttacagaaacgtttatttcagaaatatggtattgataatcctgatatgaacaagttacaatttcatttaatgttggacgagttcttttaagaattaattcatgaccaaaatcccttaacgtgagttttcgttccactgagcgtcagaccccgtagaaaagatcaaaggatcttcttgagatcctttttttctgcgcgtaatctgctgctatttaaattacgtacacgtgttattactttgttaacgacaattgtcttaattaactgggcctcatgggccttccgctcactgcccgctttccagtcgggaaacctgtcgtgccagctctgcagatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgcagaagcttgttagacaccctgtcatgtattttatattatttatttcaccatacggattaagtgaaacctaatgaaaatagtactttcggagctttaactttaatgaaggtatgtttttttatagacatcgatgtctggtttaacaataggaaaaagtagctaaaactcccatgaattaaagaaataacaaggtgtctaacaacctgttattaagaatgttagaaaagacttaacatttgtgttgagtttttatagacattggtgtctagacatacggtagataaggtttgctcaaaaataaaataaaaaaagattggactaaaaaacatttaatttagtacaatttaattagttattttttcgtctcaaattttgctttgttgagcagaaatttagataaaaaaatccccgtgatcagattacaatgtcgttcattgtacgatgtgtcgaaaaatctttacgacactctaaactgaccacacgggggaaaaagaaaactgaactaataacatcatgatactcggaaaacctagcaattctcaacccctaaacaaaagaaacttccaaaaccctgaccatataaaggagtggcaacaatcagcaatcagtcaagatttgatagcagaaaatcttgtatcggttgctaatggttttgatgtactatttatcggcaataaataccgaactaacacgggtgttctgtcacggcacatattaaactcctattctcatttagaagatggtggttcgtatggtagaacatttgacccatttaccaataaagaaatgcagtgggttcaatttaaaccgaatagaccaagaaaaggttctactggtaaggtaatcaaatatgaatcgccaaaaggtgaacctacaagagttctaatgccgtttgtgcctatgaaaatatggcaacggattagcgataagttcggagtaccgattaatccgaaaaaagatactcacttttgggaatgggtaaagaataatccatcgataccgattgccattacagaaggaaataaaaaagctaattgcctattatcctatggctatcctgctattgcctttgtaggcatttggaacggattagagaaaataaatgatttctcgaaggaaaagcagttaaaagaggatttgaaatggttgttatccaacggcaaccgaaatattaatatcatctttgaccaagaccagaaacaaaaaactgtaattaatgtaaacaaagctattttcgctttatcttctctaataagtagaaatggtcataaagttaatattgtgcaatggttgccgtcaaaaggtaaaggaatagatgattatttggtagctttaccttttgagaaaagagaaaatcatttagacaacttaattaaaattgcaccatcatttaatttttggtcaactaaatacttattcaagtgtcgtaaaccagatttaaccgtaaattgccgttatttgagcgatgcagtaaaagaattacctcaagaggatatagcattaatagcacctcacggcacgggtaaaacttcattagtagctactcacgttaagaatcggagttatcacggaaggaaaactatttcattggtgcatcttgaaagtttagccaaagctaatggcaacgcacttggattatattaccgaaccgaaaataatattgaaaagcaatatcttggatttagcttatgtgtagatagttgccgtgataagattaacggcattacaactgatattatttcaggtcaagattattgccttttcattgatgaaattgaccaagtaattccacacatccttaacagtgaaactgaagtaagtaagtatagatgcaccatcattgacactttttctgaactggtgagaaatgctgaacaggtcattattgctgatgctgatttatccgatgtgacgattgacctaatagaaaacatcagaggtaaaaaactatatgtaatcaagaatgaatatcagtatcagggaatgacttttaacgccgttggttcaccattagaaatgatggcaatgatgggaaaatcggtgtcagaaggcaagaaattatttattaacaccacatcccaaaaggcaaaaagtaagtacggcacaatcgctcttgagtcttatatttttggtctaaataaagaagcaaagatattaagaatagactctgaaaccactaaaaaccctgaacatccagcctataaaatcattgaccaagacttaaataatatcctcaaagattatgattatgtcattgcctcaccttgccttcaaacaggtgtcagtattaccttaaaagggcattttgaccagcaatttaacttttccagtggaaacattacacctcattgctttttacagcaaatgtggcggttgagggatgcagaaattgaaagattctattatgtgccgaactcatctaacctcaatctcattgggaataagtcaagttcaccatcagaccttctaaagagcaataacaagatggcaacggcaacggttaaccttttgggtagaatcgactccgaatattccctagagtatgaatcgcacggcatttggcttgagacgtgggcaaaattatcagcacggcataacagttcaatgcgttgttactctgaaattcttacctatctaattacgtctcaagggcataaattaaatatcaacattccctcacctcttgcagatattaagaagctaaatgatgaggtaagtagtaacagggaaaaggtaaaaaatgagagatactctcagaggttaaactcaccagatattaacgatgcagaagctaccatactcgaatctaaagagcaaaaaatcggattgactctcaatgagagatgcaccctagaaaagcataaagttaagaagcggtatgggaatgtaaagatggatattctcacctttgatgatgatggactataccccaaactcagactattttattacctcaccatcggtaaacctcatctcaaggctaatgacagaaaagctattgccaaaatgggcaatgacaataaaggcaagattctatcaaaagacttagttaataaaacttactccgctcgtgtgaaggtcttagagattcttaaactaactgactttatcgacaatcttagagatgaactcttaataactcccaataatccagctatcaccgattttaataatcttctgctaagagctaagaaggatttaagagtattaggagtcaacatcggaaaatatccaatggccaacattaatgccgtacttactctcattggtcacaaactttctgtaatgagagatgagttcggaaaagagaaaaggataaaagtagatggtaaatcataccgatgttatcaacttgaaacattaccagattttaccaatgatactcttgactactggttagaaaatgatagccaaaaagaagtaacagcaacagaaaattactccgaaaattttaacccttcaaatagctacaatccagacagtaagacactttcagagggtgcaaatttcctatatataaataaagaagaattgcatccaaataaattgcacctagaaataaaagaaggtgctgaactttttttattcggggtaaaggtgattgtgaaaggaatcttggacggggcagtaactatattctctatgggtcaagaatacgatttatccctcaatgaactagaggggatgttaacatcatgaactttacaagaatctttttaaagggcgatcgcaccatgttaaatgatggtacatttgttcagatatttgatatttaccatgaccacgcattgggagtgacccttgaccttaagacagaaaaaattatttccgatgatgttagggtaattactgtcaaagacttattgttcgatggcacttataaaggggtaaaatcttttatgcccgataatgcccgataatgcccgattgatgctacaaaatcccataatcataagcgataatcccctaatagcttgtaattcttgaaccgtagcgattttagagtattccaaaaagaagaaataaacaccgcaaaatgtcgtatttcacatatataaaccaaggttttttgccctaaaatctttatgtttgtagtgtgatgttgggtcaaaatggtcagaaaagttgcaaggtttttatggatgcttacgcgcgcgaggggtaagcatccccaaatagttactttatcctagtccatgcccatttattgccgtcccgttcggctttaaaaaagtgccaaaactcacaaggtgcaataaaaagttctgtacctttcgcaaccctagataatctttcaacagttactttttttcctattatctcggtacaaagtttggctagtttctcttttccctctttttcaatcaagccttcttgtatgcccaactcattgattaatctctctatttttaccattatttcccgttcaggtagtttatcccctaaatcttcatcggggggcaatgtagggcattctgaaggggctttttcttctgtctggacattatctaatattgaagtaaccaaactatcttcagttttttctattcctattaattcatattcggttactgtatccgtatcaatatccgaataactatctttatccgtattagctattcggttaagtttatccgttaactcagaaacaagactatatagcggttttagcttttcttctatcctgttatctaatacggataagtttatacggttatcattatccgtattagtatcattgggcttttttggtagttctaccccctcataaaccgcttttattcccaattccaacagactgataacagtatcctttataatgggttttttgctgatatggtgaacttttgccccttccatcattgcgatactttctatctcactcatcaacttatcgcttaagtgaatctcgtatctgtttaatcccttactggttttattcatatccgtttactttattcggttaacaattctattttatacgaataaaatattatacggttaactttatacgtttaactattttatctatacggataacagtaataagttattcgtattagttatacgtttacttttatccaaataaaattagtgcatttaaactaaaagaatgattttatcggagttgatagcattggattaacctaaagatgtttataagctatatctgataagtatttaaggttattttgttattctgtttattgacattatcagaataaaagaatagaatataattgttgagagataagaggtttaagtgattatggttaagaagttagttggttatgtcagggtcagtagtgaatcgcaagaggataacactagcttacagaatcagatagagagaattgaagcatattgtatggcttttggttatgagttggtaaaaatattcaaagaggttgccactggtacaaaagcagatattgaaacccgtcctatttttaatgaagctatagaatacttgaaacaggataatgctaatggaattattgccttgaagctagaccgaatcgcacggaatgctttagatgtattgcgtttggttcgtgaaaccttagaaccacaaaataaaatgttagtgttactagatattcaggtagatacttcgacaccttcaggaaaaatgattttaactgtaatgagtgccgttgctgaactcgaaagagacatgatctatgatcgcactcaggggggtagaaagactaaagcccaaaagggcgggtatgcctacgggaaacctaaatttggctataagactgaagaaaaggaactaaaagaagattcagcacaacaggaaactattaaactaattaagagacaccgtaggtcagggaaaagctaccagaaaatagctgattatctcaatgcccaaagtattcccactaaacaaggtaagaaatggagttctagcgtcgtctatcgaatctgtcaggaaaaagctggttaagtctgtttatagatatttagaatttattgaataaaaatagtatgaacaataaatatttatggactaaccacgctcggaaacgtttaactgaacgatgggaaataaaagaatcatgggttattgataccatcgaaaatcctgaacgttcagaatttattgttgatgagtcaggggaaaaatatcattactataaaagaatagctaagtttaagaatagagtgttagaagtgataacttctgccaactcaacacccacaagaataataaccttttactttaaccgtaacatgaggaaaaatttatgattgttacttacgataatgaagttgacgcaatttattttaagttaacggaaaataaaattgatagcaccgaacctcaaacagacaggattatcattgattacgatgaaagtaataatattgttggcattgaggtattagattttaattatcttgtcaagaaaggtttaaccgttgctgatttacctttttctgaagatgaaagattaacagcttctcaatattttaattttcctgttgctatctaatccagaaggggcaataatccccttctttcatcgagttagacttaatatcacaaaagtcattttcattttaccgtttcttttccacagcgtccgtacgcccctcgttaaatctcaaaaccgacaatttatgatgtttataaaaagttactcactttaataagtatttatactcattaaagggttattctttttttgtagcctgataggttgggaaggaatatttcagattatcagatttgttgaatatttttcgtcagatacgcaaaccttacaaacataattaacaactgaaactattgatatgtctaggttttagctctatcacaggttggatctg

SEQ ID NO: 10 - protein, 380 aa, Cyanobacterium sp.
MKTKQLFGSVSALFVALSLTACGGGGQPTENTTGGGETTGGGETASSTTIEVPFEGTVSLNGAGASFPAPLYQNWFVSLAQVQPKLQVNYQSVGSGAGVEQFTQQTVDFGASDVAMKDEEIDAIDRGVLLLPMTAGSIVYAYNLPGVEGLQLSRETYVDITLGKITNWNDPKIAADNPDLTLPDLPITFVHRSDGSGTTGVFTMHLSAISPEWKDTIGEGKTVQWPSTGNFIGGKGNEGVTAAISQTEGAIGYVEYGYAKNNGLTMASLQNQAGNFIVPSDESASKTLANVELPENLRAFITDPEGDESYPIVTYTWMMAYKTYDDPNKAIAMEAMIQYGLTEGQKAAPELGYIPLPANVVEKVAAAADQITPDYTITVN

SEQ ID NO: 11 - protein, 342 aa, Cyanobacterium sp.
MTDFQIRRRDFLYGLAGLSGSVIASQLPFESASAQAMRLNGAGASFPAPLYQRWFVEYNKVNRNVQVNYQSVGSGAGVQQFMGGTVDFGASDVAMKDEEISKVSRGVVLLPVTAGSIVMAYNNKDVGTLKLSRQQLVDVLLGKIKDWKQLGASKSKPIRVVYRSDGSGTTGVFTKHLSAISSEWKSKVGEGKSVQWPVGVGAKGNEGVTATVMQTDGAIGYVEYGFAKNNNLKTAQLQNKAGKYITPTLQNASISLSQVALPANLRAFIPDPSGDGSYPIVSYSWILAYKKYPDAKKAAALKNVLKWCVSKTGGQKYSEQLGYVALPDNVIQKVTQAINTIA

SEQ ID NO: 12 - protein, 361 aa, Cyanobacterium sp.
MGKFKSLIPSALGGLLFLSAGLTGCGNKSTSSVSAPSVDPNQIVMLKGSGATFPAPLYQKWIQEYSSTDDKKNIRISYDSVGSGAGVKNFLAQEGDFGASDAPLRDDERQKFPENRGKPIQIPMTGGLLVFAYNLANYEGLDDIRLSRNTYCGIVTGKITKWNDPAIVADNPDVRLPDIPLLFVHRSDGSGTTFIFTSHIEAVCPDWKAGSGKEVEWPIGIPAPGNEGVSTQIQQTAGAIGYIEYSYSKDKNLQTAVLENKSGNFIKPSPENASKAFVGQTVPDDFALVIPDTESPDAYPIVGLTWLLLYTNYDDPSKAEVVKNFVQWSLVEGDESASTLGYLPIPDDLQEKVITTLNQSL

SEQ ID NO: 13 - protein, 214 aa, Cyanobacterium sp.
MVEGSRLQRHTSEVAQDVLRMGALVEESFRYSHQALFEGDLETVALITNQDIEIDRYYRHIEMRCATILTLQAPVAQDLRILSAFMQLVRDLERIGDYAKDLGQIAIKLALYPSHSCMPELAAMSKHAQVMLAKAMVALSELDSQAGEKIKLLDDTVDNAYDKLYHTLAQQRDIKGVVEPIILLALAIRHIERMADHATNIAQRVSYIVTGERN

SEQ ID NO: 14 - DNA, 129 bp, Cyanobacterium sp.
gagctctagaaaaactattgacaaacccataaaaaatgagataagattatagattgtcactggtattttatactagaggcaaattatatttatatatacaaaaatgctgtataaaaaacatctcatatg

SEQ ID NO: 15 - DNA, 327 bp, Cyanobacterium sp.
tgagaaaaagtgtaaacaaatattaagaaaaagatcagaaaaatttaacaacacgtaataaaaaaatgcgtcactacgggttataaatttacatgaaaggttaaaacacttttctgagacgattttgataaaaaagttgtcaaaaaattaagtttctttacaaatgcttaacaaaaacttggttttaagcacaaaataagagagactaatttgcagaagttttacaaggaaatcttgaagaaaaagatctaagtaaaacgactctgtttaaccaaaatttaacaaatttaacaaaacaaactaaatctattaggagattaactaagc

SEQ ID NO: 16 - DNA, 469 bp, Cyanobacterium sp.
ctcgagcgctcgttccgcaaagcggtacggagttagttaggggctaatgggcattctcccgtacaggaaagagttagaagttattaattatcaacaattctcctttgcctagtgcatcgttacctttttaattaaaacataaggaaaactaataatcgtaataatttaacctcaaagtgtaaagaaatgtgaaattctgacttttataacgttaaagagggaaaaattagcagtttaaaatacctagagaatagtctggggtaagcatagagaattagattagttaagttaatcaaattcagaaaaaataataatcgtaaatagttaatctgggtgtatagaaaatgatccccttcatgataagatttaaactcgaaaagcaaaagccaaaaaactaacttccattaaaagaagttgttacatataacgctataaagaaaatttatatatttggaggataccaaccatg

SEQ ID NO: 17 - DNA, 450 bp, Cyanobacterium sp.
aatgtactcaaaaaaaagttattatcctaaaaaaatattgtttattgtcgattgtaaagacttaattgtcaattatgaaatgctagagaaaaaatttattattcatatttgtttttatctatagtaaaactgctaaattttattatctacatcaactaatcaaaagttaagaaaaaagatagaaacgcccatgaatattaaagattaatctgtgtcctttaactttttatccccttaaaagagcataactaaaacattgatagattttataaagaaaagtaacaaaatcttgacttaaatgagaaaggattaaaaaccaaagccttatctgagggaatgttaaacaaattttaaatattgttaagcaagaaccacaatggtgacaaatagcccttatcatcttcagtaatgtagtagtttaagtatttgtcgagagaggaatccctcatg

SEQ ID NO: 18 - DNA, 321 bp, Cyanobacterium sp.
ttttaattcaaatattatttagtcacgactcttttgtcctctaagctcagaaccagaagtcaataaagggcaaggagcaaagatagataatgatatatagggttggggtgaataattaataacttctaactctgaactcctaactcccgactcttgccattatttctaagaaaagatagaatttttaacataaaattaatactttagggatattctatcaaaaaaccataaaagcgaaaacccaaaaaggttatactaaactagatctatccccaaatttagaaaaaaaaattaaatcaaataggaggagcgtagtcgatg

SEQ ID NO: 19 - DNA, 202 bp, Cyanobacterium sp.
ttaaataacttagatctaggataactaatagcaataaattcaggttttaggttttaggttcaataggctcatcgtgtaacgaaacctagtgcctaaagcctaagcactgaacaaaatacacaattgcagtaatacttctaagcatagttcgagtttagaaaaacggcacaagtaaattctgacttcaagtcaaattatgatg

SEQ ID NO: 20 - DNA, 405 bp, Cyanobacterium sp.
atctgtcgacgagaaggggaacagggaaaagtatttataattgatacaaactgtggttcaacttattttaaagacatttttctccatttaatgattatttcggggaaaattttgaggatttttgattcttaaattgacgatattttgtcactaacacaacgtgagcggtaaatttatatatagacctaaaacctttactataagtgttatatatttaaatcgctaagtatatagttaaagtgtagccaataattaacttttaacaagtgattaccgttaagtcccttaatttatcactacaagctaaaacaaatttttcaattagatatgacattaggtcaaagttcatagtatgatagtaaaaaataaaatttgacgatctgtaaaaataaaaaaacacaatga

SEQ ID NO: 21 - DNA, 568 bp, Cyanobacterium sp.
gtcgacctccttaatccgattcctgcaaatggtctgcaacttcccgatacaaattcatcacatgattatccgccaagctgtagtaaacattacggccgacccggcgatactttaccaggcgctgcgatcgtaaaattcgtaattgatgggaaactgccgattcactcactttcatcgccgctgctaaatcacagacacagagttcttggcgggccaatgccgacattaaacgcaaccgactcggatcagctagtgcactgaaaaactccgccatttgctgggcctggtccaatgacatcacctctggttgaacctgtcgtacctgctcaagatgaacaagaggttgatcacaaaggggcatctcttcgttctggcaggattgtgactttgacaacgaggacttactcatagaggttggcgttaggagctagggaaaaatttaaactggatttagaaaatgattttcatcctaacatctttaatatctgagcatatcttcaggtgtttcaagatttgtgctacggttcaaggaggtttttctttaaatcacgttggccgccatgaattc

SEQ ID NO: 22 - DNA, 649 bp, Cyanobacterium sp.
gtcgactaaatcgtaatacctaaatcagccaacaaaatttagcacaattgcacaggggagaagttcagattaatacatttatactattaatttgcgatcaccctgtgccagttgcgtaagtattgttttccactaaagagcgatataagttaatgacgtgactgtcagccaaactataataaacattccgaccttctcgacgatagctgactaaacgcatagctttcaataaccgcagctgatgacaaacagctgattcactcattttggttaatgcagctagatcgcaaacacacaactcactagaagccaaagctgataggaggcgtatacggtttgtatctgctaacaccccaaaaatttctgccatttgttgtgctttatctgtcggtaagatttgagcctgagatgagcgtacattatctagatgcaccagatgagtatcacaggtaggggtatcagaactttgaattaagtctaagtcctgctttttcttgtgcttattcatagcaagttttacttagcaatagttatcaatctcaataatacctaaaatgataaccattgtacaattgaatagttgttcaattgttgtattagaatattggcagttaactttttgccttaattctaaagctgctatgaattc

SEQ ID NO: 23 - DNA, 503 bp, Cyanobacterium sp.
gtcgacgggcaaactttatgaagcagatcaagcctatatccgccaagcaaccggcagccgcgttgattagtgggtgtgtccatcctctggttcgtctaggtgctccgaagcgtcacgatagagattaagaatgtggtgatccttgaggcgataaatcacattccgcccttccttgcgatagctcactaaacgtgctgtgcgcagggttcttagttggtgagagacagccgattcactcatttcaacggcggcggcgagttcccccacccgcatctctccagtggccagggccgaaagaatacgccagcggttggcatcccccaagacaccaaaaaattcggccatccgttgggccttggcttggttcaagattttgccactgtggtctgtcattgttcgctgatctaaacaatacctgaataattgttcatgtgttaatctaaaaatgtgaacaatcgttcaactatttaagacaataccttggaggtttaaaccatgaattc

SEQ ID NO: 24 - DNA, 1256 bp, Cyanobacterium sp.
gtcgaccatgcgtccaaaactttcaccatcctttccctatcaacctttactgcactaaagacaagtgagatagcagtggcaatctggctttgcaatcaatgtttccactaaagcgtttagcgttactgcggctagaagtcctccaccgaggctcccctgaatggtgatatggggaatgggactggtcatcagtcgtcgttttgcccccggagcatgactaaaaccgatcggcattccgatcacaagagccggctgaatatgttgttgctctatcagcttacaggcagtgagtaaaacagaaggggcatagccgatcgccagcacacatccttggggaatctgttgtaaccgctgttgccaatggtcatggtgccaaaaagcttgctcggcttccctaagccctgtgatgtgagggtcgtcaatcagcgttttaaccgtacatcctaaatgagctaaccgagtttgatcaagagccgcagccacaaccggaacatcggtgacgactggacaccctgctttcagtgcatctcgtgccgaggcgatcgctccctgactcaatcgaacggcgtttaccaagctaacatcaccacaggccagcactaattgatgtagtaagtgaatggtaatttcagagtaagccgataaatccggtagcaggtgtttgagggattcctgaaaggcttctggatgagttgttgtctccgcatctaggttcgtccacaactgatcgagttttcctaacccctcctggacatccacatcaagctgtttcagttgggccagagcttccgcttgggtaatctggcaactctggtcgcgtcccagtaatccttctaaagcagatgcggtttggcggagtcgagtaatctgctgaatcacagcctgatattgctgttgcaactgcaccattagggtgggatcaaggctctcttcagaatggctatccagcagttgccgaatatgagacaactgaaagccctgctgtttgagggcaatgactcgttggagccgttgtacgtcctgctgagtataaaggcggtagttgccctctgagcgttgaacggggggaagcaatcccagggtgtggtaatggcgcaccatgcgaggcgtaacgccacctcccactgcatctgtgagttctttaatcgttaagtgattagtcttcatccctttagtttactcaaaaccttgacattgacactaatgttaaggtttaggctgagaaggtaaaaatccaagttaaaaagcatgaattc

SEQ ID NO: 25 - DNA, 2280 bp, Cyanobacterium sp.
gtcgaccctatatcgggcttttctcaataaaatctttattttttgaggtgctttttagccataaataatcactttagtataaaattttgacggcgtaaagttgataaaatagaattaagaatggactatcggtacagaaaaaatgggtaactggatggtgaataaacttcccttacccaatgcactctccaccgttaaagaccccctatgcttaacggtgatcacctgggcaatggcgagtcccaaccctgtcccccccgttttgcgcgaacgatctcgattaactcggtaaaaacgctcaaaaatgtgttcctgttggtcgggggcaatgccgatgccggtatcttgcacggtgatgatagccatctgttcatgggatgtcagggtaatatcaacacgtcccccagcagttgtgtattgaatggcgttggcaattaggtttgagaccagtcgatagagttgggattcattaccccaggcgtaaacttcccctgaactcagatcactgctgagatcaatgtgggcggcgatcgctaattctaaaaactcttcggtgaggtcactgactaaatcatttaaacaacaaagccgccaatcttcggcggtggtttcctgctctaagcgacttagtagcaataaatccgtaatcaattggcttaatcgccttccctgtcgttcaacggtatgtagcatggtgttaatttctggggaatggcttgagtcgatgcgtaataccgcttccaccgtggccaacagactagccaatggcgatcgtaattcatgggctgcattcgcggtgaattgttgttgttgttggtaggactggtaaatgggacgcatggctaaccccgctaagccccaactggagaaggcgaccaaacccagggcaatgggaaaactaagccctaaaatccaaagaatacgtttattttcggcatcaaaggctgccaggctccggccaatttgtagatagccccaggaagatttgtctgtattaccggcgctatgcaaaatggtggtgaattgtcgataccgatcgccggttggggggtgaatagtctgccaagtttcctggttaaaaatggaggatagggaagccggttgattaggcgaaaaagccagcaggttgccttgataatcaaataaacgaatgtaatataaactgcgatcactaatgcccaacgtgtgacgttcaatcagggtggggttgacctggcagggttggttgaccaaacacagatcgggcaacattttttgtaatactccggtgggactagcattactcggcaacatcggctctaaactgtcatgcaacgtcccggcgatcgactccacttctcgctccaacgccatccagttggcctgcacaatggcacgataaacccccaaccccaacagggtaagaataccccccattactagggcataccagaaagccaattgcagacgactacgggcaaagaggcgacgggtattcatggcgatagggtgaaccgatagccttgaccgggaactgttttaattgggcaaggacaattttgttgagctagcttgcgtcgtatcaaacgcatttgggccgccaccacattactcatgggctcctcatcaagatcccacagttgttgccggatcttgctaccggaaatgatccgctctgggttttgcatcagatattgaaaaatttgaaattctcttacggttaaagcaatttcctgtctttctaggtttagtggctccgagatagttaccgataacagattattactgggatcaaggctgaagttgcccaaagttaaaatttgcggttggaattgtggcgatcgccgttgtagtgcccgcagtcttgctaatagctctgccatcacaaacggttttgttagatagtcatctgccccggcatctagtccttcgacacggttttccggttctcctaacgctgttaacatcaacaccggcaaggaattaccctgggttctcagtttttgacagagttccaaacccgataatcccggcagtaaccaatccacaatggcaagggtgtattccgtccattgattttccaaataatcccaagcttgggagccatccgtcacccaatccaccacatacttttcactaactagcactttcttaatagccattcccaaatccgtctcatcttccaccagcaaaattcgcatcgcctctgccttttttataacggtctgatcttagcgggggaaggagattttcacctgaatttcataccccctttggcagactgggaaaatcttggacaaa

SEQ ID NO: 26 - DNA, 330 bp, Cyanobacterium sp.
tggtcaagttactatatgtttagaaacaacaaaaaaagaagtcattataaaaataattgatacaggaattggcattaataaagaagaacaaaaattaatttttaatcgtttttatcgaatcaataaagcaagaaatagagagaaaggcagttgcggattaggtttagctattgcaaatgcgatcgcgcttaatcatggtggtagaataattttagaaagtcaagaaaatcaaggcagtatttttaccgtttatttaccgaaaatcatttcatcctaatttcatattcttttgacagaatcaaaggtaaagataaaaagagagaaacagtc

SEQ ID NO: 27 - DNA, 320 bp, Cyanobacterium sp.
gaatatctcatccttagcttctacttataccttcagcatagttaaaaatcatccctttattgatggtaataaaagaacaggttttattagtggagtaacctttttaatgctcaatggttctcactttactgcttctgaagtggaagtagtacatatcatccaaaccttagctagtggcagaattaccgaggaagaattacaacaatggttcgtaaggaaaagtaagcagatgaataattaaagcatcatttcatcctcatttcatattctcctgtcaccatggtatggaagattaggtaaaaatgaggaaaaagtttatt

SEQ ID NO: 28 - DNA, 309 bp, Cyanobacterium sp.
atacatggttggttcactgacttttaccccagttttctctttgaacaattggcataactctgaaaaaatcagatcgggcttttgttgaattatttgttcaatcaaagcaaaaccgtgattgtctattttcttttttttcccaccactcatagataaaaatttatcccgaactcaggttatattaagttcggatgatcacttaagataattgatcagattggttaagatagagaaaaattctttttcatagtgatttcataattgatagttacaataacgattattatttagtaaaaagattttcaaatc

SEQ ID NO: 29 - DNA, 246 bp, Cyanobacterium sp.
ccaatatcttgtcatacatacttatttgcctcactattagccctatatgtctctattgtatttttctttttctcctattcctagatcttgtaatgaatcattactctctgaaatatagctactaattttatggttgtttgtaaaatatattaacaaatgaacaataaatcatattttgtgttaatctaattattagacaactactgaatttatattcagatattcacagataggagaattttgatt

SEQ ID NO: 30 - DNA, 539 bp, Cyanobacterium sp.
attctgtgaattgattagatttgaggttttttaagaggttgattaccttgcctccaaaaaaatcataacacactaatgctctatatgaaagggctttagacccataggtttttgagaaaaaaacttgctaactctcggacaatgtcagcataactaaagtcaattcttttcgtactttataattgtctataatttaatatacaactgttctgaaactagtttttctctacattccttagttttatctgagtaaggttgcttgtaacttaacttcggttgggcctaaaaatatccgattaggagcaggtgtcagactttaattaattattaattattaattgcttattgccaaccctcggcgacaccactttttcatcagccccagataaagattgatgttttagttttgtttctttttatcccctaattcaactaatacaagtaaaactaaggttgtttatcaaaaatgatggttgatgtttgggtaaattttaagatattatgaaaagaaaatgaataaaaaatgaaaaatctttatg

SEQ ID NO: 31 - DNA, 411 bp, Cyanobacterium sp.
tatttatatataaactcgaataaaattatcaatataaagtcaaactatatctatcctattttaactgctattggtaagtcccttaattagtgttggggtgaatagattttaaaagggcaaacccccctttatcctccctcgagaggggggagggcaaaaggcaaggggcaagggaaaaattaagaattaagaattaaaaactccgaacacctgtaggggcgaatagccattcgcttcccctcatccccccatctccccaacaccctaagcccctactcgttactcatttatttacatcatttatttacatcattaagaaaagtaacaaattttgacaagtagtcttttgacaggaaaaagcaaattctcgaagatgaaaacaatagaaaaaaattcaatcttacagtaacg

SEQ ID NO: 32 - DNA, 281 bp, Cyanobacterium sp.
  misc_feature (1)..(116): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgcaaaaaacgaatnnnnnnnatgtgtaaaaagaaannnnnnnnnnnnnnngtagtcaaagttacnnnnnnnnnnnnnnnnnnnnnntaatgtnnnnnnnnnnnnnnnnnnnnnnnnnnnnnnnnnnnnnnnnnnnnnnnnnnnnnnnccgaggacaaannatg

SEQ ID NO: 33 - DNA, 166 bp, Cyanobacterium sp.
ctattatttttcgtttatatgcagatttagaataaacaaaattcatttactgcaaattttcaaaaaaatgtgactaaacatacaaaataaagaaaaaataaagttttaaatttatgtacatcaaacttaagaaatgtttaaattacttagaaatttatagttcatg

SEQ ID NO: 34 - DNA, 352 bp, Cyanobacterium sp.
tattgttctaaaaaaggtttaatgcttactaaataaataattaatttgcctgtctcaaaaagacaggttttttttatgaaagtaataagaaataagtagaagtgaggagttggaaagataggattaagaattaggagttaactattttcattctttattcttccattgcccattgagaaatcatatctaaaatcagcaacgccaaattttagatgcaaaataaccataaataaaatgcagaaaaaagaatactttagatcttccgtatcagaagatacatttcttaacaaaatctggtgacaagattaaacacacgaaatccgaggttttatatattgattagtcctagatg

SEQ ID NO: 35 - protein, 569 aa, Zymomonas mobilis; identical to SEQ ID NO: 4

SEQ ID NO: 36 - protein, 558 aa, Zymobacter palmae
MNSYTVGMYLAERLAQIGLKHHFAVAGDYNLVLLDQLLLNKDMEQVYCCNELNCGFSAEGYARARGAAAAIVTFSVGAISAMNAIGGAYAENLPVILISGSPNTNDYGTGHILHHTIGTTDYNYQLEMVKHVTCAAESIVSAEEAPAKIDHVIRTALRERKPAYLEIACNVAGAECVRPGPINSLLRELEVDQTSVTAAVDAAVEWLQDRQNVVMLVGSKLRAAAAEKQAVALADRLGCAVTIMAAAKGFFPEDHPNFRGLYWGEVSSEGAQELVENADAILCLAPVFNDYATVGWNSWPKGDNVMVMDTDRVTFAGQSFEGLSLSTFAAALAEKAPSRPATTQGTQAPVLGIEAAEPNAPLTNDEMTRQIQSLITSDTTLTAETGDSWFNASRMPIPGGARVELEMQWGHIGWSVPSAFGNAVGSPERRHIMMVGDGSFQLTAQEVAQMIRYEIPVIIFLINNRGYVIEIAIHDGPYNYIKNWNYAGLIDVFNDEDGHGLGLKASTGAELEGAIKKALDNRRGPTLIECNIAQDDCTETLIAWGKRVAATNSRKPQA

SEQ ID NO: 37 - protein, 339 aa, Lyngbya sp.; identical to SEQ ID NO: 2

SEQ ID NO: 38 - protein, 334 aa, Microcystis aeruginosa
MIRAYAAQEKGGKLEPFDYDPGILADEDVEIAVEYCGICHSDLSMLDNDWGLTTYPFVPGHEVVGTIAALGAKVKELKLGQRVGLGWFSRSCSTCETCMSGDQNLCATAEGTIVGRHGGFAERVRAHHSWLVPLPDQLDAAKAGPLFCGGITVFNPIVQFNIKPTARVGVIGIGGLGHIALKFLKAWGCEVTAFSSSPDKETEAKELGATHFINSRDPEALQSVQNYFDFIISTVNVNLDWGLYIACLRPKGRLHIVGAVLEPMATYAFPLIMGQKSISGSPLGSPSTVSKMIEFASRHGIEPVTETYPISRVNEAMEKLRTGQPKYRLVLQIK

SEQ ID NO: 39 - protein, 335 aa, Synechococcus sp.
MPMIKAFAVHESDGDLQPFEYDPGALLSDQVEIEVKYCGICHSDLSMISNEWGMTQYPLVPGHEVVGAIAKVGENVKNLSVGQIVGLGWHAGYCNECPQCTTGDQNLCATAQGTIVGHHGGFAEKVRAAANSVVPIPEGIDLEAAGPLFCGGITVFNPLVQYGIQPTAKVAVIGIGGLGHMAVQFLNAWGCEVTAFTSSEAKITEALELGAHHTLNSRDPEAIAAAAGQFDLIISTVNVKLDWNAYLSTLKPHGRLHFVGATLDPLDINVFALIMQQRSISGSPVGSPATIAKMLEFAKLHKIQPKIETFKFEDVNQAIARLKSGEAHYRIVLCR

SEQ ID NO: 40 - protein, 336 aa, Synechocystis sp. PCC 6803; identical to SEQ ID NO: 7

SEQ ID NO: 41 - protein, 29 aa, Cyanobacterium sp.
  misc_feature (17)..(17): Xaa can be any naturally occurring amino acid
VNYQSVGSGAGLRQFIXGTVDFAGSDLPL

SEQ ID NO: 42 - protein, 13 aa, Cyanobacterium sp.
  misc_feature (10)..(10): Xaa can be any naturally occurring amino acid
DLERIGDLAXNIA

SEQ ID NO: 43 - protein, 13 aa, Cyanobacterium sp.
  misc_feature (10)..(10): Xaa can be any naturally occurring amino acid
DLERIGDLAXNIA

SEQ ID NO: 44 - protein, 222 aa, Synechococcus sp. PCC 7002
MQLFHQSPQRERTQFERSLQRLEQEVLRMGALVEQAFRLGHNCLFKEDLEAYETLRALDKEIDRYYRQIEADCSAMLTLKAPVSQDCRFVSAFMQLVRDLERIADYAEDLAEKSMRLVPYPTHILMADIEGMSEHAQLMLSTSLVALADLDPNAGAYVKKLDDRVDDDYDRIYAALAQQQNQPGVIEPFILMALIIRDLERMADHATNIAQRVSYIITGQRG

SEQ ID NO: 45 - DNA, 4848 bp, Artificial sequence
  Synthetic suicide plasmid designed for knock-out of PhoU gene in Synechococcus PCC 7002.
tcccttaacccaacaaagcaaccgcttacctctataaattcaaacagtaatttaccacgggttgcagaagcccaaagcccttataaagcaaacttcccagagaaatctgaacagcccaaagacccacccatttgaagcttctcgatagctaaaatatgctttgattctgcctataaagcatccctttttacgaagaaaacccctcgaaaaaaccgttaaccaaactataacttaatcttttttaaactttggaattataatgtaagaatagtctctataaaaaacgatataaagaaacgcaatttaaaatctatgcaattatttcatcagtcgcctcaaagagaacgaacgcaatttgaacgttctttacaacgtttagaacaagaagttttgcggatgggtcgactctagaggatctcaatgaatattggttgacacgggcgtataagacatgttatactgttgaataacaaggacggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccggggatccactagagcggccgccttagcccagcagcaaaatcagcctggggtcatagaaccatttattttaatggcgttgattattcgggatctagaacgcatggctgaccacgcgaccaatattgcccagcgggtttcttatatcatcaccggacaacggggctaacacccctctagctccctttttgagaaacatccacacccctctagctccccttgaggggagaagattggccaccttccccaaggcagaaccccttccggcctagcggccaccttccccaaggaaggatagtgggtttattgtgtggcgtcggagaggtcgaccacgtcaagccagccttcgagggcatcccagccgaggcctttacggatgagggtcggttggcgatcgcaaaagtcgagaatcgttgaagttttggtgccactagtgatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgca

SEQ ID NO: 46 - protein, 222 aa, Anabaena variabilis ATCC 29413
MKAVVYSPNPENPQLARAIRRLERDVLRMGALVEQSFRLSHQALFSRDLKAAEELPRLDKKIDRFYRQIESDCTSIMTLQAPTAQDLRCLSAFMQLVRDLERIGDYAKDLAQIAVKIFPYPTHPSLPEIAVMAHHAQAMLATSLVALADLDEAGGRSIKHLDDAVDNAYDHLYQTLSQQRDVQGVVEPIILLALAIRCLERMADHATNIGQRVAYIVTGQRA

SEQ ID NO: 47 - DNA, 8105 bp, Artificial Sequence
  Synthetic suicide plasmid designed for knock-out of phoU gene in Anabaena variabilis ATCC29413
tcgctcatatgacgaatcacgcaacaagagagtgaaaatttcttttctctctacctaaataatctctaattcctacgttgaggtcagagtctggtgatgataatcaaccaaagctttacttaagactctttggttaaatgtaatactttttactatgtgttttagctgtcgttataagctaatctagttttgtaagtttaagaaaagataaacgattggtaaaatatcacctttaataaaaaaaatcccgctcctgggagggatgggattcgctaaaaaatactccagctacaattaggcacgttgaccagttacgatatatgccactcgttgaccgatgttagttgcatgatcagccatacgttctaaacaacgaattgccagtgctagcagtatgattggctcaacaacgcgtcgactctagaggatctcaatgaatattggttgacacgggcgtataagacatgttatactgttgaataacaaggacggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccggggatccactagagcggccgcttccggtagagatgggtgagtcggataaggaaagattttgactgctatttgagctaaatctttggcataatcaccaatacgctctaaatctctgactaattgcataaaagcactcaaacagcgcaaatcttgggctgtaggtgcttgtaatgtcattatggatgtacaatctgattctatttgtctataaaaacggtctatttttttatctaatcggggaagttcctcagccgcttttaagtcgcgggagaataaggcttggtggctgaggcggaatgattgttctaccaaagctcccatgcgtaatacatcccgttctaaacgcctaatggctcgtgctaactggggattttcagggttgggactataaacgacagctttcacacttgtagtctcaaacgtgaagatatctttaactatagtcttggcgttgtcagtttgccactacttcaggtatgtgaagttgtatccatgcgccaccgatttctgaatggttcatggctttgatagagccaccgtgagctaaaaactagtatagtctatagtccgtggaattattatatttatctccgacgatattctcatcagtgaaatccagctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgagatctagatatcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgatgaattcgcgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcaggtcgactctagctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggaggtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatatcctattggcattttcttttatttcttatcaacataaaggtgaatcccatatgaactatataaaagcaggcaaatggctaaccgtattcctaaccttttggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagcctgtcatacgcgtaaaacagccagcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgca